SEQUENCE LISTING (24 sequences)

SEQ ID NO: 1 - protein, 15 aa, Homo sapiens
  REGION 1..15: note = Description of sequence: HER3 ECD P12 (aa 57-71)
EVVMGNLEIVLTGHN

SEQ ID NO: 2 - protein, 15 aa, Homo sapiens
  REGION 1..15: note = Description of sequence: HER3 ECD P81 (aa 401-415)
SWPPHMHNFSVFSNL

SEQ ID NO: 3 - protein, 15 aa, Homo sapiens
  REGION 1..15: note = Description of sequence: HER3 ECD P84 (aa 416-430)
TTIGGRSLYNRGFSL

SEQ ID NO: 4 - protein, 15 aa, Homo sapiens
  REGION 1..15: note = Description of sequence: HER3 ECD P91 (aa 450-464)
SAGRIYISANRQLCY

SEQ ID NO: 5 - protein, 15 aa, Homo sapiens
  REGION 1..15: note = Description of sequence: HER3 ICD P38 (aa 850-864)
VADFGVADLLPPDDK

SEQ ID NO: 6 - protein, 15 aa, Homo sapiens
  REGION 1..15: note = Description of sequence: HER3 ICD P41 (aa 865-879)
QLLYSEAKTPIKWMA

SEQ ID NO: 7 - protein, 15 aa, Homo sapiens
  REGION 1..15: note = Description of sequence: HER3 ICD P52 (aa 920-934)
VPDLLEKGERLAQPQ

SEQ ID NO: 8 - protein, 15 aa, Homo sapiens
  REGION 1..15: note = Description of sequence: HER ECD P12 (aa 56-70)
CEVVMGNLEIVLTGH

SEQ ID NO: 9 - protein, 15 aa, Homo sapiens
  REGION 1..15: note = Description of sequence: HER3 ECD P91 (aa 451-465)
AGRIYISANRQLCYH

SEQ ID NO: 10 - protein, 15 aa, Homo sapiens
  REGION 1..15: note = Description of sequence: HER3 ICD P86 (aa 1090-1114)
GCLASESSEGHVTGS

SEQ ID NO: 11 - protein, 15 aa, Homo sapiens
  REGION 1..15: note = Description of sequence: HER3 ICD P89 (aa 1115-1129)
EAELQEKVSMCRSRS

SEQ ID NO: 12 - protein, 15 aa, Homo sapiens
  REGION 1..15: note = Description of sequence: HER2 MHC Class II P42-56
HLDMLRHLYQGCQVV

SEQ ID NO: 13 - protein, 17 aa, Homo sapiens
  REGION 1..17: note = Description of sequence: HER2 MHC Class II P98-114
RLRIVRGTQLFEDNYAL

SEQ ID NO: 14 - protein, 18 aa, Homo sapiens
  REGION 1..18: note = Description of sequence: HER2 MHC Class II P328-345
TQRCEKCSKPCARVCYGL

SEQ ID NO: 15 - protein, 15 aa, Homo sapiens
  REGION 1..15: note = Description of sequence: HER2 MHC Class II P776-790
GVGSPYVSRLLGICL

SEQ ID NO: 16 - protein, 15 aa, Homo sapiens
  REGION 1..15: note = Description of sequence: HER2 MHC Class II P927-941
PAREIPDLLEKGERL

SEQ ID NO: 17 - protein, 15 aa, Homo sapiens
  REGION 1..15: note = Description of sequence: HER2 MHC Class II P1166-1180
TLERPKTLSPGKNGV

SEQ ID NO: 18 - protein, 9 aa, Homo sapiens
  REGION 1..9: note = Description of sequence: HLA A2 restricted HER2 peptide (cross reacts with HER3) P369-377
KIFGSLAFL

SEQ ID NO: 19 - protein, 9 aa, Homo sapiens
  REGION 1..9: note = Description of sequence: HLA A2 restricted HER2 peptide (cross reacts with HER3) P698-697
RLLQETELV

SEQ ID NO: 20 - protein, 21 aa, Homo sapiens
  REGION 1..21: note = Description of sequence: MHC class II recognizing multi-epitope peptides p5
ELAAWCRWGFLLALLPPGIAG

SEQ ID NO: 21 - protein, 21 aa, Homo sapiens
  REGION 1..21: note = Description of sequence: MHC class II recognizing multi-epitope peptides p435
IRGRILHDGAYSLTLQGLGIH

SEQ ID NO: 22 - protein, 21 aa, Homo sapiens
  REGION 1..21: note = Description of sequence: MHC class II recognizing multi-epitope peptides p1209
SPPHPSPAFSPAFDNLYYWDQ

SEQ ID NO: 23 - protein, 15 aa, Homo sapiens
  REGION 1..15: note = Description of sequence: HER3 ECD P12
CEVVMGNLEIVLTGH

SEQ ID NO: 24 - protein, 15 aa, Homo sapiens
  REGION 1..15: note = Description of sequence: HER3 ECD P91
AGRIYISANRQLCYH

(19) United States
(12) Patent Application Publication
(10) Pub. No.: US 2026/0232728 A1
(43) Pub. Date: Aug. 13, 2026

(54) METHODS FOR IDENTIFYING DISSEMINATED CANCER CELLS IN BREAST CANCER PATIENTS

(71) Applicant: H. LEE MOFFITT CANCER CENTER AND RESEARCH INSTITUTE, INC., Tampa, FL (US)

(72) Inventors: Brian CZERNIECKI, Tampa, FL (US); Ganesan RAMAMOORTHI, Tampa, FL (US)

(21) Appl. No.: 19/150,723

(22) PCT Filed: Jan. 26, 2024
(86) PCT No.: PCT/US2024/013114

§ 371 Date: Jul. 24, 2025

Related U.S. Application Data
(60) Provisional application No. 63/441,302, filed on Jan. 26, 2023.

(51) Int. Cl. A61K 35/15; A61K 9/00; A61K 35/17; A61K 40/11; A61K 40/19; A61P 35/04; C07K 14/57; C12N 5/00; C12N 5/0784; G01N 33/57515

(52) U.S. Cl. CPC A61K 35/15; A61K 9/0019; A61K 35/17; A61K 40/11; A61K 40/19; A61P 35/04; C07K 14/57; C12N 5/0018; C12N 5/0639; G01N 33/57515; A61K 2239/31; C12N 2501/11; C12N 2501/392; C12N 2501/60; C12N 2501/727

(57) ABSTRACT

Disclosed are methods for the detection and isolation of disseminated cancer cells and the used of tumor antigen pulsed type 1 dendritic cell vaccine for the treatment and prevention of metastasis and abscopal tumors.

### II. CROSS REFERENCE TO RELATED APPLICATIONS

This PCT application claims priority to, and the benefit of, U.S. Provisional Patent Application No. 63/441,302, filed Jan. 26, 2023, which is incorporated by reference herein in its entirety.

### I. STATEMENT REGARDING FEDERALLY SPONSORED RESEARCH

This invention was made with government support under Grant Nos. W81XWH-16-1-0385 and W81XWH-19-1-0675 awarded by the Department of Defense. The government has certain rights in the invention.

### III. REFERENCE TO SEQUENCE LISTING

The sequence listing submitted on Jan. 26, 2024, as an .XML file entitled “10110-438W01_ST26.xml” created on Jan. 26, 2024, and having a file size of 31,783 bytes is hereby incorporated by reference pursuant to 37 C.F.R. § 1.52(e)(5).

### IV. BACKGROUND

Metastatic spread in breast cancer patients is the major driver of cancer-related death. It was widely thought that dissemination of cancer cells from clinically latent stage of invasive breast tumors manifest metastasis. However, large cohort breast cancer patient studies have revealed that pre-invasive or primary tumors also release disseminated cancer cells (DCCs) as an early hidden event and these DCCs can act as an intermediatory in metastasis outgrowth process. The presence of DCCs in bone marrow (BM) of patients with early-stage disease are considered for a clinical predictor of recurrence in breast cancer. Studies from spontaneous mouse tumor models of breast cancer have demonstrated that early DCCs can reach distant organs and then manifest metastasis. DCCs are known to evade immune responses and remain dormant for prolonged periods of time before developing clinically apparent metastasis in patients. The biology of DCCs seems to be highly divergent and expresses unique gene signatures compared to primary tumors or metastatic disease. Recent studies have also revealed dissemination mechanisms for cancer cells in HER2 spontaneous mouse mammary tumor models and triple negative breast cancer models. Patients with pre-invasive and primary tumors also corroborated these dissemination mechanisms. The reason for the limited effectiveness of state-of-the-art therapy is mainly because of their inability to target DCCs in breast cancer patients. Notably, the heterogenic characteristics of DCCs can survive targeted therapies and may cause cancer recurrence and incurable metastasis. What are needed are methods to detect and target DCCs and thereby reduce or prevent metastasis.

### V. SUMMARY

Disclosed are methods the reducing to disseminated cancer cells.

In one aspect, disclosed herein are methods of treating, reducing, decreasing, and/or inhibiting cancer cell dissemination and/or metastasis in a subject with a cancer (such as, for example, breast cancer, including, but not limited to triple negative breast cancer) comprising administering to the subject (systemically or intratumorally) tumor antigen (such as, for example an oncodriver including, but not limited to human epidermal growth factor receptor (HER) 1(HER1), HER2, HER3, epidermal growth factor receptor (EGFR), c-Mesenchymal to Epithelial Transition (c-MET), B-Rapidly Accelerated Fibrosarcoma (BRAF), KIT, Androgen Receptor (AR), Estrogren Receptor (ER), Kirsten rat sarcoma (KRAS), TP53, or APC)-pulsed Type 1 conventional dendritic cells (cDC1). In some aspects, the oncodriver is a class II peptide which does not require HLA match. In some aspects, the methods can further treat, reduce, decrease, and/or inhibit cancers at sites distant from the tumor microenvironment of the cancer being targeted for treatment (abscopal effect).

Also disclosed herein are methods of treating, reducing, inhibiting, and/or decreasing cancer cell dissemination and/or metastasis in a subject with a cancer (such as, for example, breast cancer, including, but not limited to triple negative breast cancer) comprising obtaining TH1 CD4 T cells; contacting the CD4 T cells with tumor antigen (such as, for example an oncodriver including, but not limited to human epidermal growth factor receptor (HER) 1(HER 1), HER2, HER3, epidermal growth factor receptor (EGFR), c-Mesenchymal to Epithelial Transition (c-MET), B-Rapidly Accelerated Fibrosarcoma (BRAF), KIT, Androgen Receptor (AR), Estrogen Receptor (ER), Kirsten rat sarcoma (KRAS), TP53, or APC)-pulsed Type 1 conventional dendritic cells thereby creating DC1 primed CD4 T cells; and administering (systemically or intratumorally) to the subject the DC1 primed CD4 T cells. In some aspects, the methods can further treat, reduce, decrease, and/or inhibit cancers at sites distant from the tumor microenvironment of the cancer being targeted for treatment (abscopal effect).

In one aspect disclosed herein are methods of treating, reducing, decreasing, and/or inhibiting cancer cell dissemination and/or metastasis of any preceding aspect, further comprising detecting the presence of disseminated cancer cells (DCC) in the subject, the method comprising obtaining a tissue sample (such as, for example, blood, bone marrow, or tissue biopsy) from the subject, performing proteomic assays (Flow Cytometry, Immunohistochemistry, Prelude DCIS identification assay). In some aspects, the method of detection comprises imaging (Positron Emission Tomography (PET), Computed Tomography (CT), Magnetic Resonance Imaging (MRI), X-ray)

Also disclosed herein are methods of treating the subject with a detected disseminated cancer cell (DCC). In some aspects the method of treatment comprises hormone therapy, immunotherapy, targeted therapy, chemotherapy, or combinations thereof.

### VI. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, 1G, 1H, 1I, 1J, 1K, 1L, and 1M show the gene expression status of DCCs, primary tumors and metastatic tumors. FIG. 1A shows Immunofluorescence (1F) staining for HER2⁺CYT8/18⁺Ki-67⁺ DCCs detection in the BM. Scale bars, 100 μm. FIG. 1B shows Schematic showing the methods for DCCs isolation from the BM of BALB-neuT mice. FIG. 1C shows the percentage of EpCAM+ DCCs isolated from the BM of BALB-neuT mice. FIGS. 1D, 1E, and 1F show heat maps showing differentially expressed cancer stemness, EMT and cell cycle genes in DCCs of BALB-neuT mice, EL (early lesion tumor cells from BALB-neuT mice), and metastatic TUBO cells. Also see FIG. 9. FIG. 1G shows a schematic depicting the methods for DCCs isolation from the BM aspirates of HER2+ BC patients. FIG. 1H shows IF staining for HER2⁺CYT8/18⁺ DCCs detection in the BM of HER2+ BC patients. Scale bars, 100 μm. FIG. 1I shows the percentage of Pan-CYT⁺ DCCs isolated from the BM of a HER2+ BC patient. FIG. 1J shows bright field image of in vitro cultured DCCs from (I). Scale bars, 10×. FIGS. 1K, 1L, and 1M show heat maps with differential expressions of cancer stemness, EMT and cell cycle genes in DCCs of HER2+ BC patients, JIMT-1 and BT474 cells. Also see FIGS. 10-13.

FIGS. 2A, 2B, 2C, 2D, 2E, 2F, 2G, 2H, 2I, 2J, 2K, 2L, and 2M show intratumoral activation of anti-tumor CD4Th1 cells inhibits spontaneous metastasis. FIG. 2A shows MRI images of BALB-neuT mice treated with or without intramammary gland HER2-DC1 (n=6/group). Also see FIGS. 14A and 14B. FIG. 2B shows the total tumor burden MRI from (A) (n=6/group). FIGS. 2C, 2D, and 2E show immunohistochemistry (IHC) of HER2 and CYT 8/18 double staining for the detection of micrometastases in lung (2C), liver (2D) and brain (2E) sections from BALB-neuT mice treated with or without HER2-DC1 (n=3/group). FIG. 2F shows infiltration of CD4 T and CD8 T cells in mammary gland negative (left) and positive (right) for tumors (n=3/group). Also see FIGS. 14C-14H. FIG. 2G shows IFN-γ secretion after co-culturing TDLNs (from experimental BALB-neuT mice) with or without HER2-DC1 individually pulsed with p5, p435 and p1209 for 72 h (n=3/group). FIG. 2H shows IFN-γ secretion after re-stimulating splenocytes of experimental mice with rat HER2/neu peptides p5, p435 and p1209 individually (n=4-6/group). FIG. 2I shows tumor growth curves of control and intratumoral HER3-DC1 treated BALB/c mice bearing 4T1 TNBC tumors (n=9-10/group). FIGS. 2J and 2K show IHC HER3 and Pan-CYT double staining for the detection and quantification of micrometastases in lung (2J) and liver (2K) sections from (2I) (n=3/group). FIG. 2L shows tumor growth curves of control and intratumoral HER3-DC1 treated C57BL/6 mice bearing B16F10 melanoma (n=10/group). FIG. 2M shows H&E staining for the detection and quantification of spontaneous metastatic nodules in lungs from (2L) (n=7-8/group). All data are presented as mean±SEM. *, P<0.05; **, P<0.01; ***, P<0.001 by one-tailed or two-tailed Student's t test.

FIGS. 3A, 3B, 3C, 3D, 3E, 3F, 3G, 3H, 3I, 3J, 3K, 3L, 3M, 3N, 3O, and 3P show anti-tumor CD4Th1 cells are primarily responsible for metastatic prevention. FIG. 3A shows MRI images of control and HER2-DC1 treated BALB-neuT mice with or without CD4 or CD8 T cells depletion (n=4-6/group). FIG. 3B shows total tumor burden calculated by MRI from (A) (n=4-6/group). FIGS. 3C, 3D, and 3E show IHC HER2 and cytokeratin 8/18 double staining for micrometastasis detection in lung (3C), liver (3D) and brain (3E) sections from (3A) (n=3/group). FIG. 3F shows tumor growth curves of control and intratumoral HER3-DC1 treated mice bearing 4T1 tumors depleted with or without CD4 or CD8 T cells (n=9-10/group). FIGS. 3G and 3H show H&E staining for the detection and quantification of metastatic nodules in lung (3G) and liver (3H) from (3F) (n=3-8/group). Also see FIGS. 15A and 15B. FIG. 3I shows 4T1 tumor bearing mice treated with HER3-DC1 (s.c.) and intratumoral HER3-DC1, immature HER3-iDC, unpulsed DC1 or HER2-DC1 (n=10/group). s.c., subcutaneous. FIG. 3J shows quantification of metastatic nodules in lungs from (J) (n=5-10/group). FIGS. 3K, and 3L show the detection (3K) and frequency (3L) of CD4⁺IFNγ⁺ T cells in control and intratumoral HER3-DC1 treated 4T1 tumors (n=3/group). FIG. 3M shows the 4T1 tumor bearing mice treated with intratumoral HER3-DC1 with or without IFNγ neutralizing antibody (n=9-10/group). FIG. 3N shows quantification of metastatic nodules in lungs from (M) (n=7-10/group). FIG. 3O shows Tumor growth curves of IFNγ knockout mice bearing B16F10 melanoma treated with or without intratumoral HER3-DC1 (n=10/group). FIG. 3P shows the frequency of spontaneous metastatic nodules in lungs from (O) (n=4-5/group). Also see FIG. 15C. Mean±SEM represented. *, P<0.05; **, P<0.01; ***, P<0.001; ns, nonsignificant by one-tailed or two-tailed Student's t test.

FIGS. 4A, 4B, 4C, 4D, 4E, 4F, 4G, and 4H show intratumoral activation CD4Th1 immunity reduces dissemination of cancer cells. FIG. 4A shows IHC staining for HER2, PR, Wnt4 and RANKL proteins in the mammary glands positive for tumors from experimental BALB-neuT mice (n=3-4/group). Also see FIGS. 16A-16C. FIG. 4B shows immunoblots for HER2, PR, Wnt4 and RANKL in the mammary glands positive for tumors from control and HER2-DC1 treated BALB-neuT mice. Also see FIG. 16D. FIG. 4C shows transwell invasion assay for cancer cell migration from early lesion of control BALB-neuT mice or TUBO cells after various treatments. FIG. 4D shows quantification from (C) (n=3-5). Data are mean±SEM. Control+PG vs other groups: **, P<0.01; ***, P<0.001; ns, nonsignificant by one-tailed or two-tailed Student's t test. FIG. 4E shows immunoblots for HER2, p-HER2, PR, Wnt4 and RANKL. Also see FIG. 16E. FIG. 4F shows multiplex IF (mIF) images of HER2, PCK, PR, RANKL, Wnt4 and DAPI in baseline and post intratumoral HER2-DC1 treated tumors of HER2+ BC patients (n=5). Scale bars, 200 μm. Also see FIG. 17. FIGS. 4g and 4H show the frequency of disseminating cancer cell phenotypes in baseline and post intratumoral HER2-DC1 treated tumors of HER2+ BC patients (n=5). Also see FIGS. 16F, 16G and 17.

FIGS. 5A, 5B, 5C, 5D, 5E, 5F, 5G, 5H, 5I, 5J, 5K, 5L, 5M, 5N, 5O, and 5P show anti-tumor CD4Th1 cells target DCCs. FIGS. 5 and 5B show infiltration of CD4 T (A) and CD8 T cells (5B) in the BM of experimental BALB-neuT mice (n=3-5). FIGS. 5C and 5D show HER2⁺CYT8/18⁺Ki-67⁺ DCCs in BM (5C) and lung (5D) of experimental BALB-neuT mice (n=3-6). s.c., subcutaneous. FIG. 5E shows SA-R-gal staining for the detection (left) and percentage of senescent DCCs in the BM of experimental BALB-neuT mice (n=5-8). Scale bars, 4×. FIG. 5F shows immunoblots for HER2, NR2F1, Wnt4 and Twist proteins in BM DCCs of control and HER2-DC1 treated BALB-neuT mice. FIG. 5G shows IF staining of HER2⁺CYT8/18⁺Ki-67⁺ DCCs in BM of experimental BALB-neuT mice. Scale bars, 100 μm. FIG. 5H shows senescence inducing potential of tumor antigen specific CD4Th1 cells secreting IFN-γ on DCCs from the BM of control BALB-neuT mice (left) and percentage of senescent DCCs (right) (n=5-10). Scale bars, 4×. Also see FIG. 19A. FIG. 5I shows the frequency of CD45-EpCAM+ DCCs in the BM of experimental mice bearing 4T1 TNBC tumors (n=3/group). s.c., subcutaneous. FIGS. 5J and 5K show the detection (5J) and frequency (5K) of CD4⁺IFNγ⁺ T cells in the BM of control and intratumoral HER3-DC1 treated mice bearing 4T1 tumors (n=3/group). FIG. 5L shows tumor growth curves of NSG mice (n=3/group). FIG. 5M shows IHC HER2 and CYT8/18 double staining for the detection of micrometastasis in lung sections from (L) (n=3). FIG. 5N shows tumor growth curves of NSG mice (n=4/group). Also see FIGS. 19D and 19E. FIGS. 5O and 5P show IHC HER2 and CYT8/18 double staining for the detection of micrometastasis in lung (5O) and liver (5P) sections from (5N) (n=4). All data are presented as mean±SEM represented. Control vs other groups: *, P<0.05; **, P<0.01; ***, P<0.001; ****, P<0.0001; ns, nonsignificant by one-tailed or two-tailed Student's t test.

FIGS. 6A, 6B, 6C, 6D, 6E, 6F, 6G, 6H, 6I, 6J, 6K, 6L, 6M, and 6N show anti-tumor CD4Th1 immunity induces immune recognition of DCCs. FIGS. 6A and 6B show heat maps with differential expressions of chemokine and chemokine receptors gene signatures in DCCs of BALB-neuT mice (6A) and HER2+ BC patients (6B) treated with or without IFN-γ. Also see FIG. 21B. FIGS. 6C and 6D show differential expressions of antigen presentation genes in DCCs from BALB-neuT mice (6C) and HER2+ BC patients (6D). Also see FIG. 21. FIG. 6E shows MHC I and MHC II expressions in DCCs of BALB-neuT mice (n=3). FIG. 6F shows CD1d+ population in DCCs of BALB-neuT mice, early lesion tumor cells of BALB-neuT mice and metastatic TUBO cells (n=3-5). Mean±SEM represented. Means were statistically cared by one way ANOVA with Tukey's multiple comparisons test. FIG. 6G shows CD1d+ population in DCCs of BALB-neuT mice treated with or without IFN-γ. FIG. 6H shows the percentage of CD1d+ DCCs from (6G) (n=5-6). FIG. 6I shows the mean fluorescence intensity (MFI) of CD1d from (6G and 6H) (n=3-6). FIG. 6J shows NKT and NK cells infiltration in the BM of experimental BALB-neuT mice (n=3). FIGS. 6K and 6L show the frequency of NKT (K) and NK cells (L) from (J) (n=3). FIG. 6M shows the B cell infiltration in the BM of experimental BALB-neuT mice (n=3). FIG. 6N shows the frequency of B cells from (M) (n=3). Mean±SEM represented. Means were statistically compared by two-tailed Student's t test.

FIGS. 7A, 7B, 7C, 7D, 7E, 7F, 7G, 7H, 7I, 7J, 7K, 7L, 7M, and 7N show that CD4Th1 cytokine IFN-γ inhibits tumorigenic and metastatic potential of DCCs. FIG. 7A shows ALDH+ cancer stem cell population in DCCs of BALB-neuT mice treated with or without IFN-γ. FIG. 7B shows CD24+CD44+ DCC stemness population in DCCs of BALB-neuT mice. FIG. 7C shows representative bright field images of mammospheres formed in DCCs of BALB-neuT mice and TUBO cells. Scale bars, 4×. FIG. 7D shows quantification of mammospheres from (7C) (n=3). FIG. 7E shows a heat map with differential expression of cell adhesion genes in DCCs of BALB-neuT mice. FIG. 7F shows SA-β-gal staining for the detection and frequency of senescent DCCs in BALB-neuT mice derived DCCs (n=3-4). Scale bars, 4×. FIG. 7G shows Annexin V⁺7-AAD⁺ apoptotic DCCs of BALB-neuT mice. Also see FIG. 22E. FIG. 7H shows mammospheres formed and their quantification from DCCs of HER2+ BC patients and JIMT-1 cells (n=3). Scale bars, 4×. FIG. 7I shows immunoblots for HER2, p-HER2, NR2F1 and Wnt4 proteins in DCCs of HER2+ BC patients. FIGS. 7J and 7K show heat maps showing differential expressions of genes related to progesterone signaling (7J) and cell adhesion in DCCs of HER2+ BC patients. FIG. 7L shows SA-β-gal staining for the detection and percentage of senescent DCCs in HER2+ BC patients derived DCCs (n=12-15). Scale bars, 4×. FIG. 7M shows the detection and frequency of apoptosis after IFN-γ treatment in DCCs of HER2+ BC patients (n=3). Also see FIG. 22H. FIG. 7N shows tumor growth curves of NSG mice (n=2-4/group). Mean±SEM represented. **, P<0.01; ***, P<0.001 by one-tailed or two-tailed Student's t test.

FIGS. 8A, 8B, 8C, 8D, 8E, 8F, 8G, 8H, 8I, 8J, and 8K show CD4Th1 cytokine IFN-γ mediates gene expression profile changes in DCCs. FIG. 8A shows a volcano plot representation of genes that are differentially expressed in IFN-γ treated DCCs from BALB-neuT mice. FIGS. 8B, 8C, and 8D show the differential expressions of cancer stemness (8B), EMT (8C) and cell cycle genes (8D) in IFN-γ treated DCCs from BALB-neuT mice. FIG. 8E shows a volcano plot depicting genes differentially enriched in IFN-γ treated DCCs of HER2+BC patients. FIGS. 8F and 8G show REACTOME analysis showing differential expression of cholesterol biosynthesis genes in IFN-γ treated DCCs of HER2+ breast cancer patients. FIGS. 8H, 8I, and 9J show cancer stemness (8H), EMT (8I) and cell cycle genes (8J) differential expression in IFN-γ treated DCCs from HER2+ BC patients. FIG. 8K shows the expression status of IFN-stem cell-down signature (ISDS) genes associated with good prognosis after IFN-γ treatment in DCCs of HER2+ BC patients.

FIG. 9 shows gene expression profiles of DCCs from BALB-neuT mice, Related to FIG. 1. FIG. 9 shows heat maps with differential expressions of full list of cancer stemness, EMT and cell cycle genes in BM DCCs of BALB-neuT mice, EL (early lesion tumor cells from BALB-neuT mice), and metastatic TUBO cells.

FIGS. 10A, 10B, and 10C show the expression profiles of selected genes in DCCs compared to HCC1954 cells, Related to FIG. 1. FIGS. 10A and 10B show GSEA analysis with differential expression of cancer stemness (10A) and EMT (10B) signatures in DCCs of HER2+ BC patients compared to JIMT-1, BT474 and HCC1954 cells. FIG. 10C shows heat maps with differential expressions of selected cancer stemness, EMT and cell cycle genes in BM DCCs of HER2+ breast cancer patients and HCC1954 cells.

FIG. 11 shows gene expression profiles of DCCs from HER2+ breast cancer patients compared to JIMT-1 cells, Related to FIG. 1. FIG. 11 shows heat maps with differential expressions of full list of cancer stemness, EMT and cell cycle genes in BM DCCs of HER2+ breast cancer patients and JIMT-1 cells.

FIG. 12 shows the gene expression profiles of DCCs compared to BT474 cells, Related to FIG. 1. FIG. 12 shows heat maps with differential expressions of full list of cancer sternness, EMT and cell cycle genes in BM DCCs of HER2+ breast cancer patients and BT474 cells.

FIG. 13 shows the full list of gene expressions in DCCs compared with HCC1954 cells, related to FIG. 1. FIG. 13 shows heat maps showing differential expressions of full list of cancer sternness, EMT and cell cycle genes in BM DCCs of HER2+ BC patients compared to HCC1954 cells.

FIGS. 14A, 14B, 14C, 14D, 14E, 14F, 14G, and 14H show intratumoral activation of CD4Th1 cells delay primary tumor growth and enhance infiltration of various immune effector cells, Related to FIG. 2. FIGS. 14A and 14B show MRI images of control (14A) and HER2-DC1 treated BALB-neuT mice (14B) were taken at different time intervals (8, 11, 13, 16, 20 and 24 weeks of age). (14C) IHC staining for the detection (left) and frequency (right) of CD4 T cells infiltration in the mammary gland positive for tumors in control and HER2-DC1 treated BALB-neuT mice (n=3). FIGS. 14D and 14E show infiltration of CD4+CD44+CD62L-effector memory (14D), CD4⁺CD44⁺CD62L⁺ central memory (14E) and CD4⁺CD44⁻CD62L⁻ effector cells (14E) into the mammary gland negative for tumors in control and HER2-DC1 treated BALB-neuT mice was analyzed by flow cytometry (n=3). FIG. 14F shows infiltrating level of CD8⁺CD44⁺CD62L⁻ effector memory, CD8⁺CD44⁺CD62L⁺ central memory and CD8⁺CD44⁻CD62L⁻ effector cells in mammary glands negative for tumors in control and HER2-DC1 treated BALB-neuT mice examined by flow cytometry (n=3). FIGS. 14G and 14H show the level of CD19+ B cells (14G), CD3+DX5+(CD49b+) NKT cells (14G) and DX-5+(CD49b+) NK cells (14H) infiltration in mammary glands negative for tumors of experimental BALB-neuT mice (n=3).

FIGS. 15A, 15B, and 15C show Anti-tumor CD4Th1 cells inhibit spontaneous metastasis. Related to FIG. 3. FIGS. 15A and 15B show H&E staining for the detection of spontaneous metastatic nodules in lung (15A) and liver (15B) of experimental mice bearing 4T1 tumors (n=3-8/group). FIG. 15C shows H&E staining for the detection of spontaneous metastatic nodules in lung of experimental IFN-γ knockout mice bearing B16F10 melanoma (n=4-5/group).

FIGS. 16A, 16B, 16C, 16D, 16E, 16F, and 16G show the anti-tumor CD4Th1 response, but not the CD8 T cell response, limits cancer cell dissemination mechanisms, Related to FIG. 4. FIG. 16A shows the percentage of tumor cells expressing HER2, PR, Wnt4 and RANKL proteins (n=3-4). FIG. 16B shows mammary glands positive for tumors collected from HER2-DC1-treated BALB-neuT mice depleted of CD8 T cells and stained for HER2, PR, Wnt4 and RANKL proteins by IHC. FIG. 16C shows IHC staining of NR2F1 protein on mammary glands positive for tumors collected from untreated control BALB-neuT mice, HER2-DC1 treated BALB-neuT mice with or without CD4 or CD8 T cell depletion. FIG. 16D shows immunoblots for NR2F1, Twist and cleaved caspase 3 in mammary gland positive for tumors from control and HER2-DC1 treated BALB-neuT mice. FIG. 16E shows immunoblots for HER2 and PR protein expression in tumor cells from early lesions of control BALB-neuT mice (left) and TUBO cells (right) treated with IFN-γ, TNF-α, anti-HER2 antibodies (clones 7.16.4 and 7.9.5) or in combination. FIGS. 16F and 16G show the frequency of HER2+PCK+(16F) and HER2+PR+PCK+(16G) tumor cells in the baseline and post intratumoral HER2-DC1 treated tumors of HER2+ BC patients (n=5). All data are presented as mean±SEM represented. *, P<0.05; **, P<0.01; ***, P<0.001 by one-tailed or two-tailed Student's t test.

FIG. 17 shows the intratumoral activation of anti-tumor CD4Th1 response reduces disseminating cancer cells in HER2+ breast cancer patients, Related to FIG. 4. FIG. 17 shows mIF images of HER2, PCK, PR, RANKL, Wnt4 and DAPI in baseline and post intratumoral HER2-DC1 treated tumors of HER2+ BC patients (n=5). Scale bars, 20×.

FIGS. 18A, 18B, 18C, 18D, 18E, and 18F show the anti-tumor CD4Th1 response mediated regulation of TNBC cancer cell dissemination markers and its effects on DCCs and primary tumors, Related to FIGS. 4 and 5. FIGS. 18A, 18b, and 18C show cell surface expression of Wnt4 (18A), RANKL (18B) and Snail+Slug proteins (18C) on the tumors from control and intratumoral HER3-DC1 treated BALB/c mice bearing 4T1 tumors was analyzed by IHC. FIGS. 18d and 18E show tumors of NSG mice implanted subcutaneously with purified and equivalent numbers of DCCs from control BALB-neuT mice (18D) and remnant tissues of NSG mice implanted subcutaneously with purified and equivalent numbers of DCCs from intramammary gland HER2-DC1 treated BALB-neuT mice were stained by H&E. FIG. 18F shows tumor growth curves of NSG mice injected subcutaneously with metastatic tumor cells from control BALB/c mice and HER2-DC1 treated BALB/c mice (n=4/group). Mean±SEM represented. *, P<0.05 by two-tailed Student's t test.

FIGS. 19A, 19B, and 19C show the specificity and functional role of tumor antigen specific CD4Th1 cells, Related to FIG. 5. FIG. 19A shows senescence inducing potential of tumor antigen specific CD4Th1 cells secreting IFN-γ on TUBO cells (left) and percentage of senescent TUBO cells (right) (n=10-24). Scale bars, 4×. FIG. 19B shows tumor growth curves of BALB/c mice bearing orthotopic HER2+ TUBO tumors treated with intratumoral anti-HER2 CD4Th1 cells, intratumoral non-specific CD4Th1 cells, intratumoral HER2-DC1 or in combination (n=6-8/group). FIG. 19C shows tumor growth curves of BALB/c mice bearing subcutaneous HER2+ TUBO tumors administered intratumoral HER2-DC1 with or without intravenous injection of anti-HER2 CD4Th1 cells (n=5/group).

FIGS. 20A, 20B, 20C, 20D, 20E, 20F, 20G, and 20H show CD4Th1 cytokine IFN-γ mediated regulation of MHC class I, MHC class II and CD1d expression on early lesion and metastatic tumor cells, Related to FIG. 6. FIGS. 20A and 20B) show flow cytometry analysis for MHC class I and MHC class II expression in tumor cells from early lesion of control BALB-neuT mice (20A) and metastatic TUBO cells (20B) with or without IFN-γ treatment. FIG. 20C shows flow cytometry analysis of CD1d expression in tumor cells from early lesion of control BALB-neuT mice treated with or without IFN-γ. FIG. 20D shows the frequency of CD1d+ tumor cells from (20C) (n=3). FIG. 20E shows the mean fluorescence intensity (MFI) of CD1d expression from (20C) (n=3). FIG. 20F shows flow cytometry analysis of CD1d expression in TUBO cells treated with or without IFN-γ. FIG. 20G shows the frequency of CD1d+ tumor cells from (20F) (n=3). FIG. 20H shows the mean fluorescence intensity (MFI) of CD1d expression from (20F) (n=3). Mean±SEM represented. Means were statistically compared by two-tailed Student's t test.

FIGS. 21A, 21B, 21C, 21D, 21E, and 21F show CD4Th1 cytokine IFN-γ mediated changes on the chemokines, MHC class I and MHC class II antigen presentation-related genes in DCCs, Related to FIG. 6. FIG. 21A shows a heat map showing the differential expression of antigen presentation related genes in DCCs of BALB-neuT mice treated with or without IFN-γ. FIG. 21B shows the REACTOME analysis of upregulated chemokine and chemokine receptor related genes in IFN-γ treated DCCs of HER2+ BC patients. FIGS. 21C and 21D shows REACTOME analysis with differential expression of MHC class I antigen presentation related genes in IFN-γ treated DCCs of HER2+ breast cancer patients. FIGS. 21E and 21F shows the REACTOME analysis depicting MHC class II antigen presentation related genes differential expression in IFN-γ treated DCCs of HER2+ breast cancer patients.

FIGS. 22A, 22B, 22C, 22D, 22E, 22F, 22G, and 22H show that CD4Th1 cytokine IFN-γ inhibits cancer stemness and induces apoptosis, Related to FIG. 7. FIGS. 22A and 22B show tumor cells from early lesion of control BALB-neuT mice (22A) and TUBO cells (22B) were treated with or without IFN-γ and analyzed for ALDH+ stem cell population by flow cytometry. FIGS. 22C and 22D show flow cytometry analysis showing reduced levels of CD24+CD44+ cancer stem cell population in tumor cells from early lesion of control BALB-neuT mice (22C) and TUBO cells (22D). FIG. 22E shows Annexin V⁺7-AAD⁺ apoptotic DCCs of BALB-neuT mice treated with IFN-γ in combination with anti-HER2 antibodies (7.16.4 and 7.9.5). FIGS. 22F and 22G show Annexin V⁺7-AAD⁺ apoptotic population in tumor cells from early lesions of control BALB-neuT mice (22F) and TUBO cells (22G) treated with IFN-γ or in combination with anti-HER2 antibodies (7.16.4 and 7.9.5). FIG. 22H shows a heat map showing differential expressions of apoptosis-related genes in IFN-γ treated DCCs of HER2+ BC patients.

FIGS. 23A, 23b, 23C, and 23D show that CD4Th1 cytokine IFN-γ regulates various signaling pathways in DCCs, Related to FIG. 8, FIGS. 23A and 23D show protein pathway enrichment analysis for the downregulated (23A) and upregulated pathways (23B) for datasets in IFN-γ treated DCCs isolated from BALB-neuT mice. FIGS. 23C and 23D show protein pathway enrichment analysis depicting downregulated (23C) and enriched pathways (23D) for the differentially expressed datasets in IFN-γ treated DCCs from HER2+ breast cancer patients.

FIG. 24 shows that CD4Th1 cytokine IFN-γ regulates ISDS signature genes in JIMT-1, Related to FIG. 8, in a heat map showing the differential expression of ISDS genes associated with good prognosis in JIMT-1 cells after IFN-γ treatment.

### VII. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### 1. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “immunotherapy” and “immunotherapeutic” refer to the treatment of disease by activating or suppressing the immune system. In cancer treatment, the most effective immunotherapies are cell-based immunotherapies that utilize lymphocytes, macrophages, dendritic cells, natural killer cells, cytotoxic T lymphocytes, etc. to defend the body against cancer by targeting abnormal antigens expressed on the surface of tumor cells.

“Hormone therapy” or “endocrine therapy” refers to a cancer treatment that slows or stops the growth of cancer (such as, for example, prostate cancer or breast cancer) that uses hormones to grow. It is most often used along with other anti-cancer treatments such as, for example Immunotherapy, chemotherapy, surgery, or radiation therapy.

“Targeted therapy” refers to a type of cancer treatment that uses a drug, substance, composition, or formula that precisely identifies and targets proteins that control how cancer cells grow, divide, and spread and then attacks/kills the cancer cell. A targeted therapy can be used by itself or in combination with other anti-cancer treatments, such as, for example chemotherapy, surgery, or radiation therapy.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### 2. Methods of Detecting Disseminated Cancer Cells

Immunosuppressive tumor microenvironment (TME) can help cancer cell dissemination, evade immune surveillance, and give rise to metastatic colonization of DCCs. Myeloid-derived suppressor cells (MDSCs) can suppress anti-tumor immunity and promote metastasis progress of DCCs in 4T1 tumor bearing mice, indicating their role in creating pre-metastatic niches. Macrophages have been reported to induce HER2 early cancer cell dissemination and support DCCs mediated metastasis development in MMTV-HER2 model. Although, CD8 T cells were able to clear DCCs in distant organs and prevent metastasis in EMT6 tumor bearing mice model, it failed to induce such effects in 4T1 tumor models. Furthermore, neutrophils have been identified to suppress natural killer (NK) cells mediated clearance of DCCs and enhance metastasis formation. Thus, manipulating immunosuppressive cascades can be an effective strategy to eradicate DCCs and prevent metastasis.

Type 1 conventional dendritic cells (cDCT) can prime CD4 Th1 cells and generate tumor antigen specific anti-tumor immunity in cancers. However, loss of anti-tumor CD4Th1 immunity was observed to negatively impact treatment responses and prognosis in HER2 positive and triple negative breast cancer patients. Delivery of tumor antigen pulsed DCT was able to induce pathologic complete responses (pCR) and recurrence free survival in HER2+ early-stage breast cancer patients. Notably, intratumoral delivery of tumor antigen pulsed DC1 combined with targeted antibodies induced systemic immunity and eradicated both primary and metastatic tumors by the activation of CD4 T, CD8 T, B and NK cells and inhibition of MDSCs in HER2 driven mouse mammary tumor models. Interferon gamma (IFN-γ) is a primary effector cytokine of anti-tumor CD4 Th1 cells can inhibit growth and spread of cancers. IFN-γ can also enhance the function of CD4 T, CD8 T, and NK cells and DCs, leading to tumors clearance. Despite their prominent contribution in targeting primary tumors, prior to the present disclosure the role of tumor-antigen specific CD4 Th1 cells on DCCs was not known in breast cancer.

Herein, we provide evidence that cDC1 primed tumor antigen specific CD4 T cells inhibit cancer cell dissemination via regulating dissemination mechanisms and eradicates DCCs in distant organs and inhibit tumorigenic and metastasis growing potential of DCCs. Our results also demonstrate that CD4Th1 cytokine IFN-γ secretion has the ability to alter various molecular gene signatures, prevent stemness and metastasis growth potential of DCCs from breast cancer patients.

Also disclosed herein are methods of detecting the presence of disseminated cancer cells (DCC) in the subject, the method comprising obtaining a tissue sample (such as, for example, blood, bone marrow, or tissue biopsy) from the subject, and further performing immunomagnetic enrichment of EPCAM+ DCCs in blood and bone marrow aspirates or immunohistochemical staining of tissue biopsies or a Prelude DCIS identification assay.

In one aspect, disclosed herein are methods of treating, reducing, decreasing, and/or inhibiting cancer cell dissemination and/or metastasis in a subject with a cancer (such as, for example, breast cancer, including, but not limited to triple negative breast cancer) comprising administering to the subject (systemically or intratumorally) tumor antigen (such as, for example an oncodriver including, but not limited to human epidermal growth factor receptor (HER) 1(HER1), HER2, HER3, epidermal growth factor receptor (EGFR), c-Mesenchymal to Epithelial Transition (c-MET), B-Rapidly Accelerated Fibrosarcoma (BRAF), KIT, Androgen Receptor (AR), Estrogen Receptor (ER), Kirsten rat sarcoma (KRAS), TP53, or APC)-pulsed Type 1 conventional dendritic cells (cDC1). Examples of such epitopes can be found in Table 1 and 2 below.

TABLE 1
HER3 CD4+ T cell epitopes

| | | Homology with |
|---|---|---|
| HER3 Epitopes Identified | Amino Acid Sequences | Mouse HER3 |
| HER3 ECD P12 (aa 57-71) | EVVMGNLEIVLTGHN (SEQ ID NO: 1) | 100% |
| HER3 ECD P81 (aa 401-415) | SWPPHMHNFSVFSNL (SEQ ID NO: 2) | 100% |
| HER3 ECD P84 (aa 416-430) | TTIGGRSLYNRGESL (SEQ ID NO: 3) | 100% |
| HER3 ECD P91 (aa 450-464) | SAGRIYISANRQLCY (SEQ ID NO: 4) | 87% |
| HER3 ICD P38 (aa 850-864) | VADFGVADLLPPDDK (SEQ ID NO: 5) | 100% |
| HER3 ICD P41 (aa 865-879) | QLLYSEAKTPIKWMA (SEQ ID NO: 6) | 93% |
| HER3 ICD P52 (aa 920-934) | VPDLLEKGERLAQPQ (SEQ ID NO: 7) | 93% |
| HER3 ECD P12 (aa 56-70) | CEVVMGNLEIVLTGH (SEQ ID NO: 8) | |
| HER3 ECD P91 (aa 451-465) | AGRIYISANRQLCYH (SEQ ID NO: 9) | |
| HER3 ICD P86 (aa 1090-1114) | GCLASESSEGHVTGS (SEQ ID NO: 10) | |
| HER3 ICD P89 (aa 1115-1129) | EAELQEKVSMCRSRS (SEQ ID NO: 11) | |

TABLE 2

| HER2 Epitoped | |
|---|---|
| Identified | Amino Acid Sequence |
| HER2 P42-56 | HLDMLRHLYQGCQVV |
| | (SEQ ID NO: 12) |
| HER2 P98-114 | RLRIVRGTQLFEDNYAL |
| | (SEQ ID NO: 13) |
| HER2 P328-345 | TQRCEKCSKPCARVCYGL |
| | (SEQ ID NO: 14) |
| HER2 P776-790 | GVGSPYVSRLLGICL |
| | (SEQ ID NO: 15) |
| HER2 P927-941 | PAREIPDLLEKGERL |
| | (SEQ ID NO: 16) |
| HER2 P1166-1180 | TLERPKTLSPGKNGV |
| | (SEQ ID NO: 17) |

In some aspects, rather than administering pulsed dendritic cells directly to the subject, CD4 T cells can be cultured with the pulsed dendritic cells ex vivo and the CD4 T cells administered to the subject. Accordingly, disclosed herein are methods of treating, reducing, inhibiting, and/or decreasing cancer cell dissemination and/or metastasis in a subject with a cancer (such as, for example, breast cancer, including, but not limited to triple negative breast cancer) comprising obtaining TH1 CD4 T cells; contacting the CD4 T cells with tumor antigen (such as, for example an oncodriver including, but not limited to human epidermal growth factor receptor (HER) 1(HER1), HER2, HER3, epidermal growth factor receptor (EGFR), c-Mesenchymal to Epithelial Transition (c-MET), B-Rapidly Accelerated Fibrosarcoma (BRAF), KIT, Androgen Receptor (AR), Estrogen Receptor (ER), Kirsten rat sarcoma (KRAS), TP53, or APC)-pulsed Type 1 conventional dendritic cells thereby creating DC1 primed CD4 T cells; and administering (systemically or intratumorally) to the subject the DC1 primed CD4 T cells. Examples of such epitopes can be found in Table 1 and 2 above.

In one aspect disclosed herein are methods of treating, reducing, decreasing, and/or inhibiting cancer cell dissemination and/or metastasis, further comprising detecting the presence of disseminated cancer cells (DCC) in the subject, the method comprising obtaining a tissue sample (such as, for example, blood, bone marrow, or tissue biopsy) from the subject, performing immunomagnetic enrichment of EPCAM+ DCCs in blood and bone marrow aspirates or immunohistochemical staining of tissue biopsies or a Prelude DCIS identification assay.

The disclosed compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer (including triple negative breast cancer (TNBC), metastatic breast cancer (MBC), ductal carcinoma in situ (DCIS)); genitourinary cancer; pulmonary cancer; esophageal carcinoma; head and neck carcinoma; large bowel cancer; hematopoietic cancers; testicular cancer; and colon and rectal cancers.

As shown herein, the reduction in disseminated cancer cells (DCCs) can have an abscopal effect (i.e., therapeutic effect and reduction of cancers at sites biologically distant from the targeted cancer). Accordingly, in some aspects, the methods of reducing DCCs and/or treating metastasis disclosed herein can further treat, reduce, decrease, and/or inhibit cancers at sites distant from the tumor microenvironment of the cancer being targeted for treatment (abscopal effect).

It is understood and herein contemplated that while a single administration of the components of the disclosed anti-cancer therapies (i.e., the pulsed dendritic cells and/or CD4 T cells cultured with said pulsed dendritic cells) would be ideal, not every patient will respond in the same manner. Thus, in one aspect, disclosed herein are anti-cancer therapies methods treating, preventing, reducing, and/or inhibiting a DCCs or metastasis; wherein the at least one pulsed dendritic cell or CD4 T cell is administered at least 1, 2, 3, 4, 5, 6, 7, 8, 9, 10, 11, 12, 13, 14, 15, 16, 17, 18, 19, 20, 21, 22, 23, or 24 times per day or at least 1, 2, 3, 4, 5, 6, 7, 8, 9, 10, 11, 12, 13, or 14 times per week for at least 1, 2, 3, 4, 5, 6, 7, 8, 9, 10, 11, 12, 13, 14 days, 3, 4, 5, 6, 7, 8, 9, 10, 11, 12 weeks. It is further understood and herein contemplated that the order and duration of the administered components can vary as appropriate for the subject being treated. In one aspect, disclosed herein are anti-cancer therapies methods treating, preventing, reducing, and/or inhibiting a DCCs or metastasis; wherein the pulsed dendritic cells and/or CD4 T cells are administered at least 1, 2, 3, 4, 5, 6, 7, 8, 9, 10, 11, 12, 18, 24, 30, 36 hours, 2, 3, 4, 5, 6, 7, 8, 9, 10, 11, 12, 13, 14, 21, 28, 30, 31, 45 days, 2, 3, 4, 5, or 6 months.

In one aspect, it is understood and herein contemplated that the pulsed dendritic cells can be activated prior to administration as well as prior to being pulsed with the oncodriver. Activation of the dendritic cells (DC1) can be achieved by contacting the cells with IFN-γ, TNFα, CD40, IL21, and/or IL-12. Accordingly, disclosed herein are methods of treating, reducing, decreasing, and/or inhibiting cancer cell dissemination and/or metastasis, wherein the tumor antigen CD4+ T cell epitope pulsed dendritic cell is activated with IL-12 prior to administration.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pemlbroiizumab, CT-011, MK-3475), PD-L1 (such as, for example, aezoliunmab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep).

### 1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### 3. Examples

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Anti-Tumor CD4Th1 Response Target and Induce Immune Recognition of Disseminated Cancer Cells and Prevent Overt Metastasis

a. Introduction

Metastatic spread in breast cancer (BC) patients is the major driver of cancer-related death. It is widely believed that dissemination of cancer cells (DCC) from a clinically latent stage of invasive breast tumors manifests metastasis. However, a large cohort of BC studies have demonstrated that pre-invasive and primary tumors also release DCCs as an early hidden event, potentially forming metastases following successful treatment of the primary tumor. The presence of DCCs in the bone marrow (BM) of patients with early-stage disease is a significant predictor of subsequent local recurrence and development of metastases. Studies from spontaneous mouse BC models have demonstrated early DCCs can reach distant organs and then proliferate to form metastasis. DCCs remain dormant for prolonged periods of time before developing clinically apparent metastasis in patients. DCCs express gene signatures that differ from circulating tumor cells. The heterogeneity and stemness properties of DCCs can promote their resistance to adjuvant or neoadjuvant therapies and may cause cancer recurrence and incurable metastasis.

The arrival of DCCs in the host BM and other distant organs initiates complex eco-evolutionary dynamics often characterized as a “seed and soil” interactions. The DCCs (seed) evade multiple elements of the immune system that can engage in predator-like attacks on the DCCs and successfully occupy the target organs (soil) to create a pre-metastatic niche. An immunosuppressive tumor microenvironment (TME) in the target organs can permit DCCs to evade immune surveillance, render dormancy and then proliferate to form metastases. A granulocytic subset of myeloid-derived suppressor cells (gMDSCs) has been shown to suppress anti-tumor immunity and promote metastasis of DCCs. Similarly, macrophages have been reported to induce early cancer cell dissemination and DCC-mediated metastasis development. In addition, neutrophils can suppress natural killer (NK) cell-mediated clearance of DCCs and enhance metastasis formation. The balance of the protumorigenic niche for DCCs is maintained by anti-tumor immune mechanisms. Cytotoxic CD8 T cells can clear DCCs in distant organs and prevent metastasis in some, but not in all tumor models. Priming of CD4Th1 cells during the early stage of tumor progression is critical and these cells reinforce targeted therapy to completely eradicate tumors. Nevertheless, the role for CD4Th1 cells on DCCs is unknown, and this study addresses if these cells can modulate DCCs.

### b) Results

1. Gene Expression Profiles of DCCs is Different than Primary and Metastatic Tumors

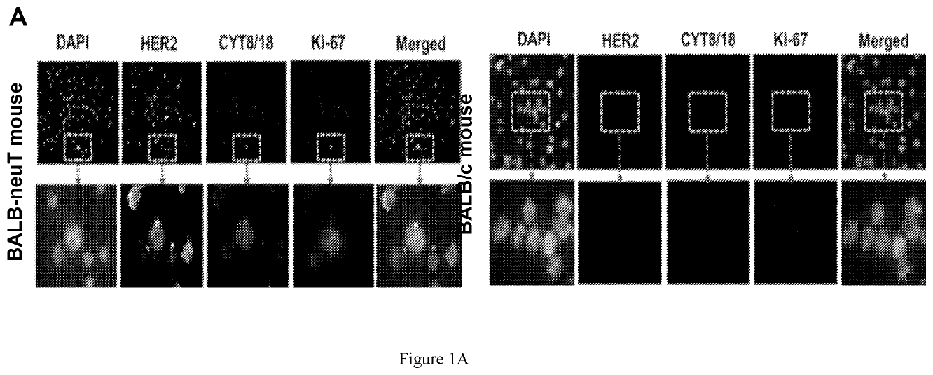

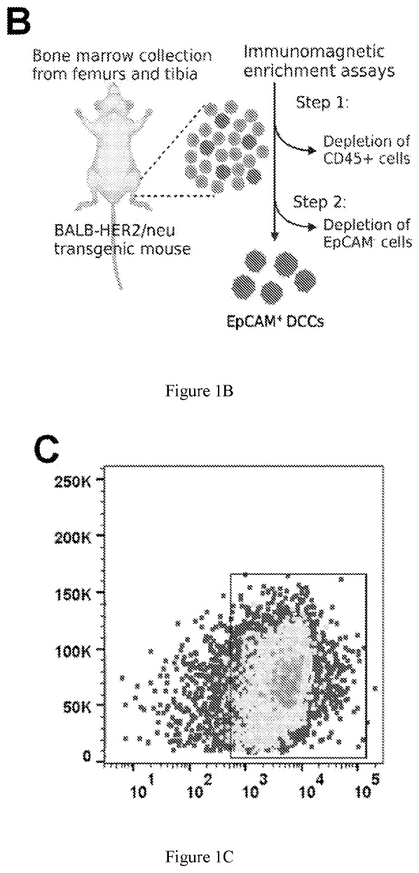

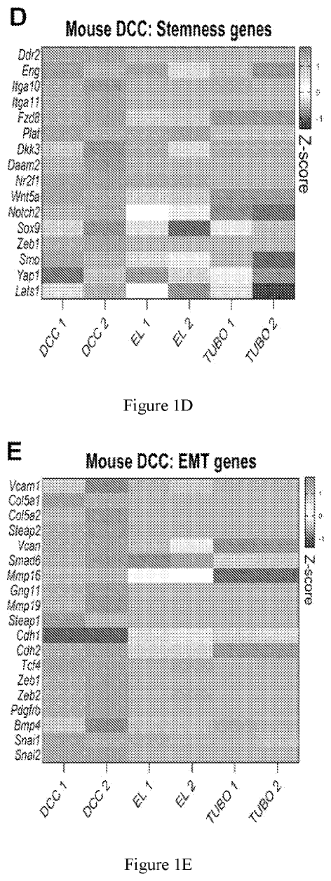

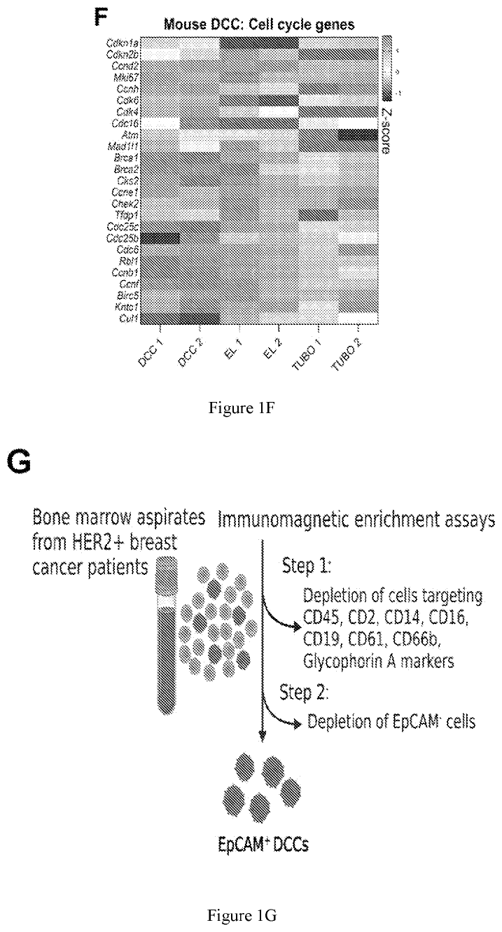

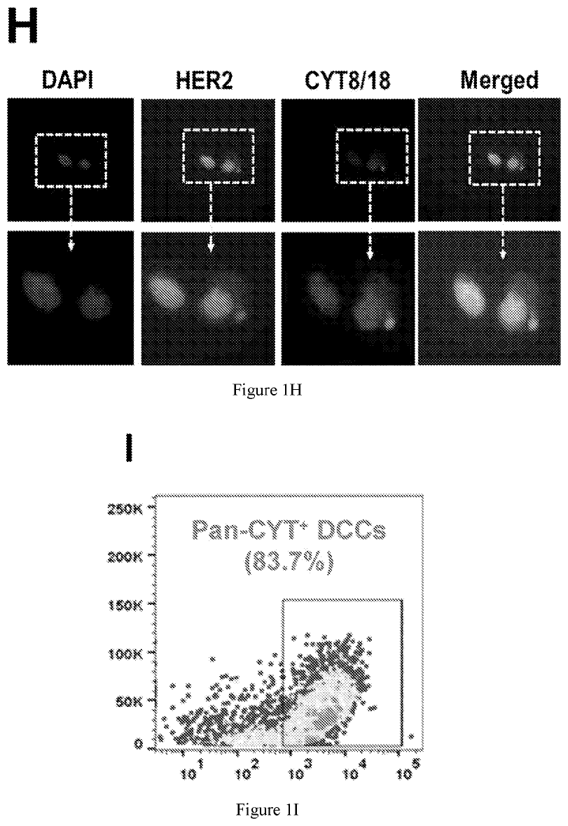

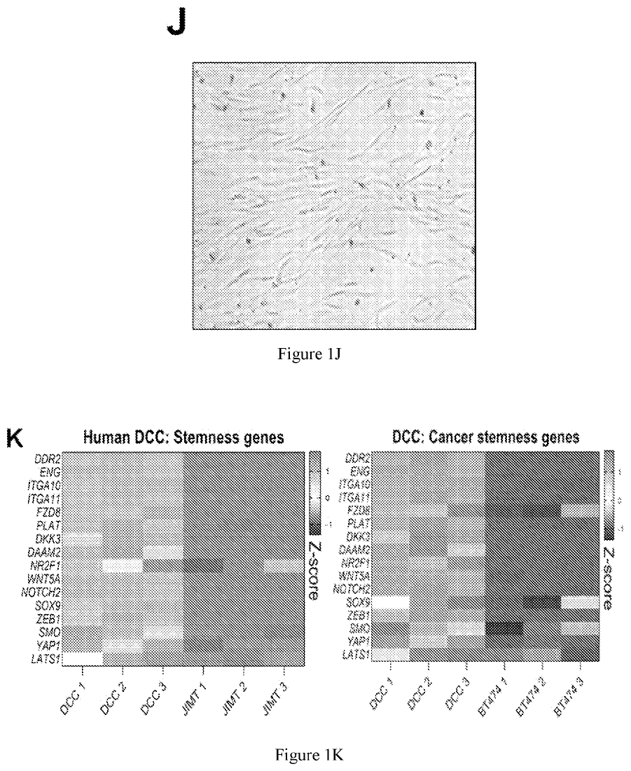

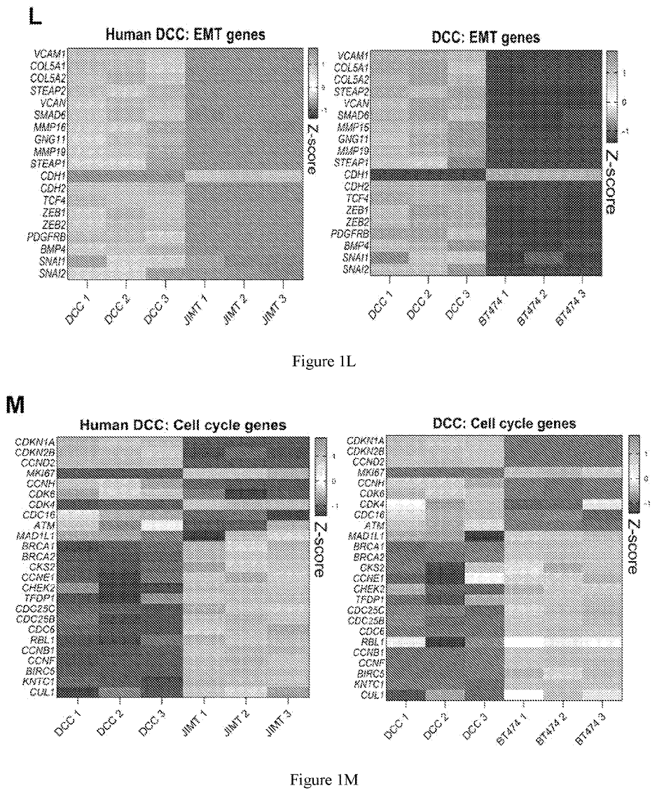

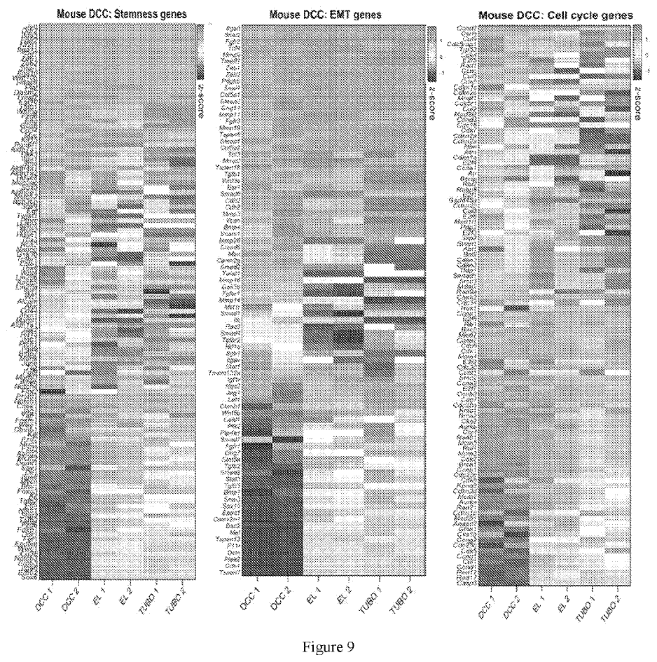

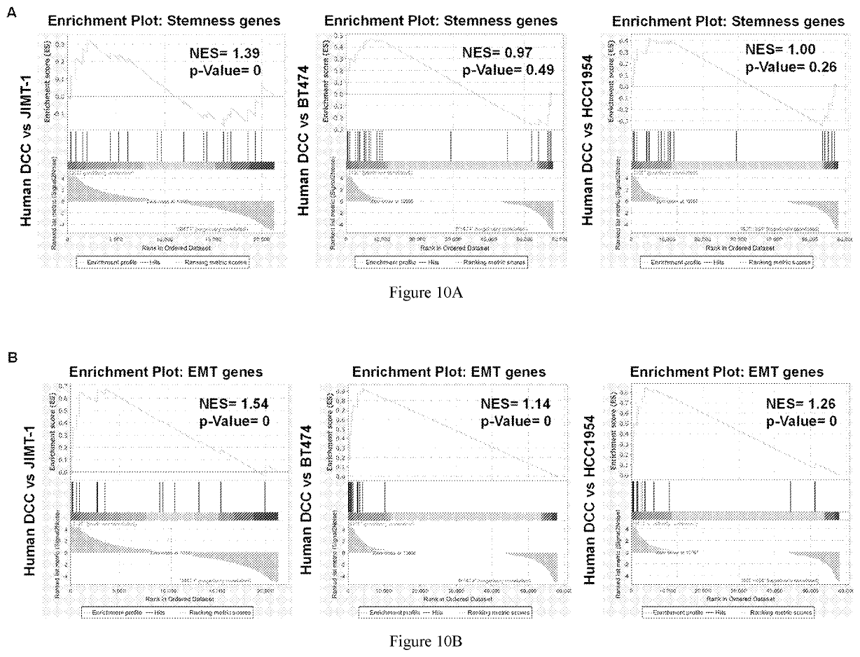

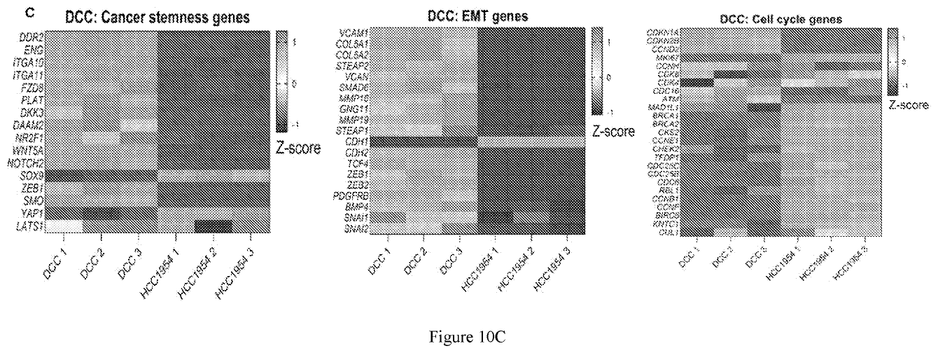

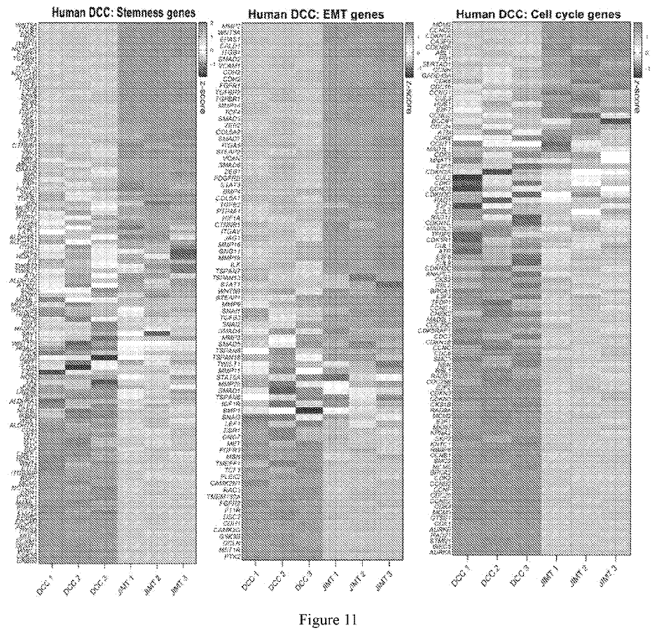

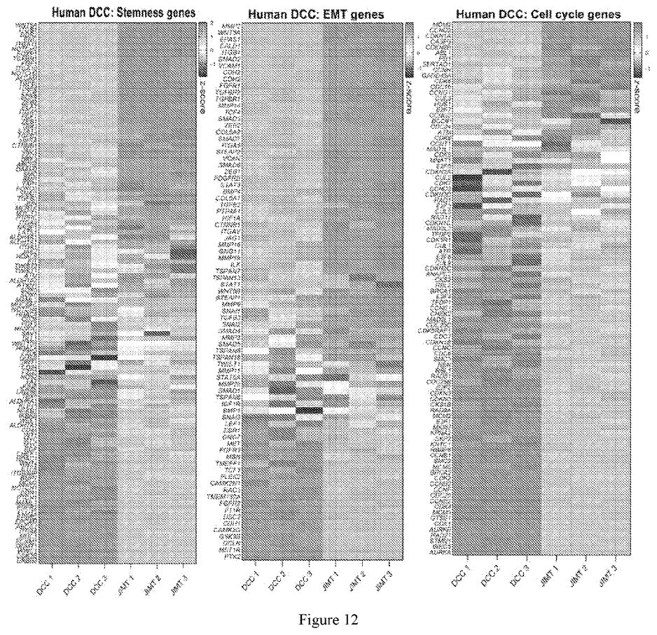

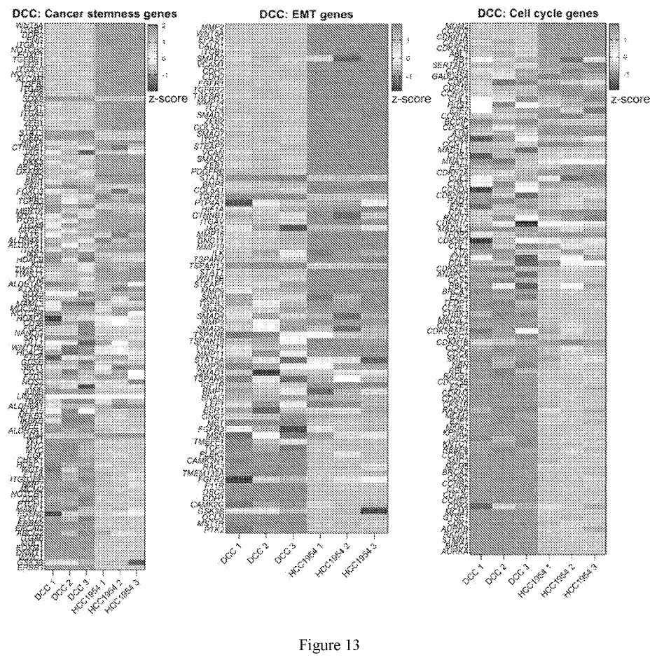

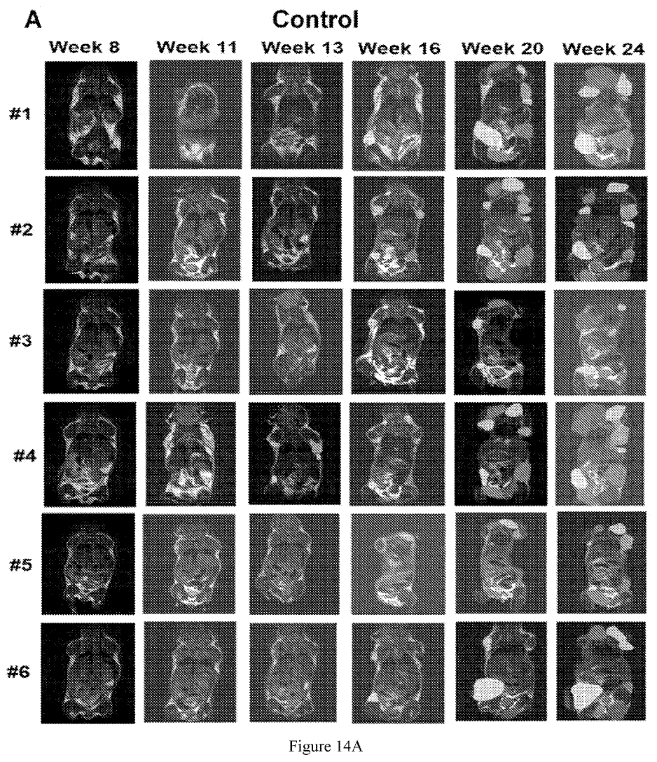

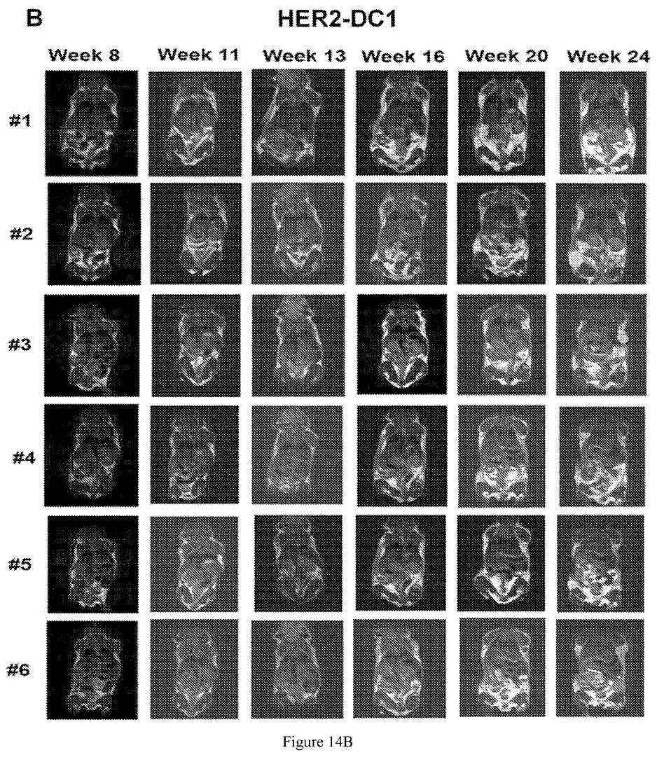

The presence of DCCs was observed in the BM of BALB-neuT mice with early lesion mammary carcinoma (FIG. 1A). This was consistent with the previous findings. Notably, there were no detectable DCCs in the BM of naïve BALB/c mice (FIG. 1A). The EpCAM⁺ DCCs from the BM of BALB-neuT mice was isolated by utilizing a two-step immunomagnetic enrichment assay and isolation purity was verified by flow cytometry (FIGS. 1B and 1C). The gene expression profiles of DCCs from BALB-neuT mice was analyzed by RNA sequencing and revealed upregulation of various sternness and epithelial to mesenchymal transition (EMT) genes and downregulation of cell cycle genes compared to early lesion tumor cells of BALB-neuT mice and metastatic TUBO cells (FIGS. 1D-1F and 9). Next, the BM aspirates from HER2+ ductal carcinoma in situ (DCIS) and early invasive BC patients (prior to any treatment) obtained at the time of surgery was analyzed by two step immunomagnetic enrichment assays to isolate EpCAM+ DCCs (FIG. 1G). The presence of DCCs in the BM of HER2+ BC patients and isolation purity were confirmed by immunofluorescence staining and flow cytometry (FIGS. 1H and 1I). These isolated DCCs were cultured and expanded in appropriate culturing conditions (FIG. 1J). The gene expression profiles of isolated DCCs from HER2′+ BC patients were examined by RNA sequencing. Strikingly, the DCCs of HER2+ BC patients had similar gene expression patterns for cancer stemness, EMT and cell cycle observed in DCCs of BALB-neuT mice, when compared to human HER2+ BC cells such as JIMT-1 (grade 3 invasive, T2N1M0), BT474 (invasive) and HCC1954 (stage IIA, grade 3 invasive) (FIGS. 1K-1M and 10-13). The Gene Set Enrichment Analysis (GSEA) also revealed enrichment of cancer sternness and EMT gene signatures in DCCs of HER2+ BC patients compared to JIMT-1, BT474 and HCC1954 cells (FIGS. 10A and 10B). Taken together, these data indicate that DCCs display different gene expression profiles than primary and metastatic tumors in BC.

### 2. Intratumoral Priming of Anti-Tumor CD4Th1 Immune Response Prevents Metastasis

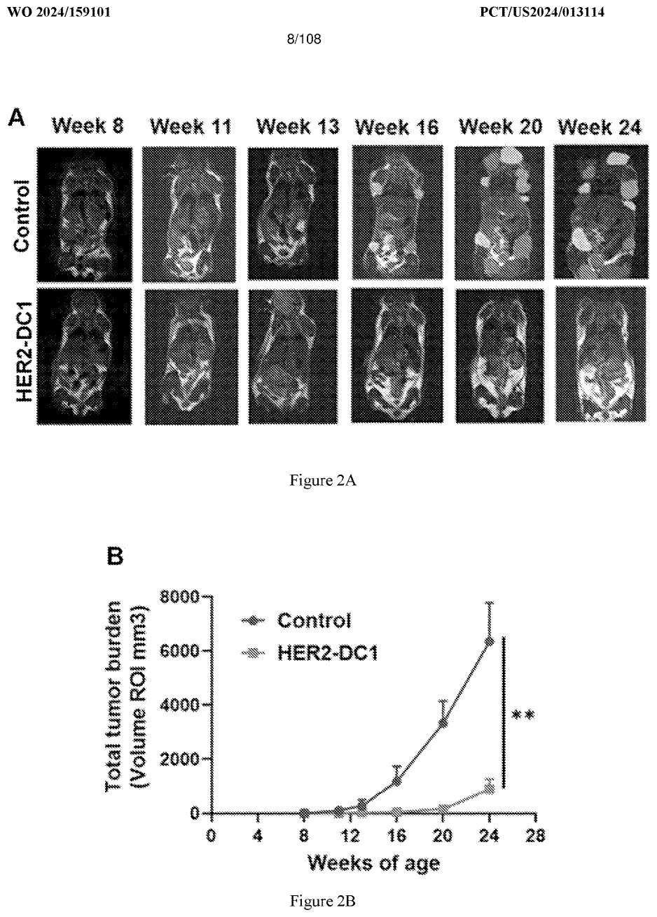

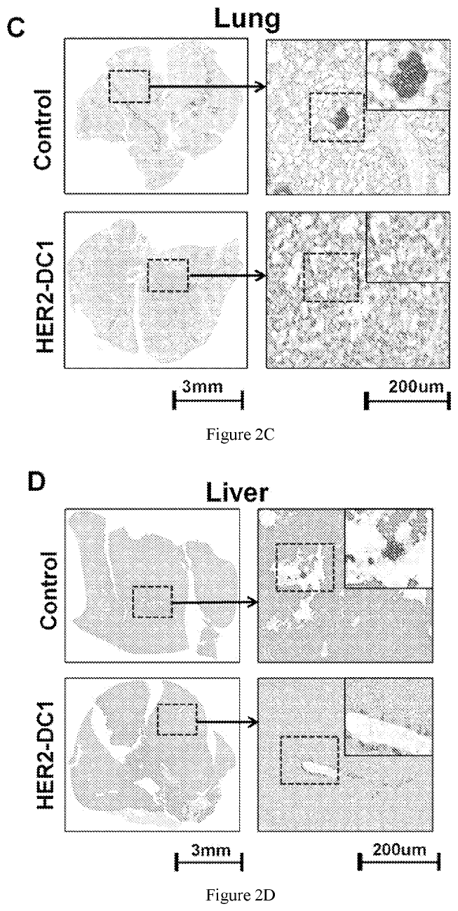

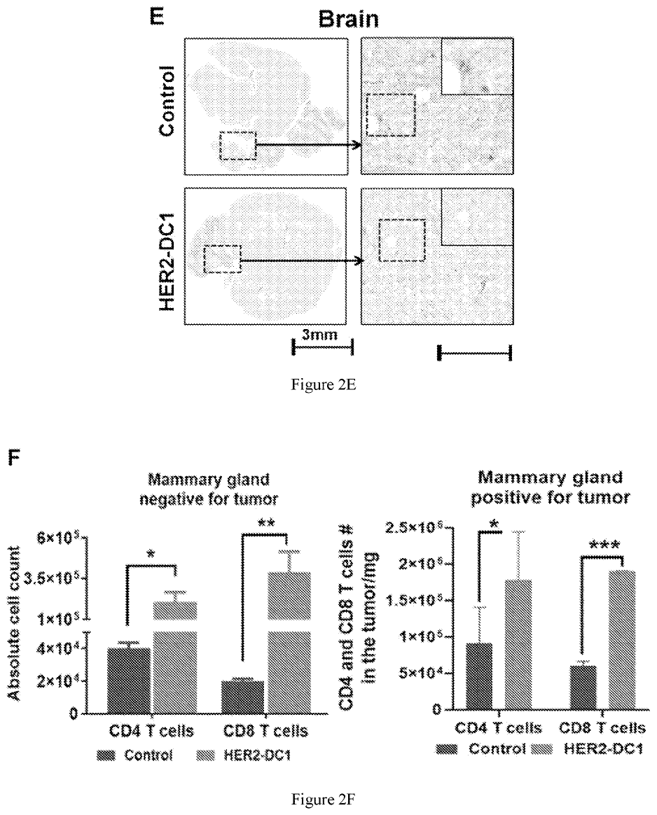

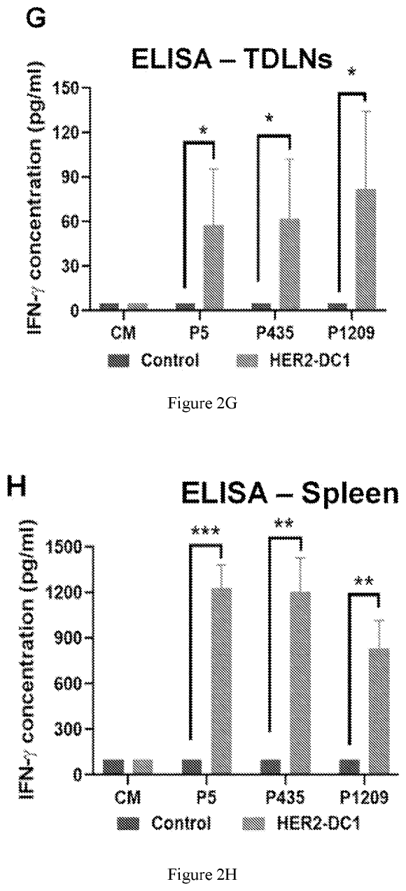

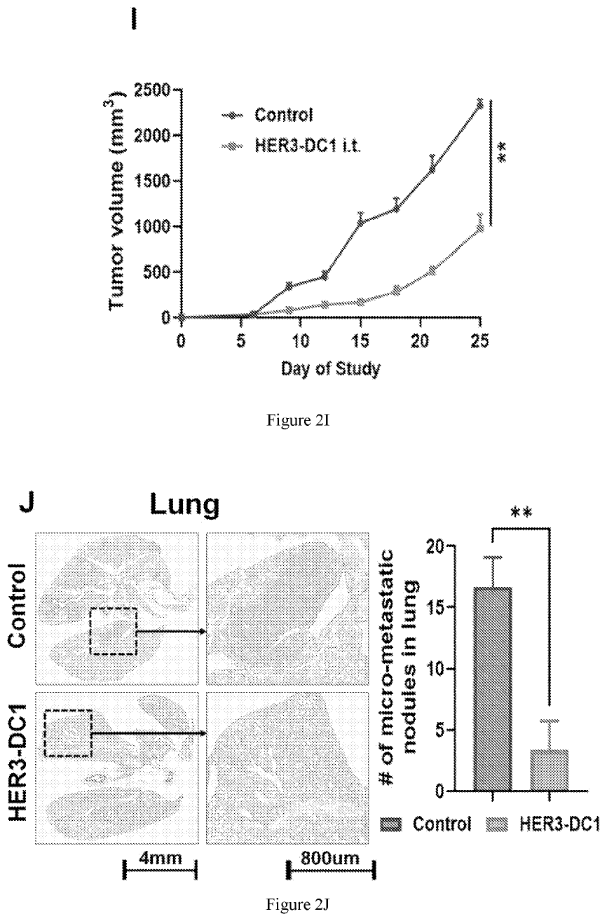

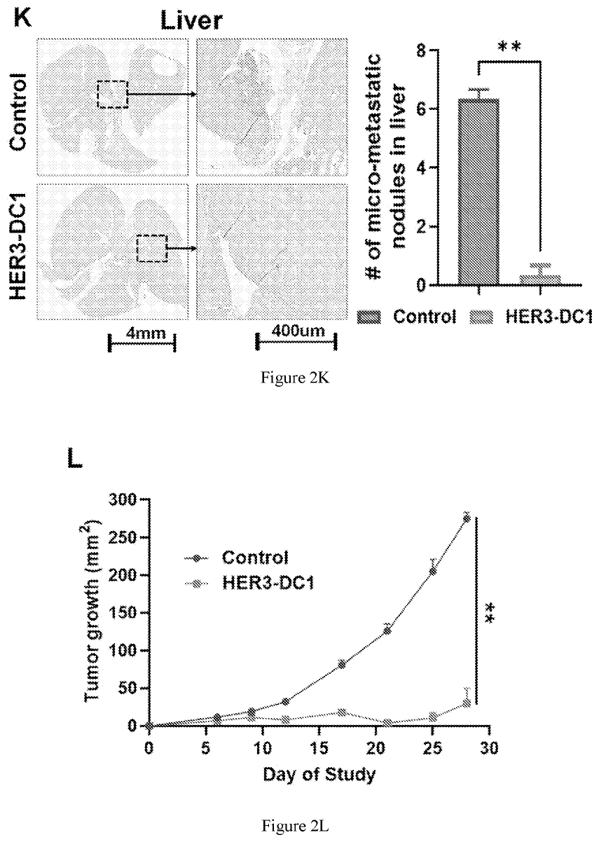

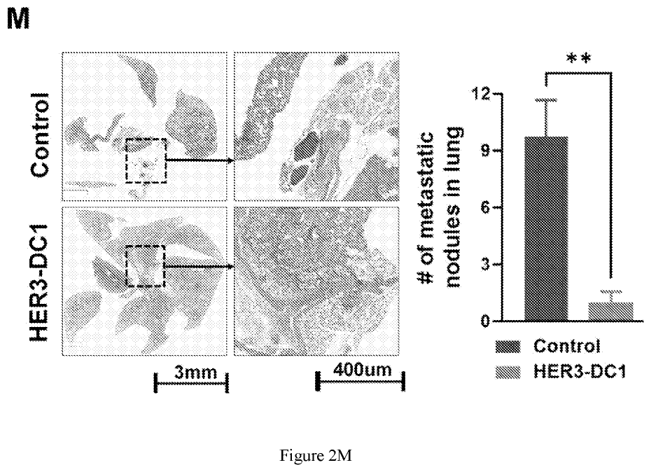

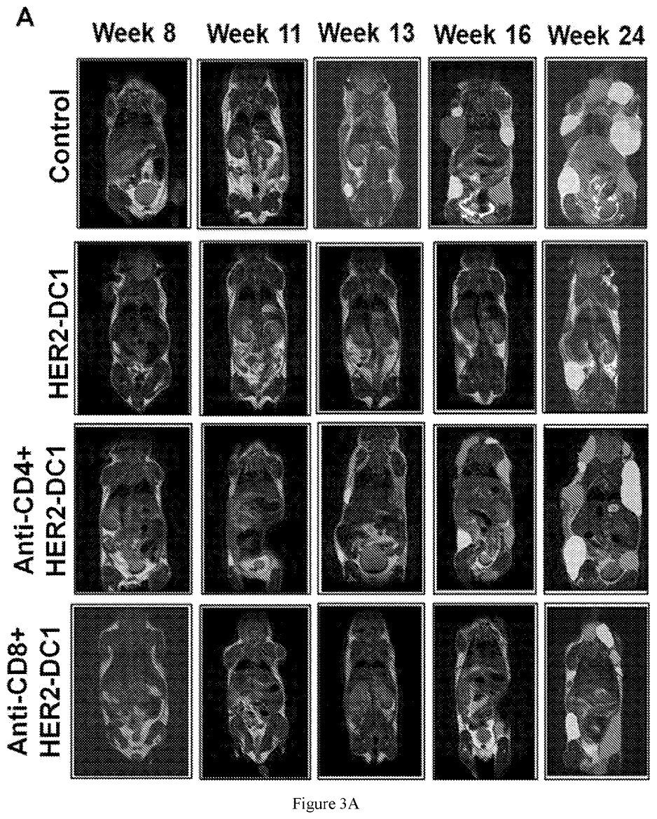

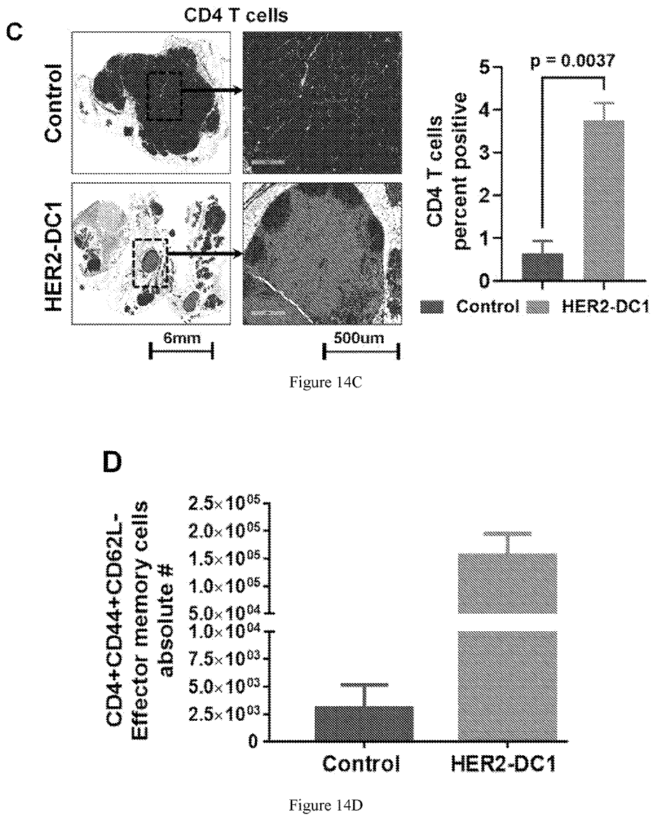

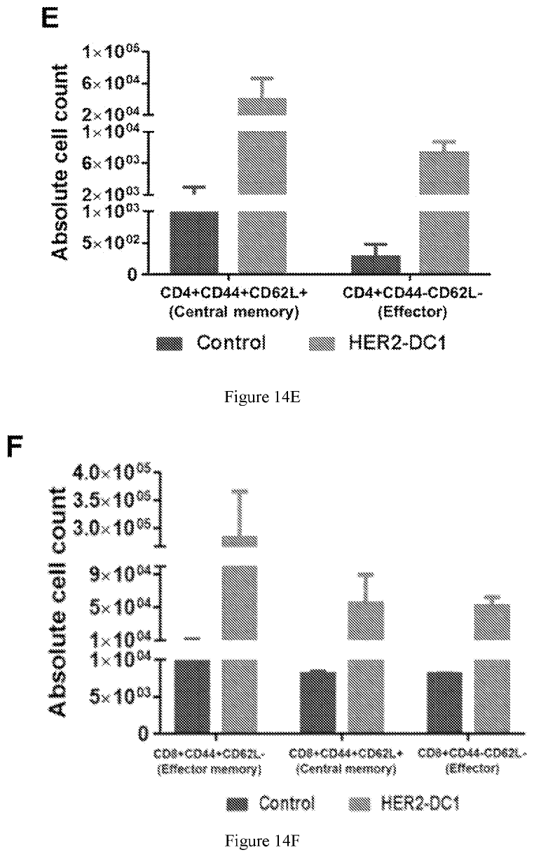

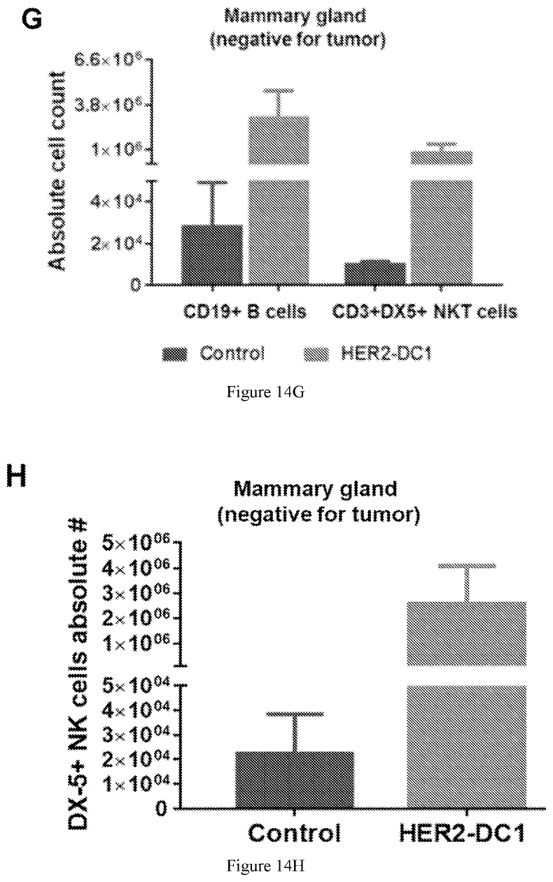

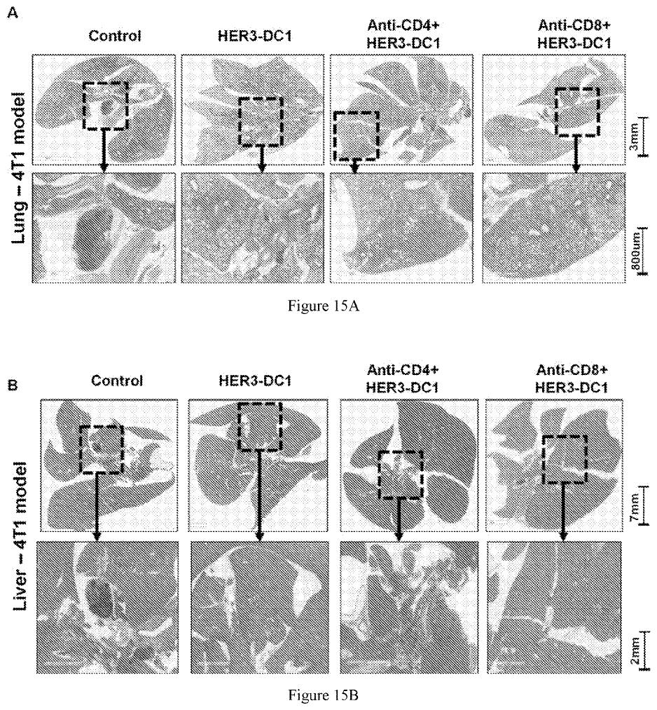

The efficacy of intramammary gland delivery was investigated of HER2-DC1 in a BALB-neuT mouse model. These mice spontaneously develop micro-invasion from 8-9 weeks of age which slowly progress from pre-invasive lesions to primary invasive tumors in all ten mammary glands and develop distant metastasis. Type 1 conventional dendritic cells (cDC1) were pulsed with MHC class II recognizing immunogenic HER2/neu peptides to prime HER2 tumor antigen specific CD4Th1 response. BALB-neuT mice at 8-9 weeks of age (before onset of primary tumors) received weekly ultrasound-guided intramammary gland injection of HER2-DC1 into their fifth mammary gland for six weeks. Severe spontaneous mammary carcinoma progression was observed in control BALB-neuT mice and reached endpoint at 22-24 weeks of age (FIGS. 2A, 2B, and 14A). HER2-DCT delivery significantly delayed spontaneous tumor growth, reduced disease severity, and improved survival of BALB-neuT mice (FIGS. 2A, 2B, and 14B). Micro-metastasis in lung, liver, and brain of control BALB-neuT mice at 24 weeks of age was observed (FIGS. 2C-2E). Interestingly, no evidence for micro-metastases was observed in these organs collected from HER2-DC1 treated BALB-neuT mice at 24 weeks of age (FIGS. 2C-2E).

Next, the level of CD4 T and CD8 T cell infiltration was examined by flow cytometry. A significant increase in the level of CD4 T and CD8 T cells was observed in mammary glands both negative and positive for tumors in HER2-DC1 treated BALB-neuT mice compared to the control group at 16-18 weeks of age (FIG. 2F). High intratumoral infiltration of CD4 T cells after HER2-DCT delivery in BALB-neuT mice was further confirmed by immunohistochemistry (FIG. 14C). Flow cytometry based immune profiling also revealed an increase in CD4⁺CD44⁺CD62L⁻ or CD8⁺CD44⁺CD62L effector memory, CD4⁺CD44⁺CD62L⁺ or CD8⁺CD44⁺CD62L⁺ central memory, CD4⁺CD44⁻CD62L⁻ or CD8⁺CD44⁻CD62L⁻ effector cells, B cells, NK and NKT cells infiltration in mammary glands negative for the tumors of HER2-DC1 treated BALB-neuT mice as compared with controls (FIGS. 14D-14H). The anti-HER2 Th1 immune response was evaluated in both control and HER2-DC1 treated BALB-neuT mice by co-culturing tumor draining lymph nodes (TDLNs) with HER2-DC1 pulsed with MHC class II HER2 peptides p5, p435 and p1209 individually. Significantly increased IFN-γ secretion was observed after co-culturing HER2-DC1 with TDLNs from the HER2-DC1 treated BALB-neuT mice, compared to control group (FIG. 2G). In addition, restimulation of splenocytes from the HER2-DC1 treated BALB-neuT mice with HER2 peptides p5, p435 and p1209 showed increased IFN-γ secretion compared to control (FIG. 2H).

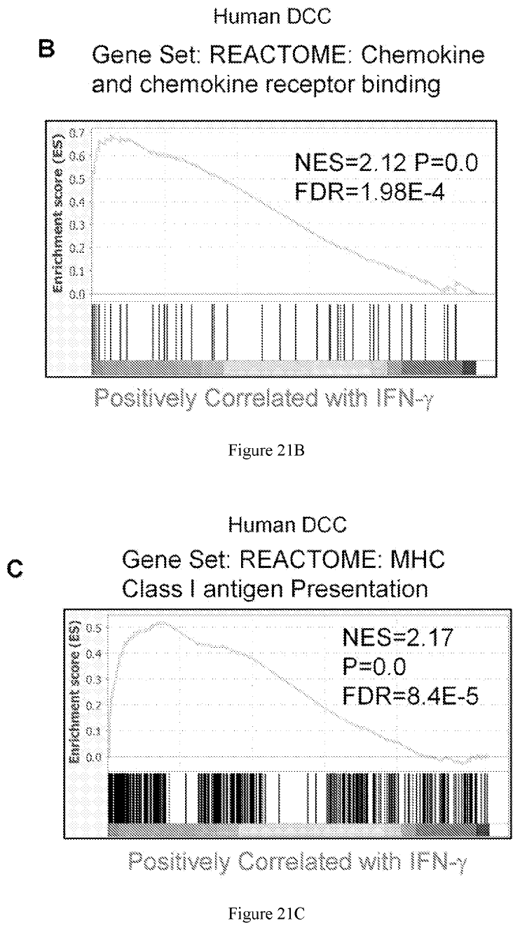

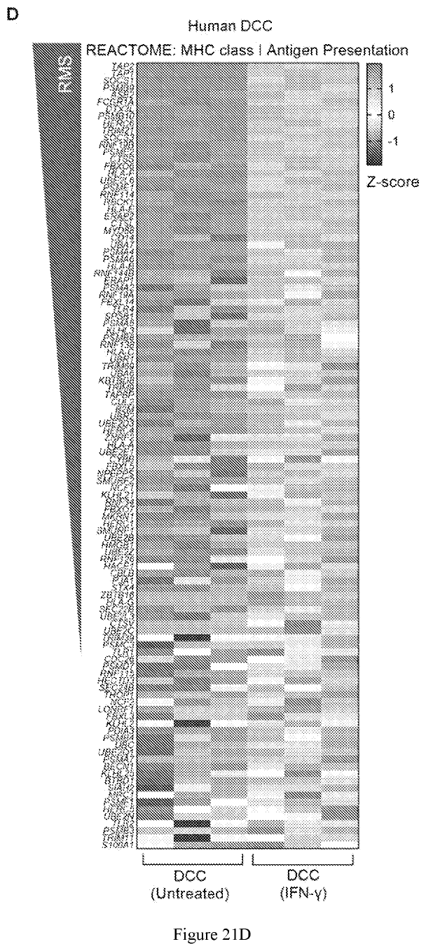

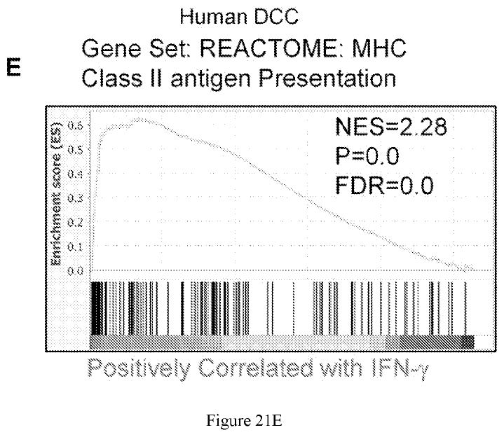

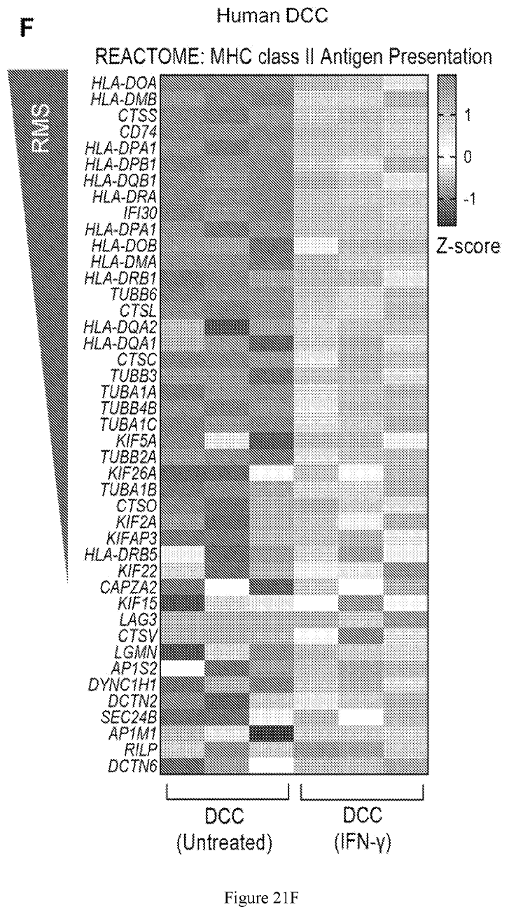

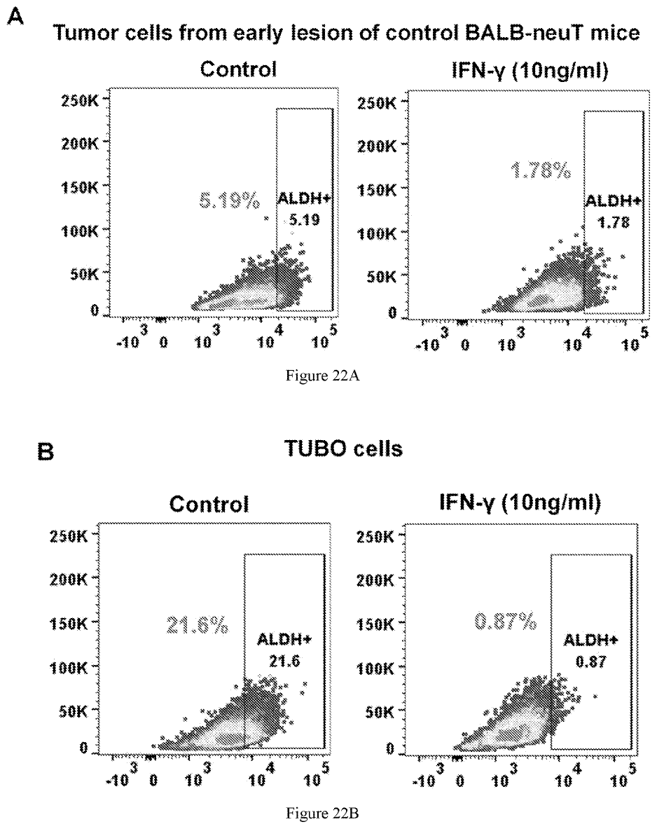

The efficacy of tumor antigen pulsed DC1 delivery on the spontaneous metastatic growth in the 4T1 tumor model was examined. The 4T1 tumor model mimics the human triple negative breast cancer (TNBC) and has been shown to overexpress HER3 tumor antigen. Overexpression of HER3 in TNBC promotes tumor growth, aggressiveness and metastasis and is associated with poor survival in patients. cDC1 were pulsed with MHC class II recognizing immunogenic HER3 peptides to prime anti-HER3 CD4Th1 response. Intratumoral delivery of cDC1 targeting the HER3 (HER3-DC1) was able to control orthotopically implanted 4T1 primary tumor growth in the mammary gland of BALB/c mice (FIG. 2I). Interestingly, HER3-DC1 delivery significantly inhibited spontaneous lung and liver metastasis in these mice compared to untreated control orthotopic 4T1 tumor bearing mice (FIGS. 21 and 2K). Next, the study was extended in a HER3 overexpressing B16-F10 melanoma spontaneous metastasis model and observed that intratumoral HER3-DC1 delivery significantly inhibited primary melanoma growth and spontaneous lung metastasis in C57BL/6 mice (FIGS. 2L and 2M). Collectively, these results suggest that cDC1 intratumoral delivery primes tumor antigen specific anti-tumor CD4Th1 immunity, which only modestly controlled primary tumor growth but significantly inhibited metastasis.

### 3. Metastatic Preventive Potential is Dependent on Anti-Tumor CD4Th1 Cells

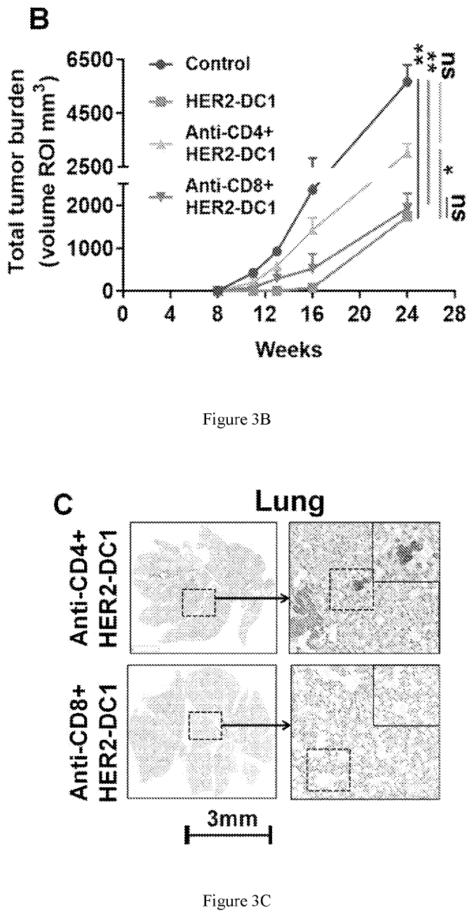

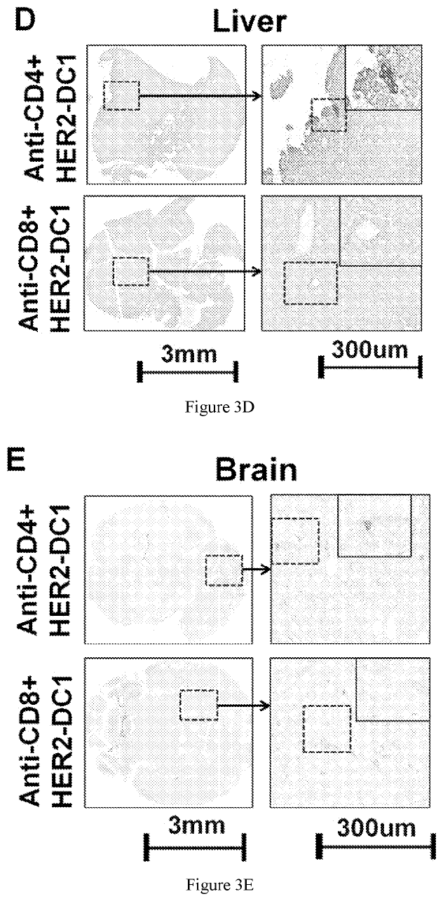

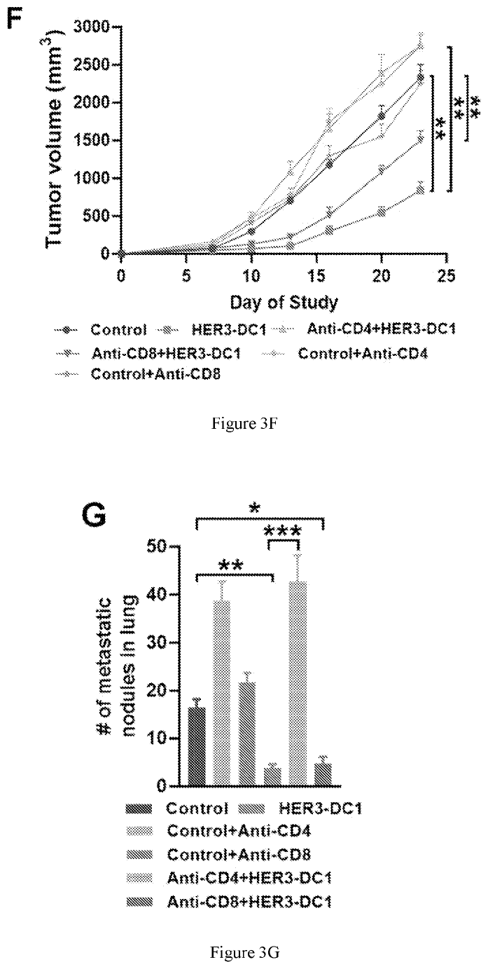

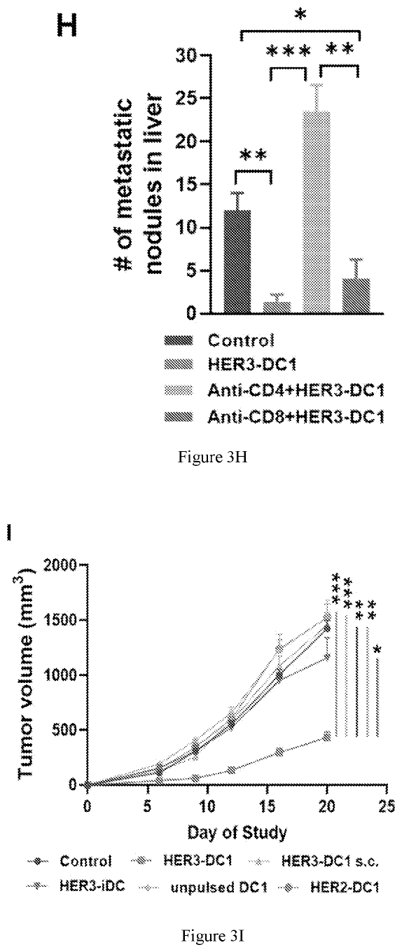

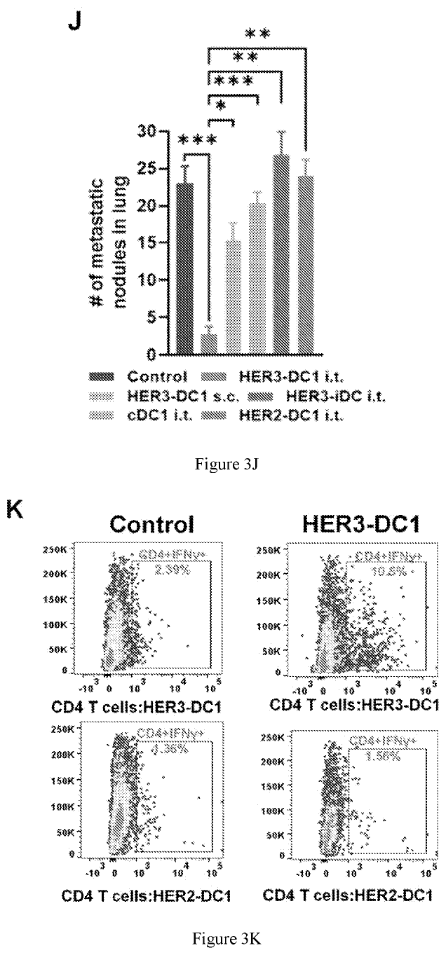

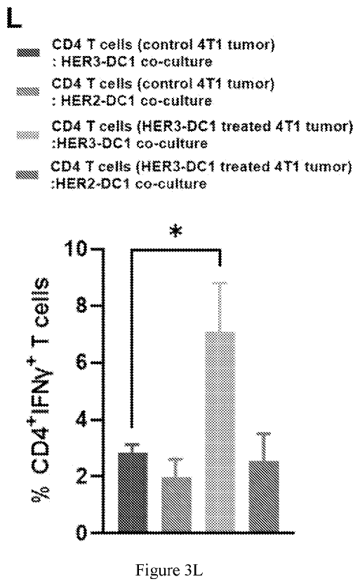

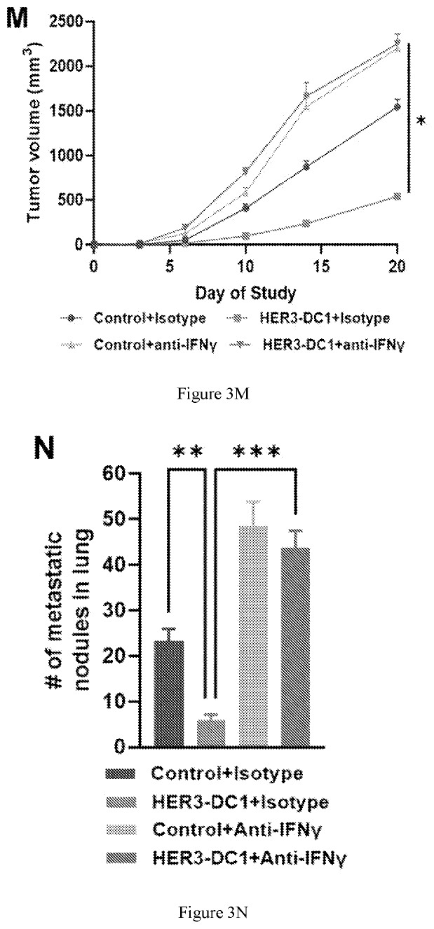

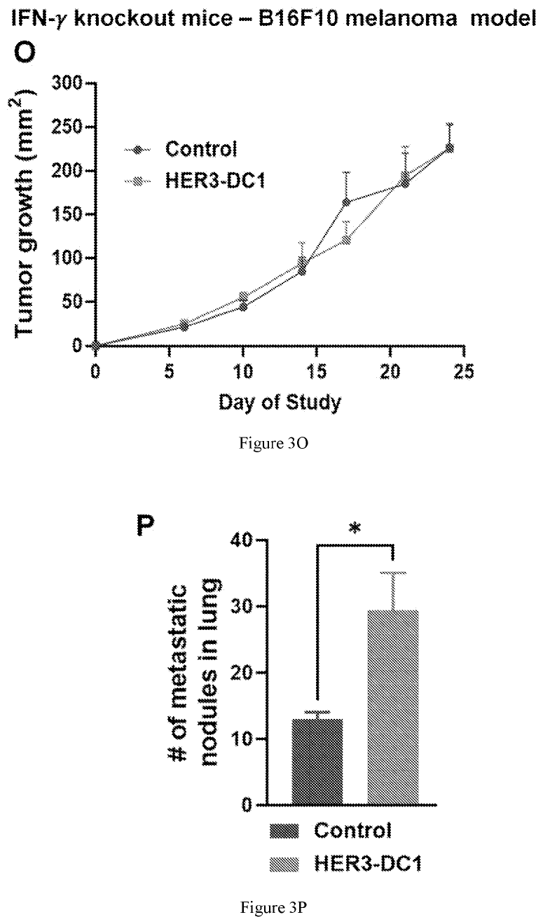

A critical role for tumor antigen specific CD4Th1 cells, was recently showed, in driving systemic and local anti-tumor immunity and eradicating tumors in HER2+ BC. To further understand the interplay of CD4Th1 cells in the tumor antigen pulsed cDC1 mediated anti-tumor response and metastasis prevention, a CD4 T cell depletion experiment was performed. As shown in FIGS. 3A and 3B, intramammary gland delivery of HER2-DC1 failed to control spontaneous mammary carcinoma growth and severity of disease progression in BALB-neuT mice in the absence of CD4+ T cells. These mice also developed spontaneous micro-metastasis in lung, liver and brain, suggesting the loss of metastasis preventive potential of HER2-DC1 when CD4 T cells are not present (FIGS. 3C-3E). To determine whether CD8 T cells have any impact on HER2-DC1 mediated anti-tumor response and metastasis preventive capacity, CD8 T cells were depleted in BALB-neuT mice. Interestingly, intramammary gland delivery of HER2-DC1 was able to control the tumor growth in mammary glands of BALB-neuT mice even in the absence of CD8 T cells (FIGS. 3A and 3B). The absence of micro-metastasis in lung, liver and brain, was also observed, after HER2-DC1 intramammary gland delivery in BALB-neuT mice with intact CD4 T cells and depletion of CD8 T cells (FIGS. 3C-3E).

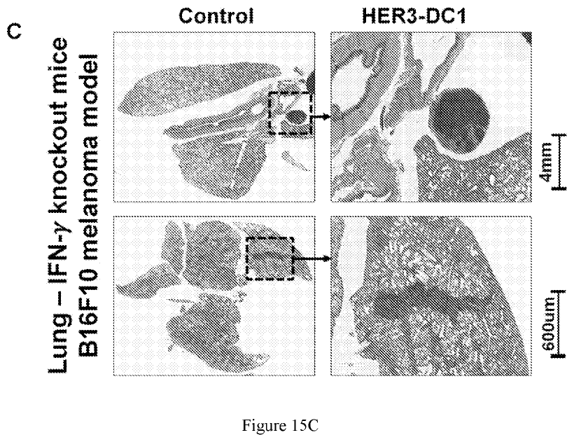

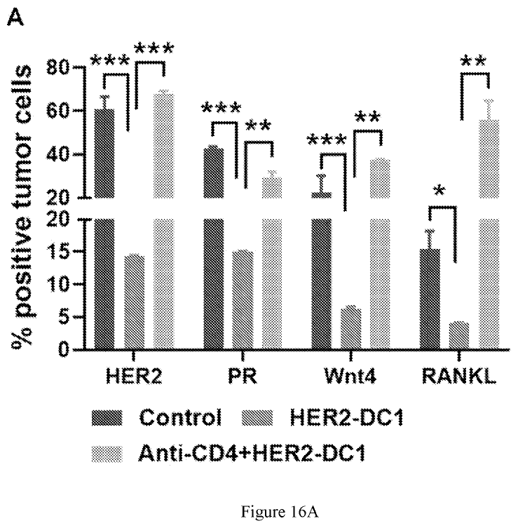

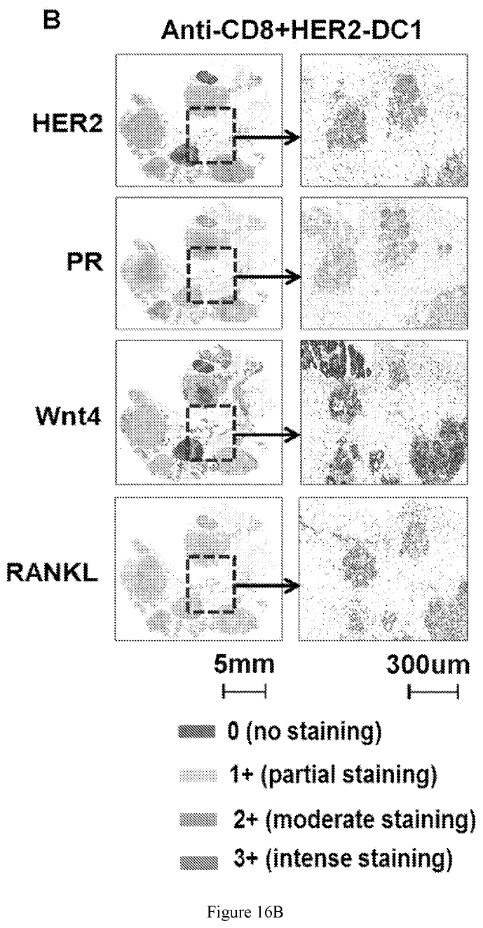

Similarly, absence of CD4 T cells, but not CD8 T cells, abrogated the primary tumor and metastasis inhibitory potential of HER3-DC1 intratumoral delivery in a 4T1 tumor model (FIGS. 3F-3H, 15A and 15B). Intratumoral delivery of tumor antigen targeting mature DC1 was critical for controlling primary tumor growth and inhibiting spontaneous metastasis (FIGS. 31 and 3J). This was supported by the failure of inhibiting primary tumors and metastasis in the 4T1 tumor model after subcutaneous delivery of HER3-DC1 (s.c.) and intratumoral delivery of immature HER3-iDC, unpulsed DC1 or HER2-DC1 (DC1 targeting irrelevant tumor antigen) (FIGS. 31 and 3J). Next, the specificity of the CD4Th1 response induced by intratumoral delivery of tumor antigen targeting DC1 was tested. Significantly increased CD4⁺IFN-γ⁺ T cells, were observed, after co-culturing CD4 T cells (from intratumoral HER3-DC1 treated 4T1 tumors) with HER3-DC1, compared to co-culture with HER2-DC1 or CD4 T cells from control 4T1 tumors co-cultured with HER3-DC1 or HER2-DC1 (FIGS. 3K and 3L). The critical contribution of the anti-tumor CD4Th1 response was further investigated using IFN-γ neutralizing antibody treatment in a 4T1 tumor model. Strikingly, the primary tumors and metastasis inhibitory capacity of HER3-DC1 intratumoral delivery was abrogated by IFN-γ neutralization in 4T1 tumor bearing mice (FIGS. 3M and 3N). Consistent with the TNBC spontaneous metastasis model, intratumoral delivery of HER3-DC1 failed to inhibit B16F10 primary melanoma growth and lung metastasis in IFN-γ knockout mice model (FIGS. 3O, 3P and 15C). Collectively, these results demonstrated that the anti-tumor CD4Th1 cells mediated immune response is necessary for prevention of overt metastasis.

### 4. Anti-Tumor CD4Th1 Immune Response Limits Cancer Cell Dissemination

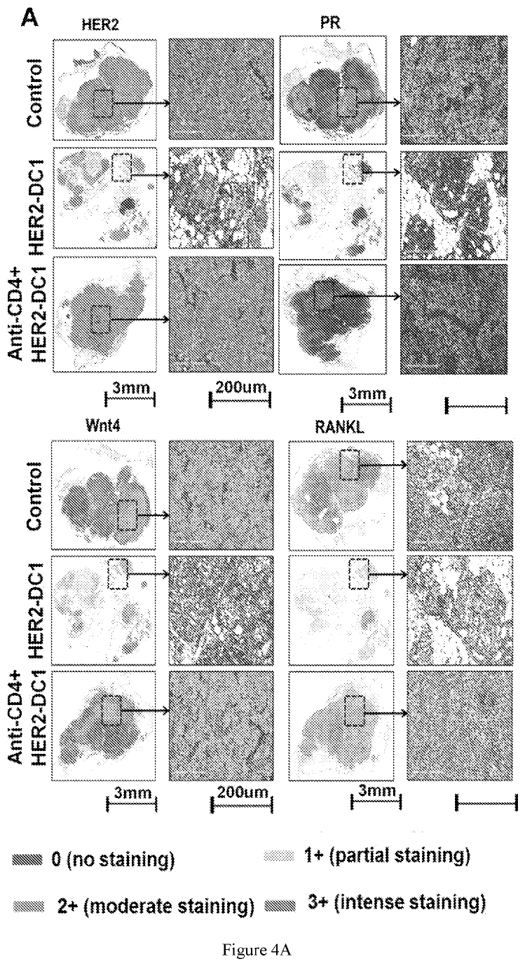

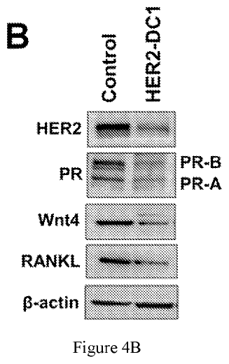

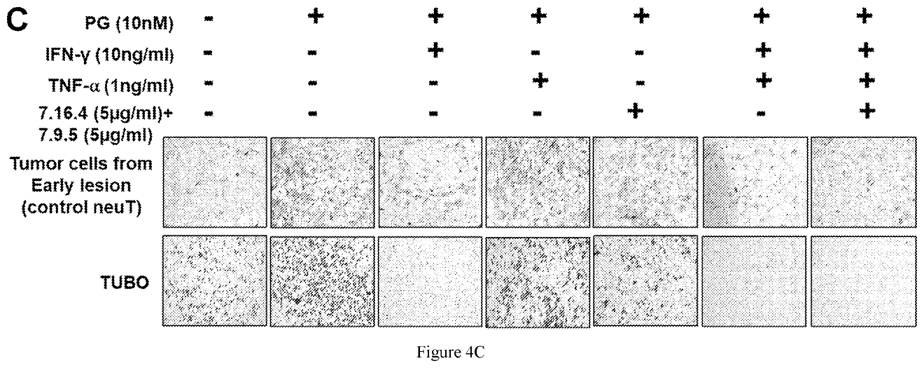

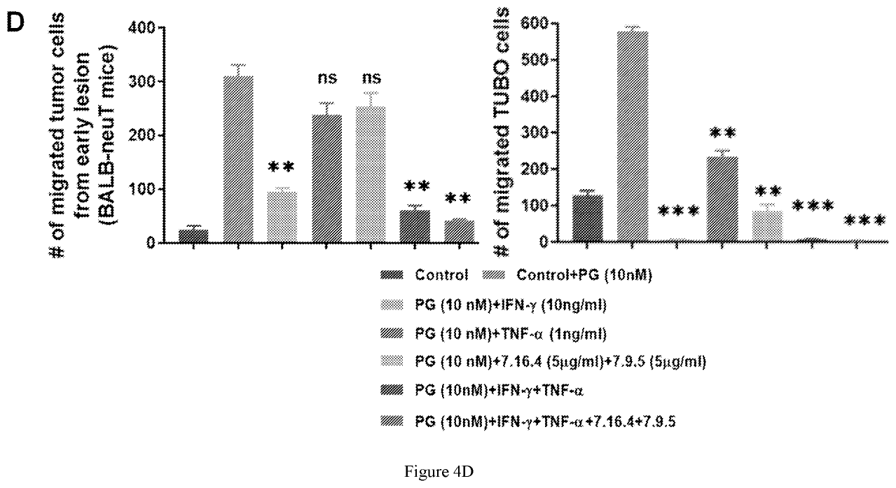

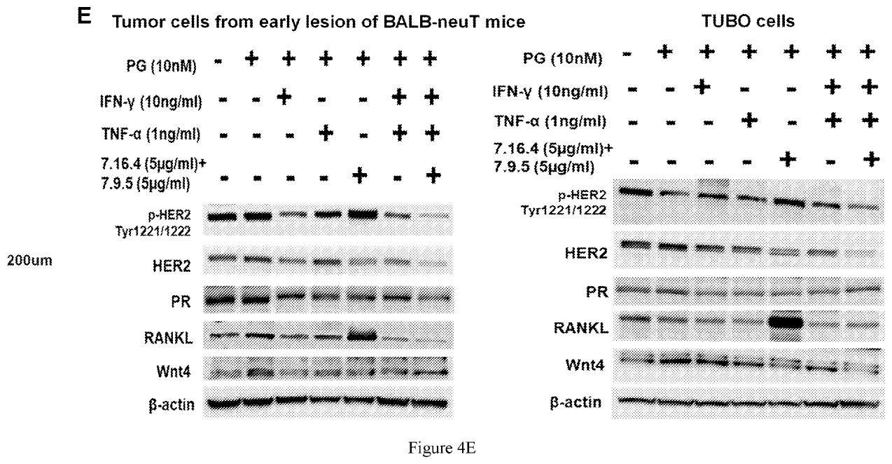

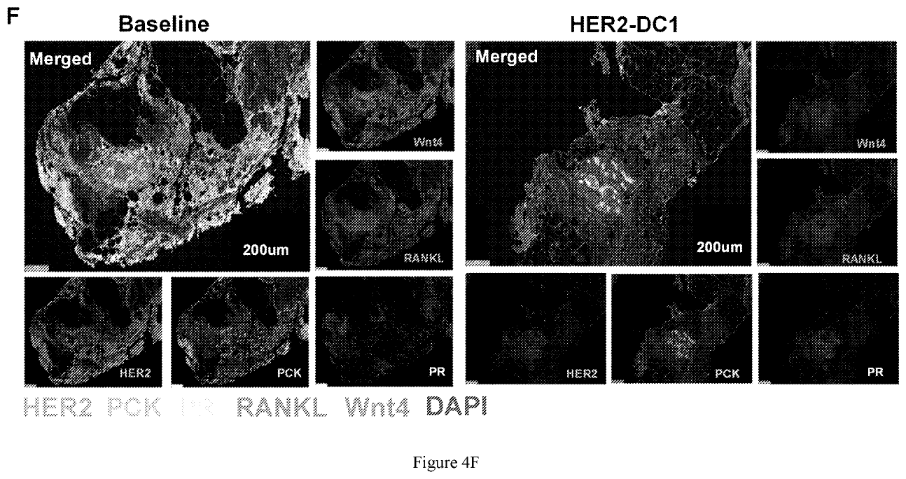

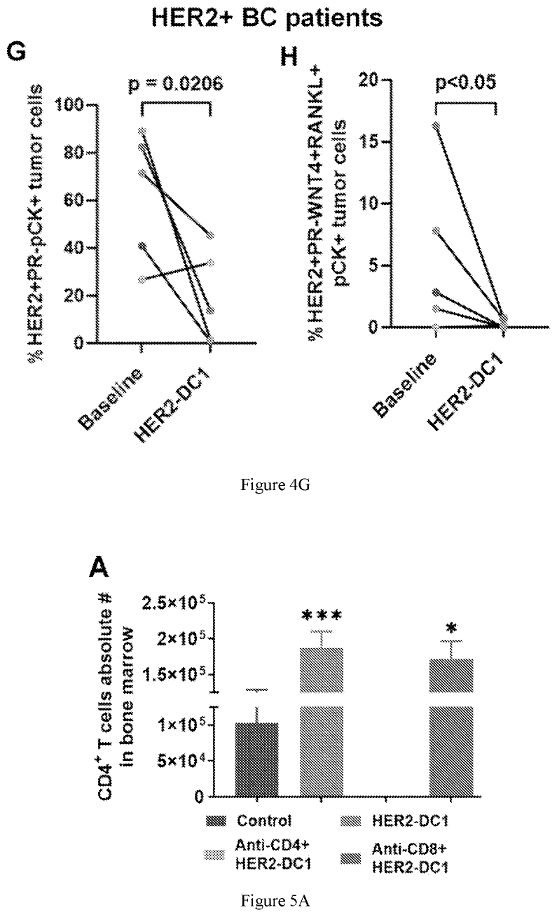

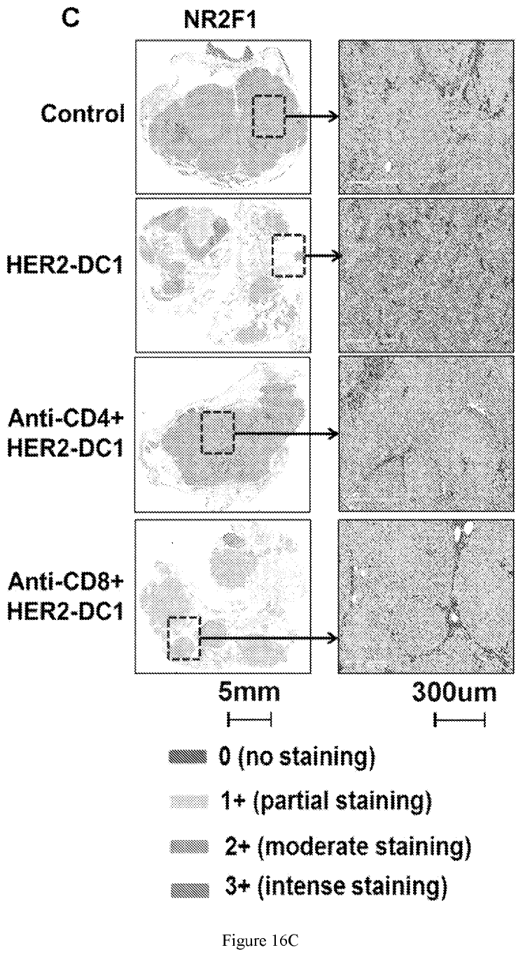

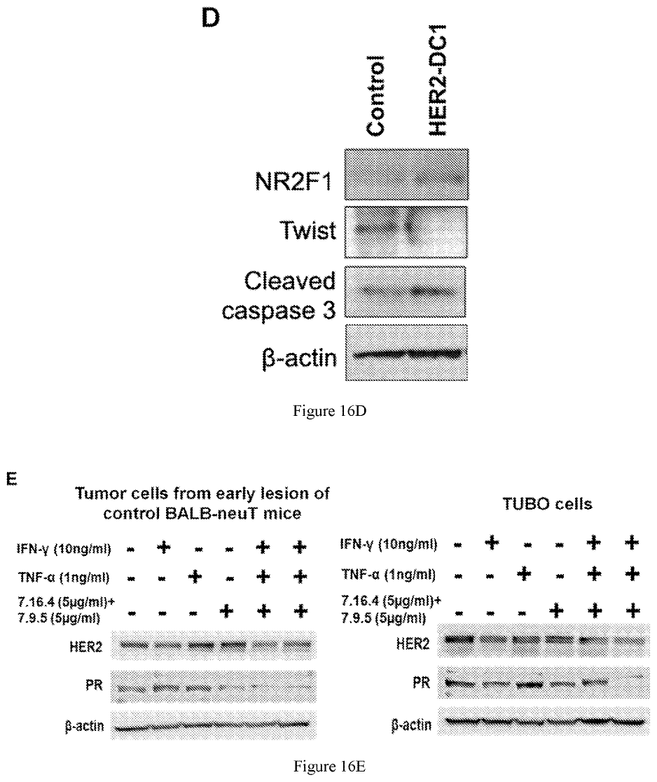

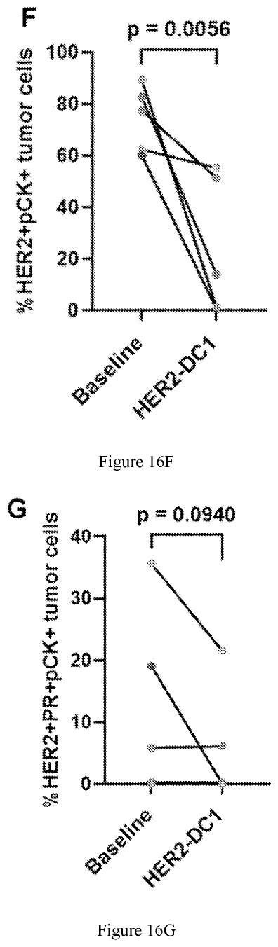

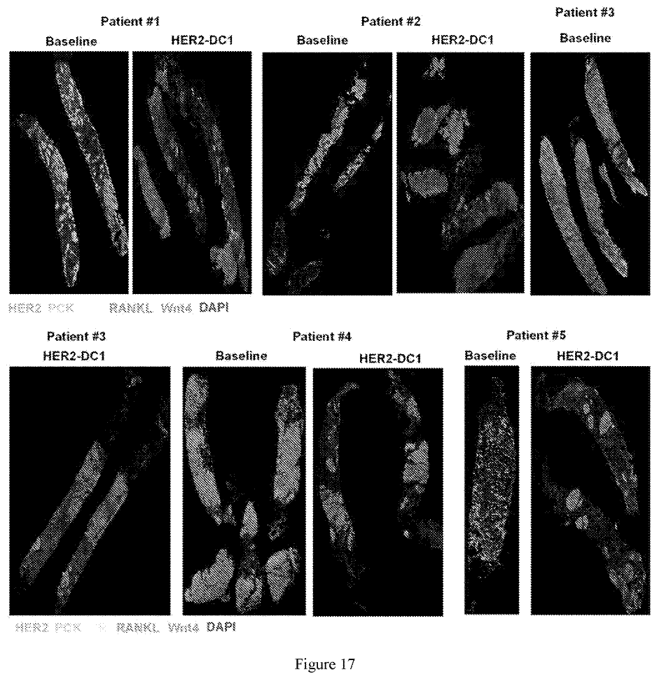

It was next sought to interrogate the efficacy of the intratumorally primed anti-tumor CD4Th1 immune response on BC cell dissemination by analyzing the status of dissemination mechanisms. Mammary glands positive for tumors of control and HER2-DC1 treated BALB-neuT mice at 16-18 weeks of age were collected. Consistent with prior work an increase in expression of HER2+ mammary carcinoma cell dissemination signaling proteins such as HER2, PR, Wnt4 and RANKL in tumor lesions from control BALB-neuT mice was confirmed (FIGS. 4A, 4B and 16A). As shown in FIGS. 4A, 4B and 16A, the increased expression of dissemination signaling proteins HER2, PR, Wnt4 and RANKL were markedly reduced in the tumor lesions of HER2-DC1 treated BALB-neuT mice. In addition, reduced basal level expression of NR2F1 and cleaved caspase 3, and increased expression of Twist in tumor lesions of control BALB-neuT mice were also found (FIGS. 16C and 16D). Conversely, tumor lesions of HER2-DC1 treated BALB-neuT mice had increased NR2F1 and cleaved caspase 3 expression and reduced Twist expression (FIGS. 16C and 16D). Interestingly, HER2-DC1 mediated modulatory effects on HER2+ mammary carcinoma cell dissemination signaling proteins, which were abolished in the absence of CD4+ T cells, but not by the depletion of CD8 T cells in BALB-neuT mice model (FIGS. 4A, 4B and 16A-16C). These data suggest that intratumoral priming of tumor antigen specific CD4Th1 cells alters cancer cell dissemination mechanisms in HER2 mammary carcinoma.

To further support the effects of the intratumorally primed anti-tumor CD4Th1 immune response in regulating HER2+ BC cell dissemination mechanisms, mammary tumor cells from control BALB-neuT mice or TUBO cells were stimulated with or without progesterone. Activation of progesterone receptor (PR) signaling with progesterone can promote HER2+ cancer cell migration from the early mammary lesion. Next, cells were treated with CD4Th1 cytokines IFN-γ, TNF-α, anti-HER2 antibodies (7.16.4 and 7.9.5) individually or in combination. Transwell invasion assays showed increased migration of tumor cells of control BALB-neuT mice mammary lesions and TUBO cells upon stimulation with progesterone compared to unstimulated control groups (FIGS. 4C and 4D). Importantly, IFN-γ treatment significantly reduced progesterone-induced migration of tumor cells from BALB-neuT mice mammary lesions and TUBO cells (FIGS. 4C and 4D). TNF-α and anti-HER2 antibodies treatment showed only modest effects on reducing progesterone induced migration of control BALB-neuT mammary tumor cells, while these treatment effects were significant in TUBO cells (FIGS. 4C and 4D). These results suggest that IFN-γ treatment alone exhibited maximal inhibitory effects against progesterone induced migration of HER2+ mammary carcinoma cells. As shown in FIGS. 4E and 16E, IFN-γ treatment alone reduced protein expression and autophosphorylation of HER2 and PR in tumor cells from BALB-neuT mice mammary lesions and TUBO cells, with or without progesterone stimulation. When combined with TNF-α or anti-HER2 antibodies, expression of these proteins was further reduced. Next, stimulation of tumor cells from BALB-neuT mice mammary lesions and TUBO cells with progesterone led to an increased expression of the HER2+ BC dissemination proteins Wnt4 and RANKL (FIG. 4E). Interestingly, IFN-γ alone or in combination with TNF-α reduced progesterone signaling mediated expression of Wnt4 and RANKL in these tumor cells (FIG. 4E). It was also found that combination treatment of IFN-γ, TNF-α and anti-HER2 antibodies further downregulated progesterone-mediated Wnt4 and RANKL expression (FIG. 4E). These data further support the finding that CD4Th1 response limits HER2+ BC cell dissemination by altering dissemination mechanisms.

To further provide clinical evidence for the inhibitory potential of the anti-tumor CDTh1 response on BC cell dissemination, quantitative multiplex immunofluorescence (mIF) staining was performed on baseline and intratumoral HER2-DC1 treated tumor biopsies of HER2+ BC patients. A marked reduction in the levels of HER2+pCK+ and HER2+PR+pCK+ tumor cells was observed after intratumoral HER2-DC1 delivery in HER2+ BC patients compared to their baseline tumor biopsy (FIGS. 4F, 16F, 16G and 17). Remarkably, intratumoral HER2-DC1 delivery significantly inhibited disseminating cancer cell phenotypes HER2+PR-pCK+ and HER2+PR-WNT4+RANKL+pCK+ in HER2+ BC patients (FIGS. 4F-4H and 17). These data provide clinical evidence that intratumoral priming of anti-tumor CD4Th1 cells limits BC cell dissemination from the primary tumor lesion in patients.

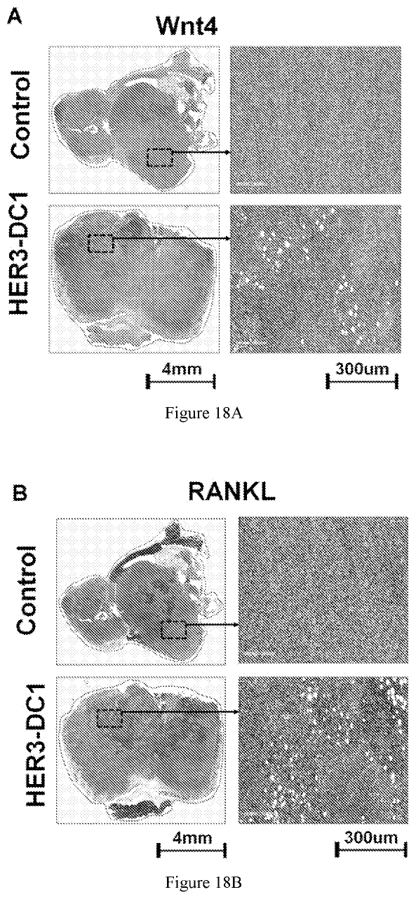

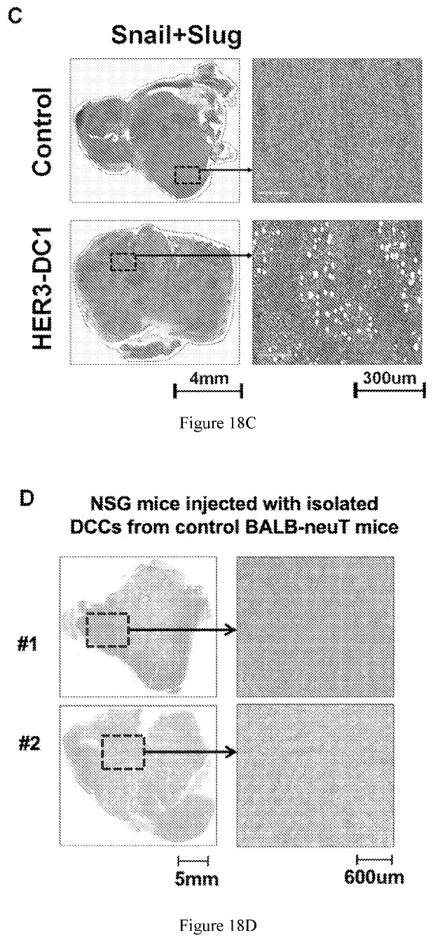

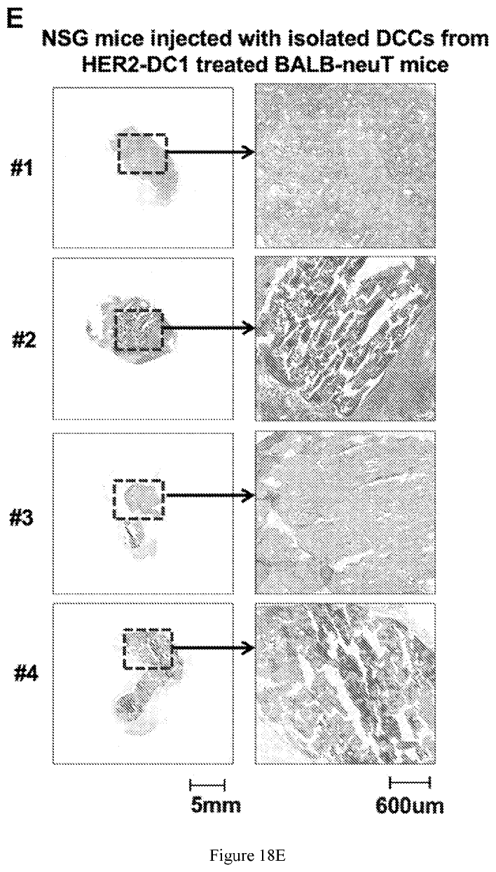

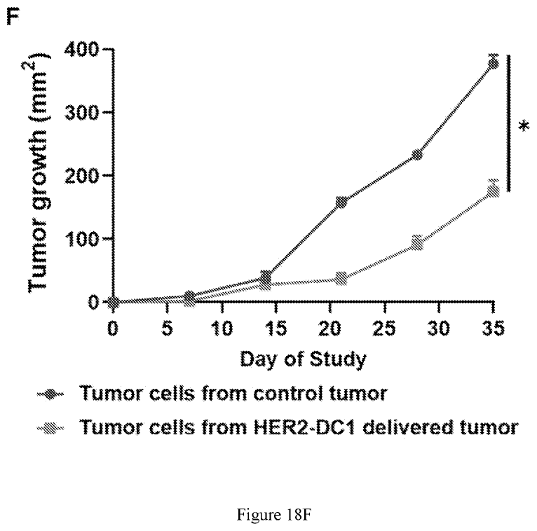

Similar to the BALB-neuT spontaneous mammary carcinoma tumor model and HER2+BC patients, the expression status of cancer cell dissemination mechanisms in a 4T1 tumor model was also assessed. Orthotopically transplanted 4T1 tumors of control and HER3-DC1 treated mice were collected and analyzed for Wnt4, RANKL, Snail and Slug proteins. The control 4T1 tumors expressed increased cancer cell dissemination proteins Wnt4, RANKL, Snail and Slug (FIGS. 18A-18C). Importantly, intratumoral delivery of HER3-DC1 decreased expression of Wnt4, RANKL, Snail and Slug in the 4T1 tumors (FIGS. 18A-18C), indicating 20 that the anti-HER3 CD4Th1 immune response targets dissemination signaling and prevent 4T1 tumor cell dissemination and overt metastasis outgrowth in distant organs.

### 5. Anti-Tumor CD4Th1 Immune Response Targets DCCs

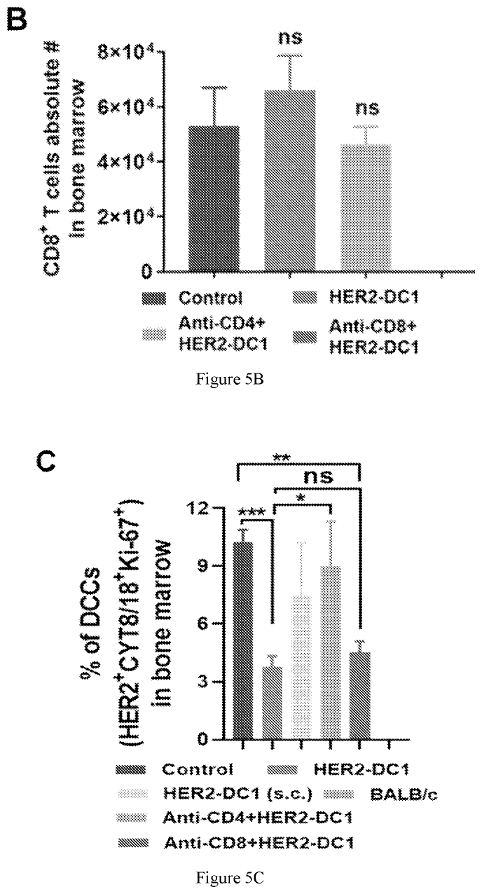

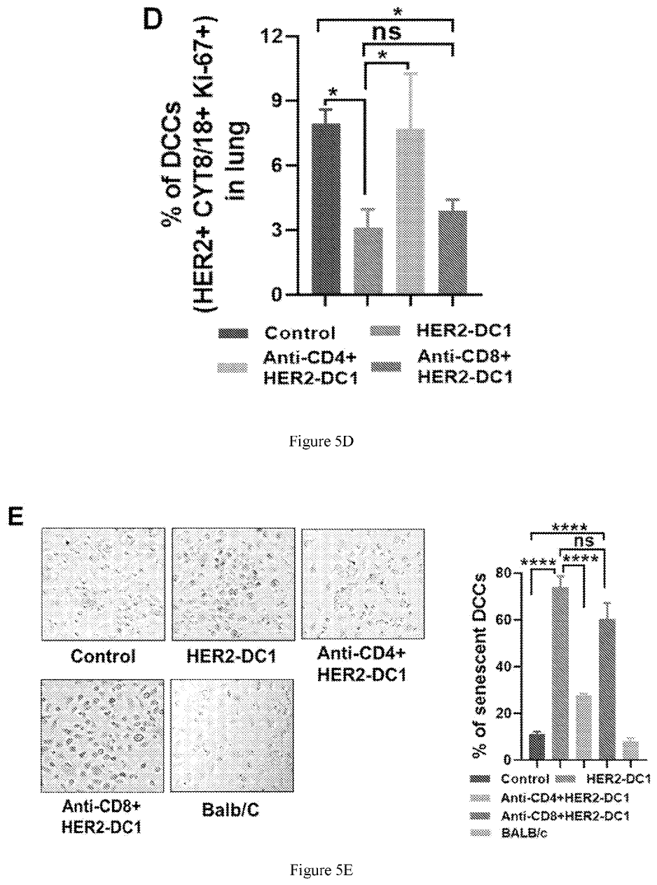

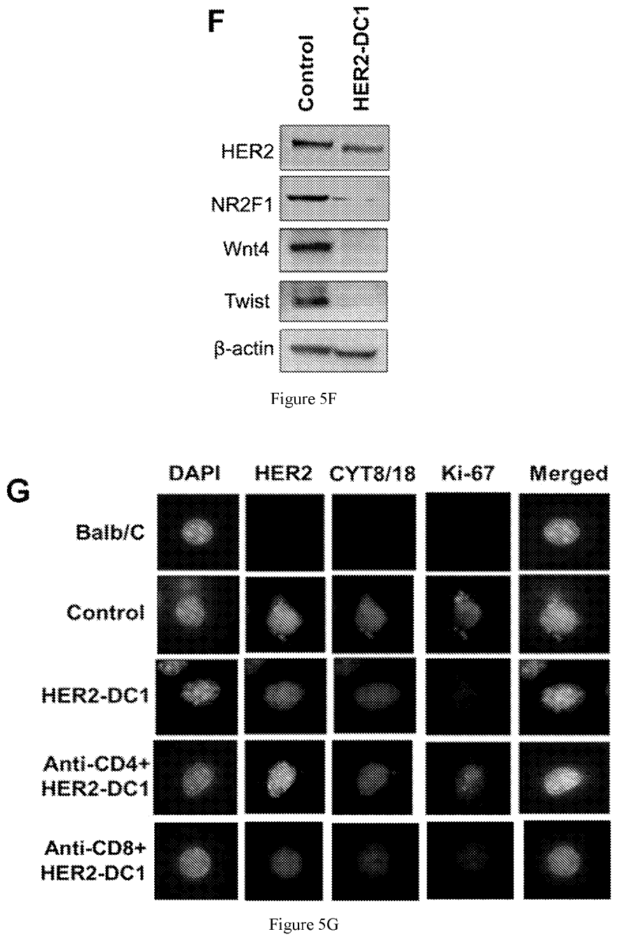

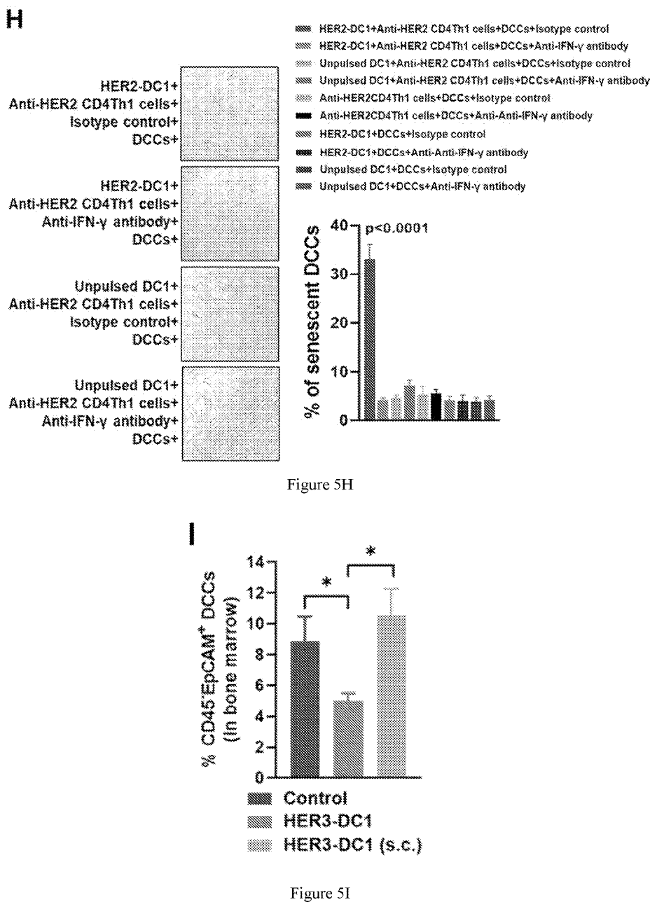

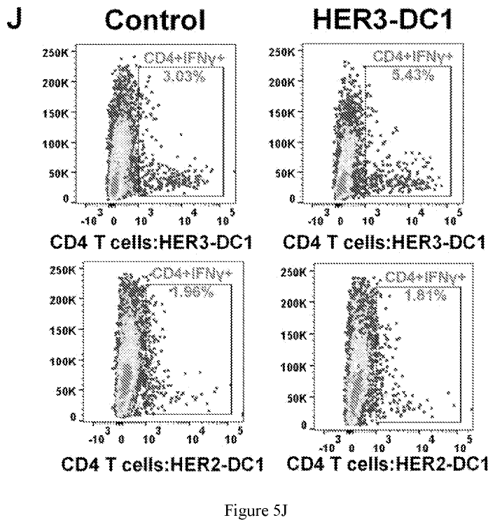

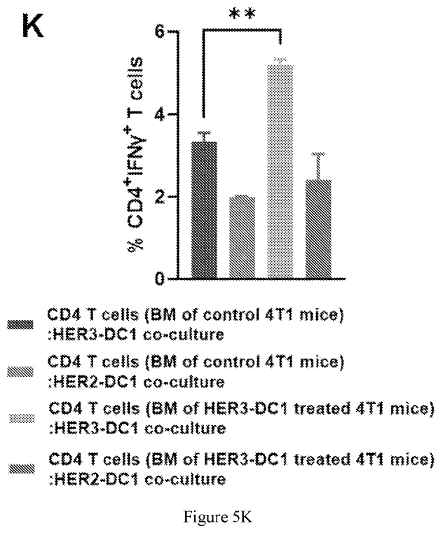

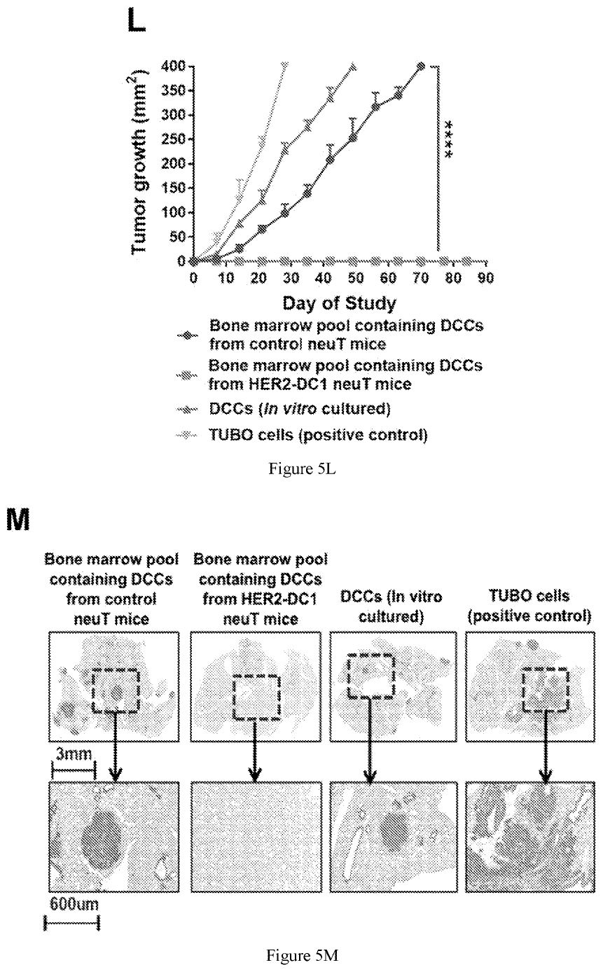

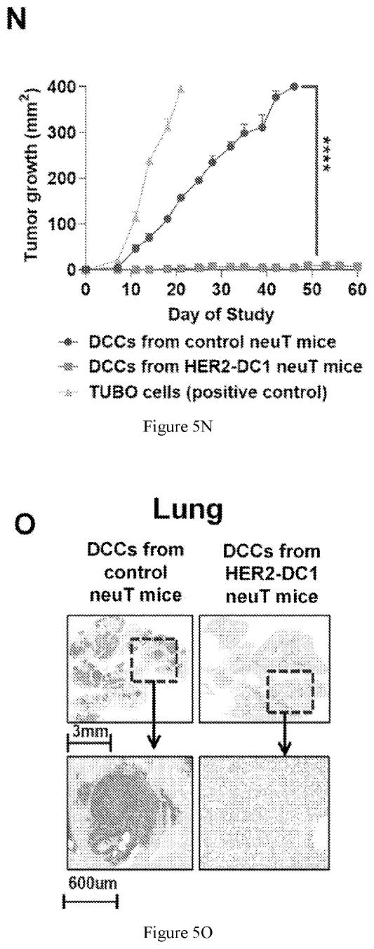

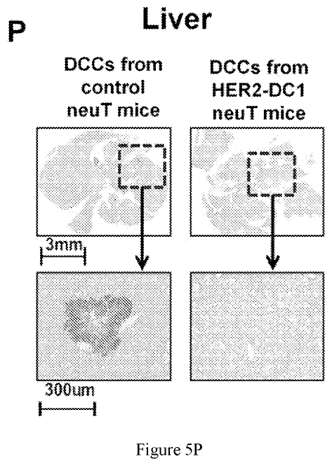

The DCCs present in the BM of BALB-neuT mice with early lesion or primary mammary carcinoma have been reported to migrate early and develop overt metastases. Next investigated was the efficacy of the HER2-DC1 primed CD4Th1 immune response on DCCs in BALB-neuT mice model. BM from control or HER2-DC1 treated BALB-neuT mice were first analyzed for CD4 T cells and CD8 T cells infiltration at 16-18 weeks of age by flow cytometry. As shown in FIGS. 5A and 5B, a significant increase in the level of CD4 T cells with modest changes in CD8 T cells infiltration was observed in the BM of HER2-DC1 treated BALB-neuT mice compared to control. Interestingly, intramammary gland HER2-DC1 treated BALB-neuT mice had reduced levels of DCCs in their BM and lungs compared to control BALB-neuT mice (FIGS. 5C and 5D). No impact on the level of DCCs in the BM of subcutaneous HER2-DC1 treated BALB-neuT mice compared to intramammary gland HER2-DC1 treatment was noted (FIG. 5C). These DCCs also remained at reduced levels in the mice depleted of CD8 T cells, however this effect was abrogated in the absence of CD4 T cells in BALB-neuT mice after HER2-DC1 delivery (FIGS. 5C and 5D). Also observed were significantly increased senescent DCCs in the BM of HER2-DC1 treated BALB-neuT mice compared to control, based on SA-β-gal staining (FIG. 5E). This increased senescent DCCs level was reversed only in the absence of CD4 T cells but not by the depletion of CD8 T cells in BALB-neuT mice following HER2-DC1 delivery (FIG. 5E).

Western blot analysis revealed reduced protein expression of HER2, stemness/dormancy marker NR2F1, and EMT markers Wnt4 and Twist in DCCs from HER2-DC1 treated BALB-neuT mice compared to control mice (FIG. 5F). Immunofluorescence staining also demonstrated that HER2 and Ki-67 expression was reduced in DCCs from the BM of HER2-DC1 treated BALB-neuT mice (FIG. 5G). However, these changes were not apparent in the absence of CD4+ T cells (FIG. 5G).

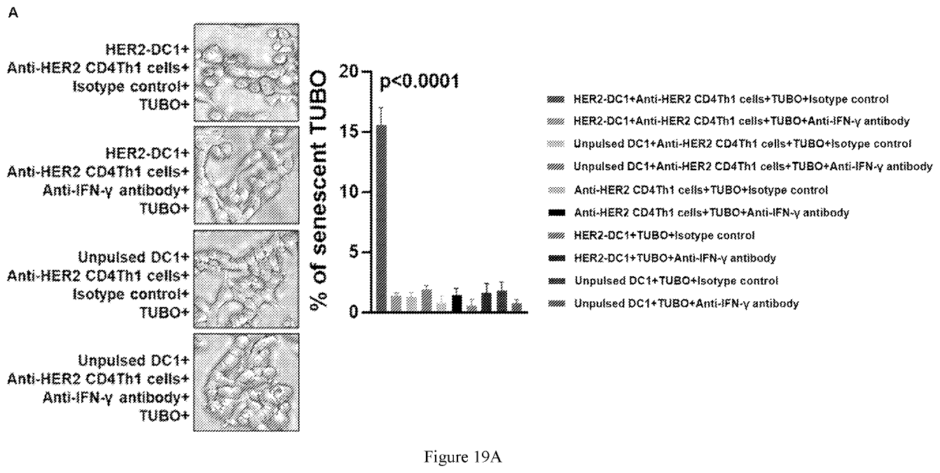

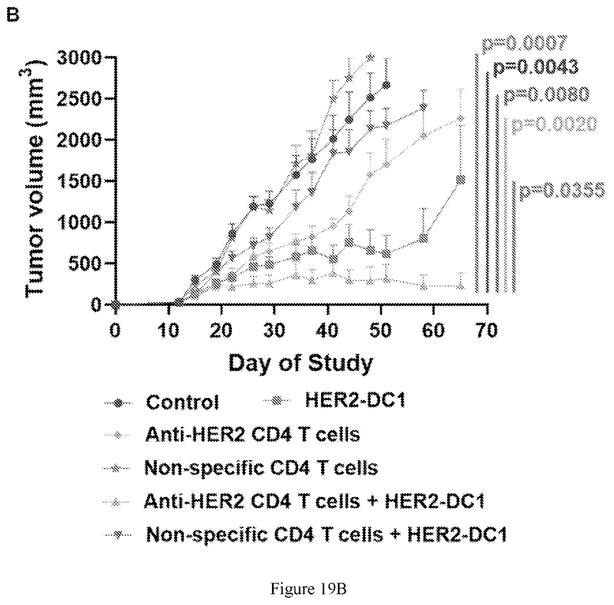

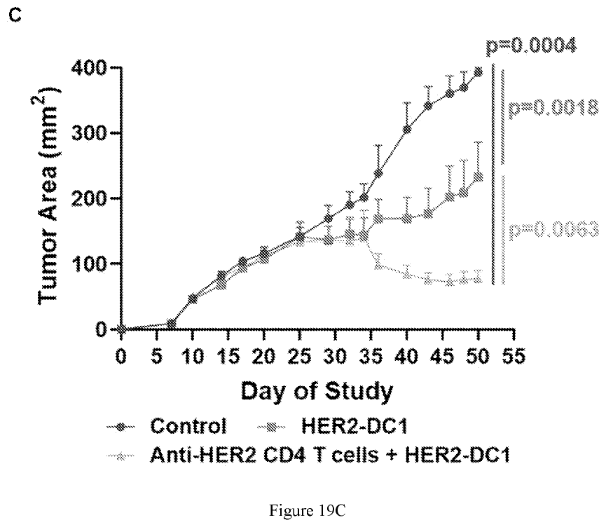

Next, investigated the specificity and functional activity of anti-tumor CD4Th1 cells on DCCs using a transwell migration assay were investigated. Activation and priming of anti-HER2 CD4Th1 cells (from HER2-DC1 treated BALB-neuT mice) with HER2-DC1 was able to significantly increase the level of senescent DCCs (from control BALB-neuT mice) compared to anti-HER2 CD4Th1 cells activated with unpulsed DC1 (FIG. 5H). The contribution of anti-HER2 CD4Th1 cells secreting IFN-γ in inducing senescence in DCCs, was confirmed, by IFN-γ neutralization in the co-culture (FIG. 5H). These observations were also consistent in TUBO cells for anti-HER2 CD4Th1 cells and HER2-DC1 co-culture with or without IFN-γ neutralization (FIG. 19A). To further demonstrate the functional role of tumor antigen specific CD4Th1 cells, adoptive transfer of anti-HER2 CD4Th1 cells or non-specific CD4 T cells with HER2-DC1 was performed in BALB/c mice bearing orthotopic HER2+ TUBO tumors. The intratumoral delivery of anti-HER2 CD4Th1 cells combined with HER2-DC1 induced a superior antitumor response with complete tumor regression in 50% of treated mice compared to intratumoral delivery of non-specific CD4 T cells (from naïve BALB/c mice) combined with HER2-DC1 or monotherapy groups (FIG. 19B). Similarly, adoptive transfer of anti-HER2 CD4Th1 cells (intravenous delivery) combined with intratumoral HER2-DC1 delivery significantly inhibited the tumor growth in BALB/c mice bearing subcutaneous HER2+ TUBO tumors (FIG. 19C).

The study was extended to a 4T1 spontaneous metastasis model and observed that intratumoral delivery of HER3-DC1 reduced the level of DCCs in the BM of 4T1 tumor bearing mice when compared to control and subcutaneous HER3-DC1 delivery groups (FIG. 5I). Next, the specificity and functional status of CD4Th1 cells from the BM of control and intratumoral HER3-DC1 treated 4T1 tumor bearing mice was examined. A significant increase in the level of CD4⁺IFN-γ⁺ T cells was observed after co-culturing CD4 T cells (isolated from the BM of HER3-DC1 treated 4T1 mice) with HER3-DC1 (FIGS. 5J and 5K). However, no change was observed when these CD4 T cells were co-cultured with HER2-DC1 (FIGS. 5J and 5K), highlighting the priming and propagation potential of highly tumor antigen specific anti-tumor CD4Th1 cells in the BM to target DCCs in TNBC.

To evaluate the tumorigenic and metastatic seeding potential of DCCs in the BM of control and HER2-DC1 treated BALB-neuT mice, NSG mice experiments were performed. Subcutaneous injection of total BM pool containing DCCs from untreated control BALB-neuT mice into NSG mice resulted in the development of primary tumors and pulmonary metastases (FIGS. 5L and 5M). In contrast, no palpable tumors and overt lung metastases were formed in NSG mice (up to 120 days) following injection of total BM pool containing DCCs from HER2-DC1 treated BALB-neuT mice (FIGS. 5L and 5M). Since the intratumoral priming of anti-tumor CD4Th1 cells reduced the number of DCCs in the BM, an equal number of isolated BM DCCs from the experimental BALB-neuT mice were injected into NSG mice to provide more specific evidence for the inhibition of DCC tumorigenesis by the CD4Th1 immune response. Primary tumor growth and metastasis in lung and liver was observed in NSG mice following injection of purified and same number of DCCs from the untreated control BALB-neuT mice (FIGS. 5N-5P and 18D). Strikingly, injection of purified and an identical number of DCCs from the BM of HER2-DC1 treated BALB-neuT mice into NSG mice did not result in the development of primary tumors and micro-metastases in distant organs, lung and liver (up to 120 days) (FIGS. 5N-5P and 18E). Next assessed was the impact of active metastatic tumor cells isolated from the subcutaneous HER2+ tumors of control and HER2-DC1 treated BALB/c mice. Although a significant delay was observed in tumor growth in NSG mice that were subcutaneously transplanted with metastatic tumor cells of HER2-DC1 treated mice compared to control tumors, the HER2-DC1 treated metastatic tumor cells still formed active metastasis (FIG. 18F). Collectively, these results provide evidence that the tumor antigen specific CD4Th1 immune response targets more specifically DCCs in distant organs and inhibits their tumorigenic and metastasis growth potential in breast carcinogenesis.

### 6. Anti-Tumor CD4Th1 Response Render Immune Recognition of DCCs

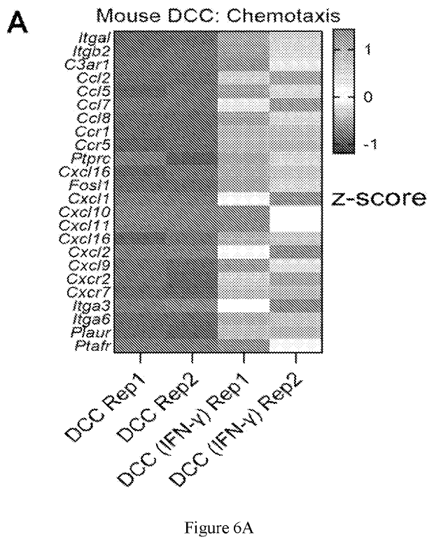

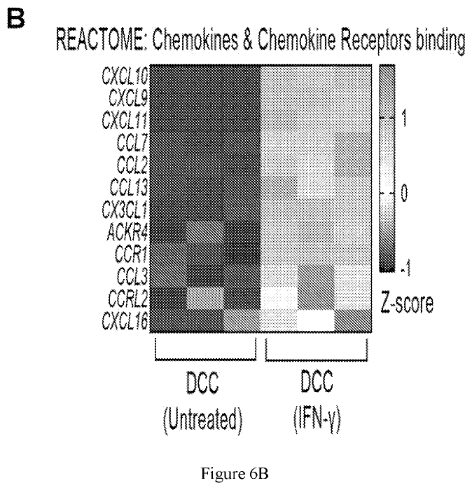

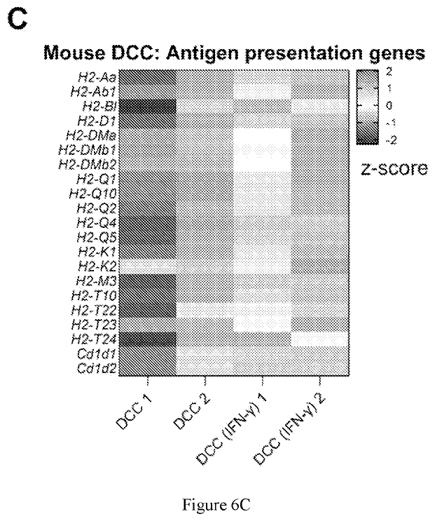

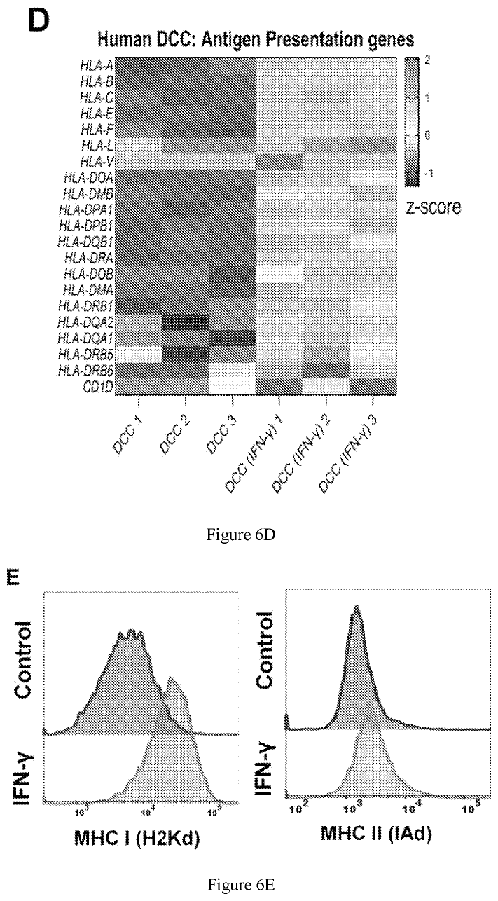

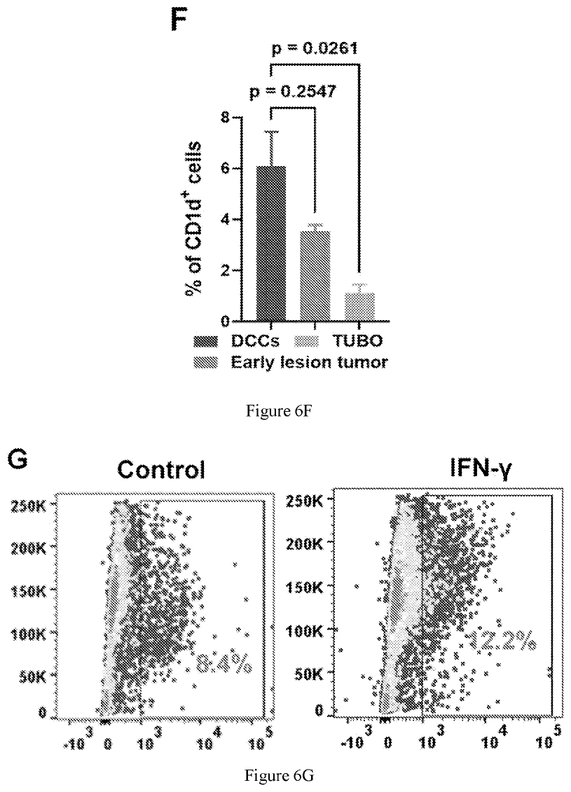

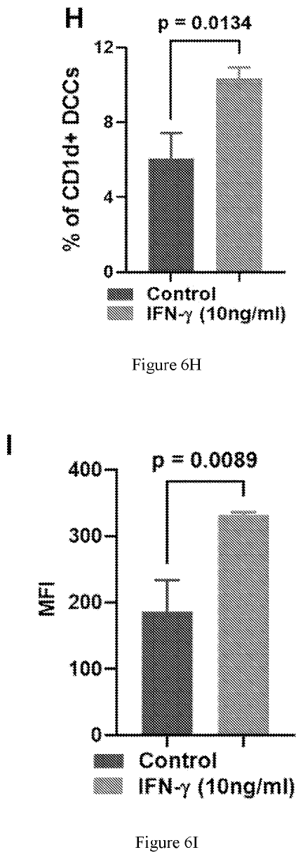

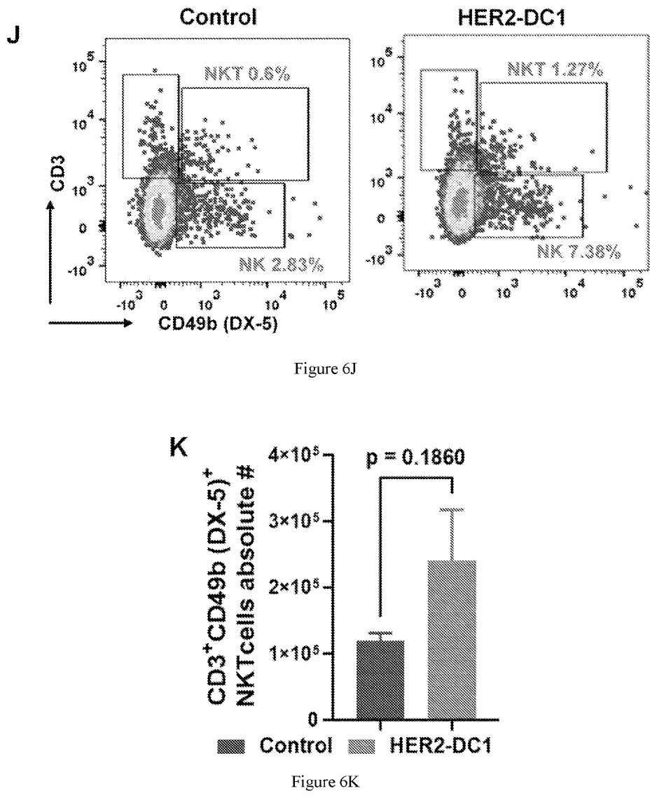

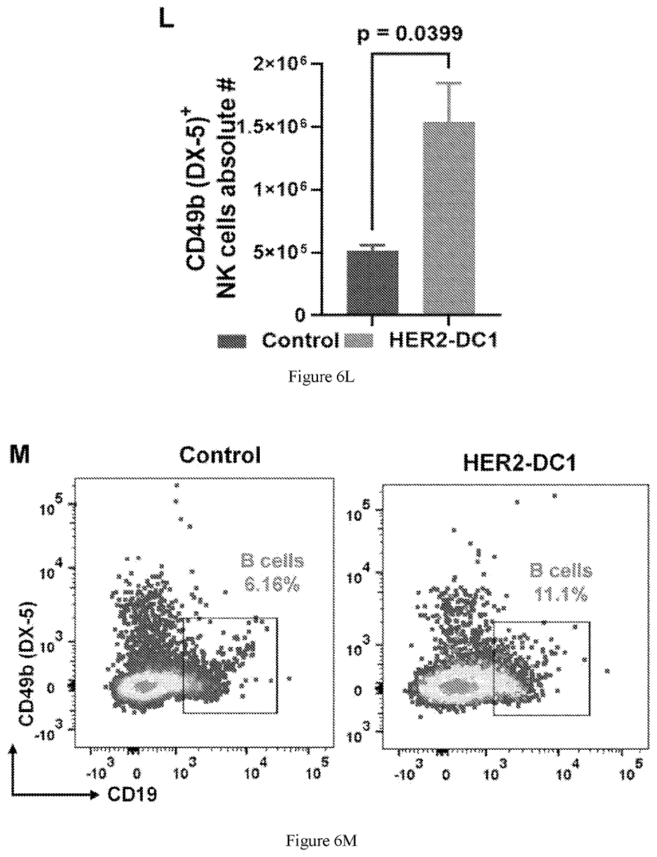

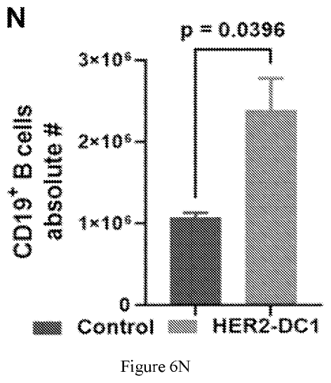

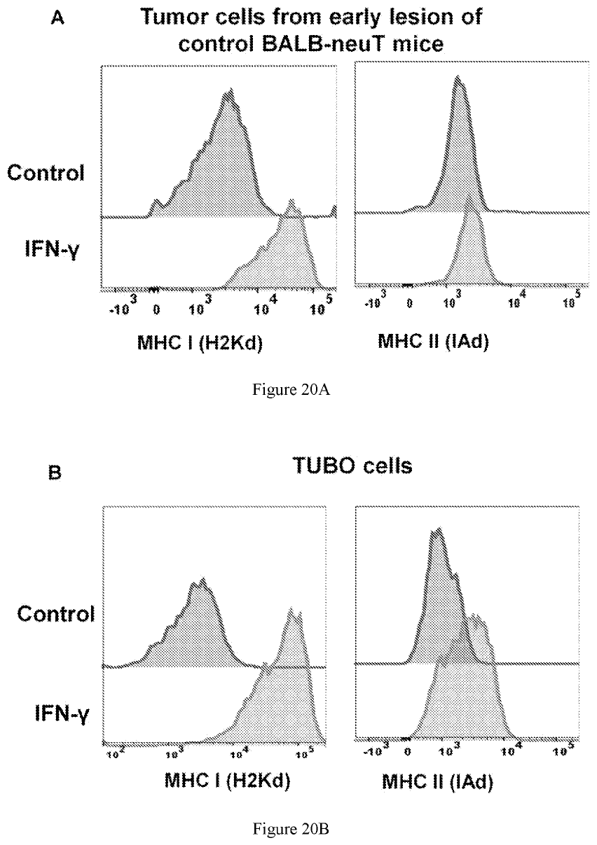

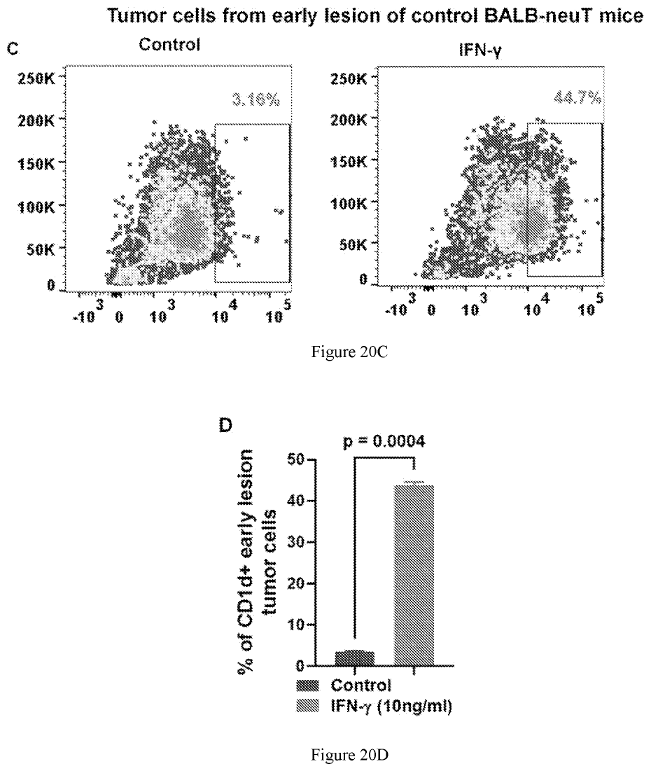

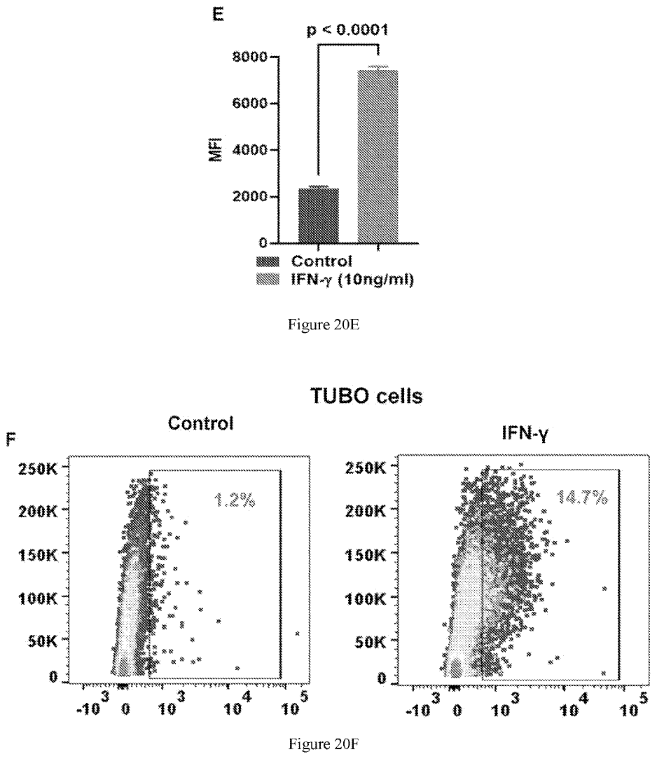

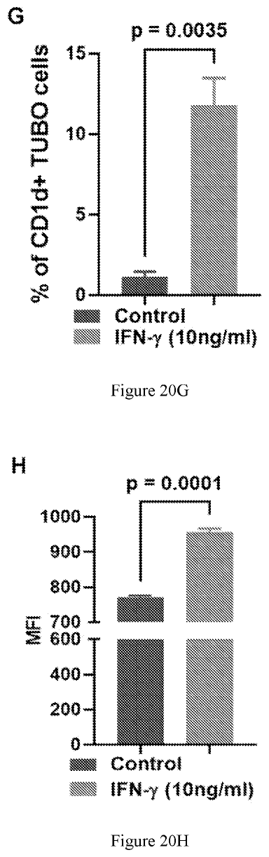

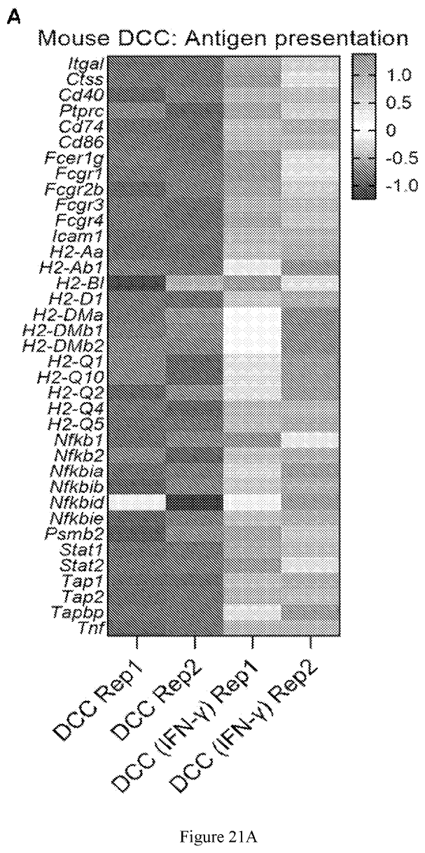

Differential gene expression analysis from the RNA sequencing experiments showed downregulation of various chemokine genes, MHC class I and MHC class II gene subsets, and CD1d genes in DCCs of BALB-neuT mice and HER2+ BC patients (FIGS. 6A-6D). It was further confirmed decreased cell surface expression of MHC class I, MHC class II and CD1d in DCCs of BALB-neuT mice (FIGS. 6E-6G). This observation was consistent in tumor cells from early mammary lesions of BALB-neuT mice and TUBO cells (FIGS. 20A-20H).

Together these data suggest that DCCs become invisible to CD4 T, CD8 T, B and NKT cells and escape immunosurveillance. Observed next was that CD4Th1 cytokine IFN-γ upregulated the expression of CXCL9, CXCL10, CXCL11, CCL7 and CXCL16 chemokine gene signatures in DCCs of BALB-neuT mice (FIG. 6A). Strikingly, IFN-γ treatment also upregulated the expression of the chemokine genes CXCL9, CXCL10, CXCL11, CCL7, CXCL16 and CX3CL1 in DCCs of HER2+ BC patients (FIGS. 6B and 21B). These chemokines are involved in the chemoattraction of CD4 T, CD8 T, B, NK, NKT cells and cDC1 and drive strong anti-tumor immunity. Notably, MHC class I and class II gene subsets and antigen presentation machinery were upregulated in DCCs of BALB-neuT mice and HER2+ BC patients following IFN-γ treatment, compared to untreated control DCCs (FIGS. 6C, 6D, 21A, and 21C-21F). Further confirmed was an increase in cell surface expression of MHC class I, MHC class II and CD1d following IFN-γ treatment in DCCs of BALB-neuT mice (FIGS. 6E and 6G-6I). Similarly, IFN-γ treatment also increased cell surface expression of MHC class I, MHC class II and CD1d in tumor cells of BALB-neuT mice mammary lesions and TUBO cells (FIGS. 20A-20H). The CD1d+ population was higher in DCCs of BALB-neuT mice compared to early mammary lesion and TUBO cells (FIG. 6F). Interestingly, IFN-γ treatment increased the percentage of the CD1d+ population in all three cell types (FIGS. 6G, 6H, 20C, 20D, 20F, and 20G), highlighting a novel role for CD4Th1 response in regulating CD1d expression in tumor cells.

After observing the increased infiltration of CD4 and CD8 T cells (FIGS. 5A and 5B), it was then tested if intratumoral priming of anti-tumor CD4Th1 cells can also drive NKT, NK and B cells into the BM to target DCCs in a BALB-neuT mice model. Consistent with the possibility of in vitro findings, intratumoral priming of anti-tumor CD4Th1 cells increased infiltration of NKT, NK and B cells in the BM of BALB-neuT mice compared to untreated control BALB-neuT mice (FIGS. 6J-6N). Collectively, these data suggested that the intratumoral delivery of tumor antigen targeting cDC1 drive anti-tumor CD4Th1 responses as well as infiltration of NKT, NK and B cells in the BM. These immune effector cells may collectively enhance chemokines, MHC class I, MHC class II, and CD1d expression in DCCs, leading to increased immune recognition, antigen presentation, and the facilitation of strong anti-tumor immunity to eradicate DCCs in distant organs in BC.

### 7. CD4Th1 Cytokine IFN-γ Inhibits Tumorigenesis of DCCs

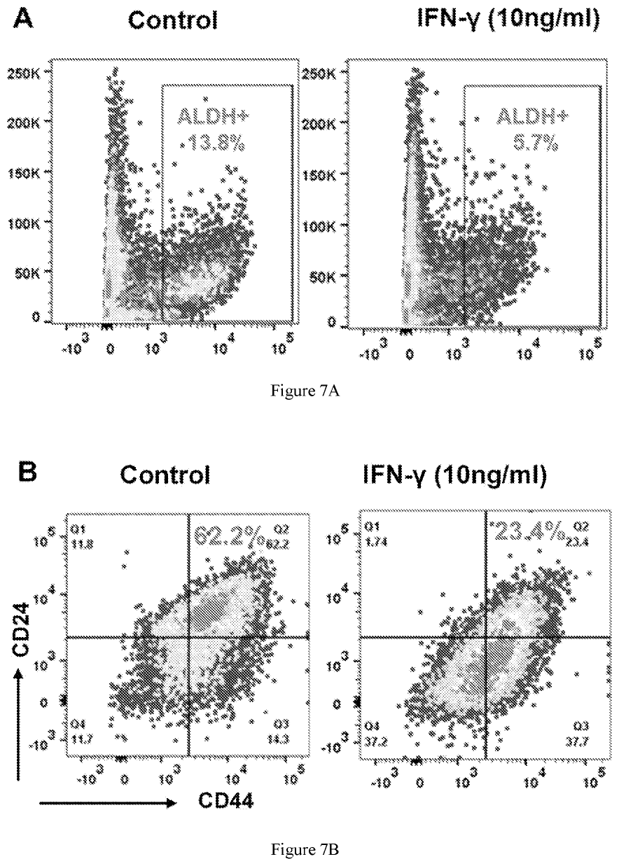

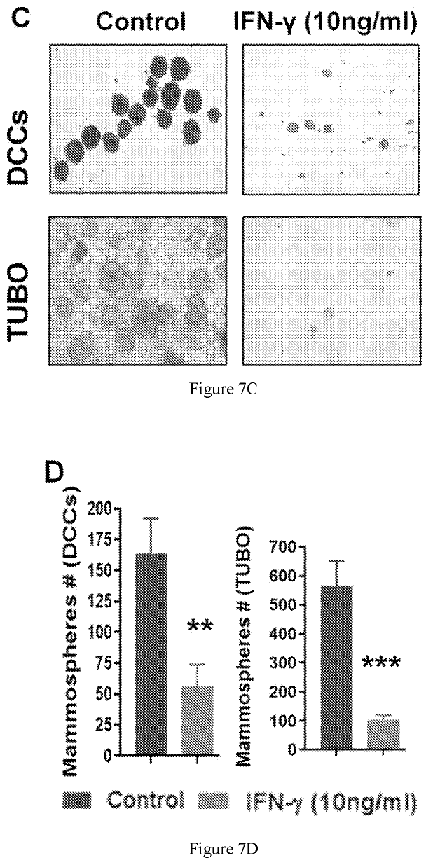

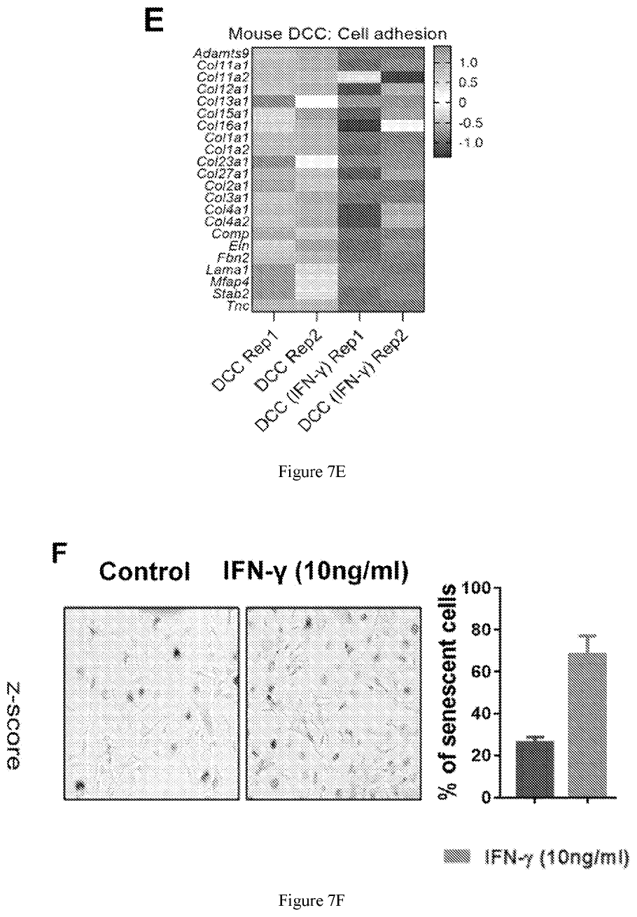

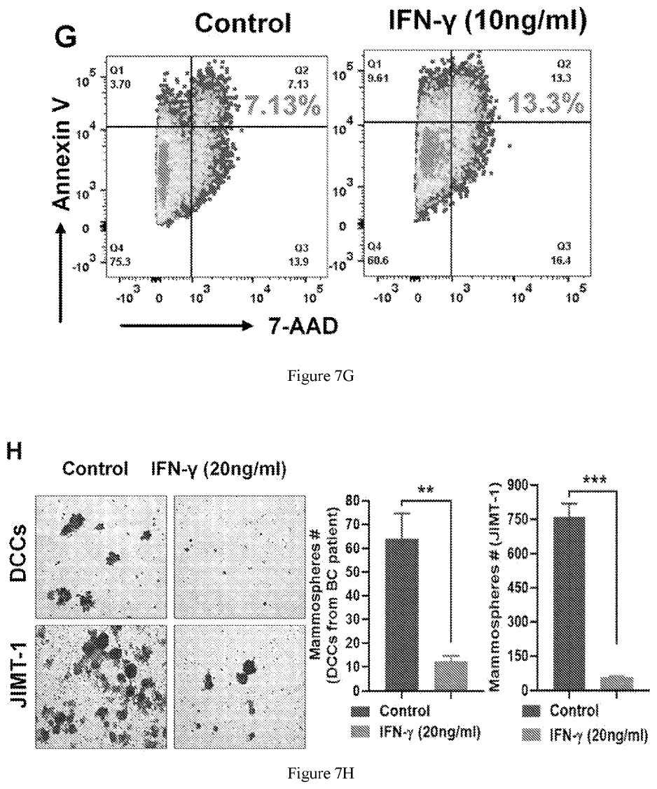

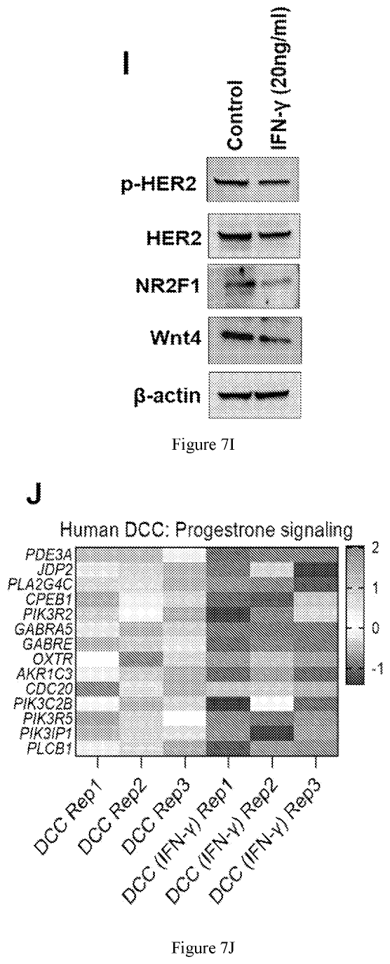

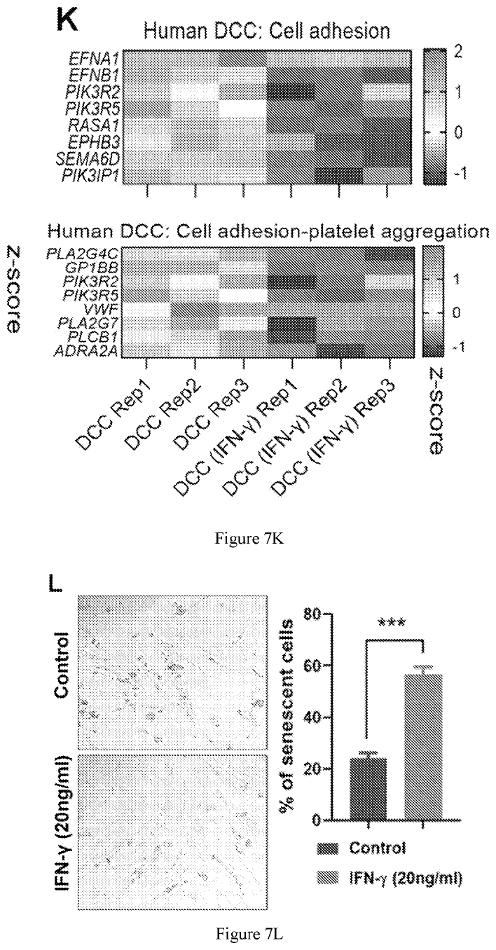

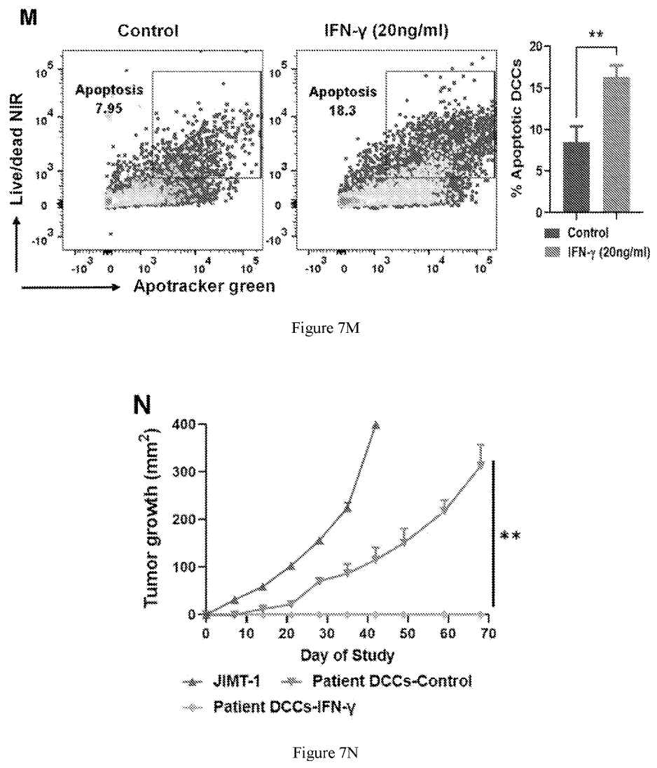

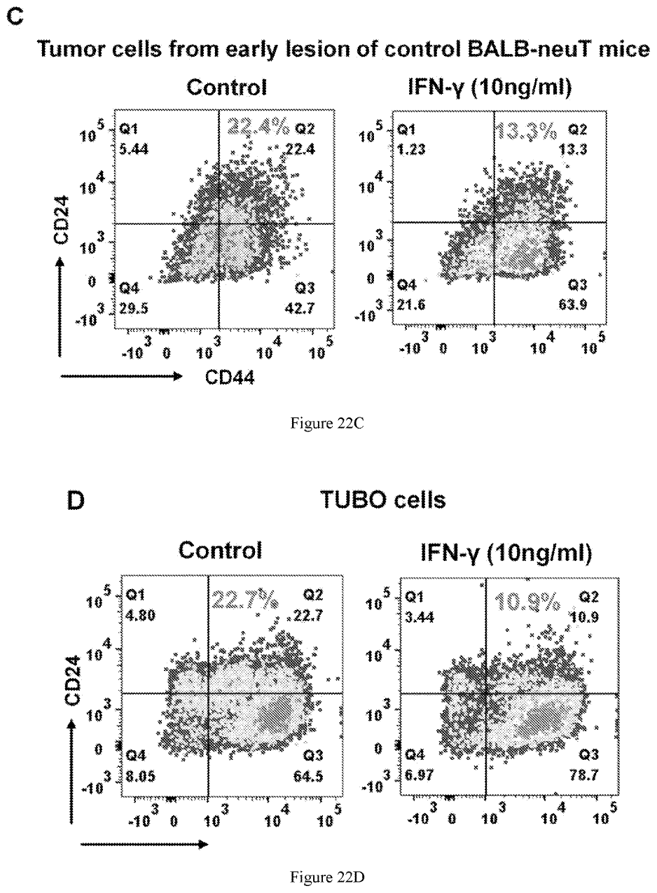

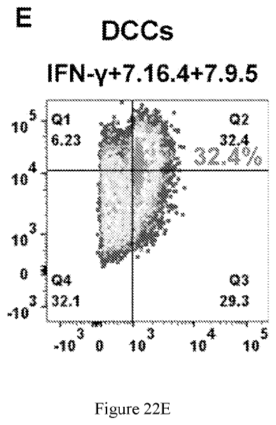

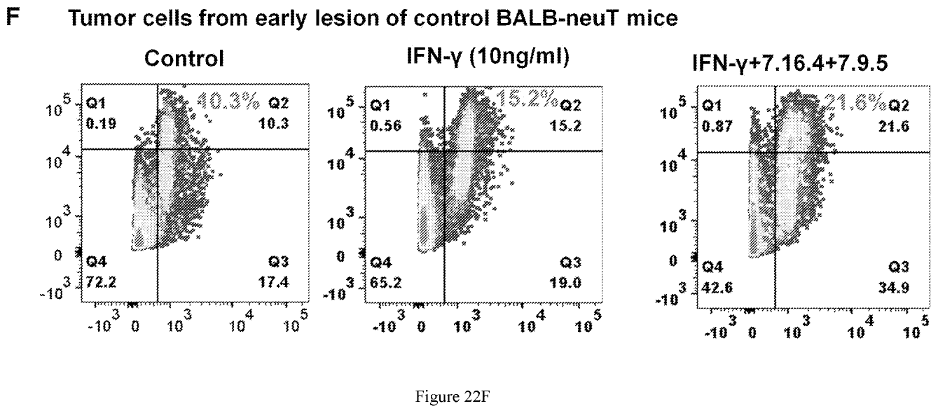

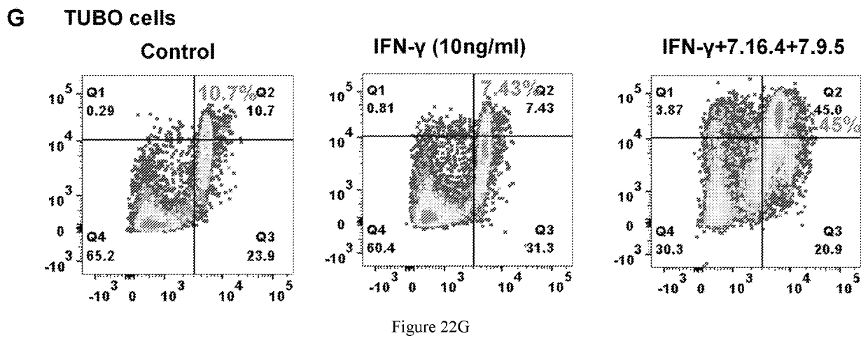

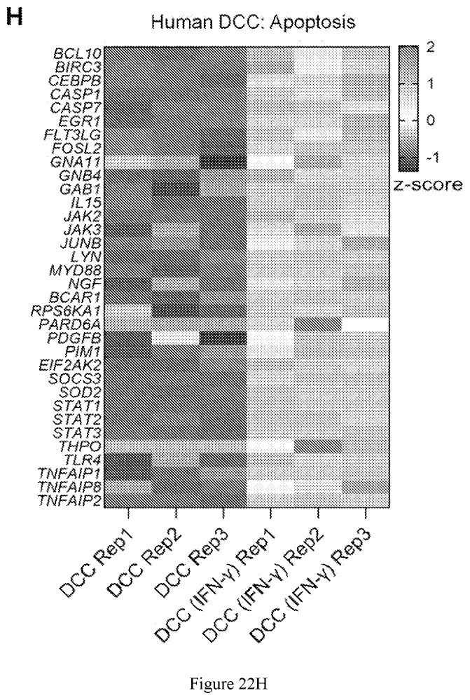

Stemness and self-renewing potential of DCCs have been shown to be responsible for resistance to conventional therapies resulting in recurrence and metastasis in BC. The therapeutic efficacy of CD4Th1 cytokine IFN-γ on sternness of DCCs isolated from BALB-neuT mice was examined. ALDEFLUOR assay revealed an enriched ALDH+ stem cell population in untreated control DCCs of BALB-neuT mice (FIG. 7A). In addition, an enriched ALDH+ stem cell population was also observed in tumor cells from BALB-neuT mice mammary lesion and TUBO cells (FIGS. 22A and 22B). Interestingly, the enriched ALDH+ stem cell population was reduced after IFN-γ treatment in DCCs of BALB-neuT mice (FIG. 7A). A similar effect on reducing the ALDH+ stem cell population was also observed in tumor cells of BALB-neuT mice mammary lesion and TUBO cells following IFN-γ treatment (FIGS. 22A and 22B). Consistent with the above finding, high levels of CD44+CD24+ double positive cancer stem cell population was detected in DCCs of BALB-neuT mice, tumor cells of BALB-neuT mice mammary lesion and TUBO cells (FIGS. 7B, 22C, and 22D). Notably, the percentage of enriched ALDH+(13.8%) and CD44+CD24+ cancer stem cell population (62.2%) was higher in DCCs of untreated control BALB-neuT mice compared to their early mammary lesion (5.19% of ALDH+ and 22.4% of CD44+CD24+ cancer stem cell population) (FIGS. 7A, 7B, 22A, and 22C). IFN-γ treatment greatly reduced the CD44+CD24+ cancer stem cell population in all three cell types (FIGS. 7B, 22C and 22D). By providing critical culturing conditions, a high number of mammospheres formation was observed (163.5±28.5) in untreated DCCs of BALB-neuT mice (FIGS. 7C and 7D), supporting the self-renewing potential of DCCs. In addition, untreated TUBO cells also generated higher mammosphere numbers (565±85) (FIGS. 7C and 7D). CD4Th1 cytokine IFN-γ treatment significantly reduced mammosphere numbers in DCCs of BALB-neuT mice (56±18; p=0.005246) and TUBO cells (103±17; p=0.000765) compared to untreated controls (FIGS. 7C and 7D). Next, the IFN-γ mediated inhibition of the self-renewing potential of DCCs from BALB-neuT mice was also accompanied by the downregulation of various gene signatures, mainly related to cell adhesion (FIG. 7E), highlighting the critical role of CD4Th1 cytokine IFN-γ in inhibiting proliferation, survival ability and tumor forming potential of DCCs. Treatment of DCCs from BALB-neuT mice with IFN-γ significantly increased the number of senescent DCCs (68.13±9.07; p=0.000617) compared to untreated control DCCs (26.11±2.59) (FIG. 7F). In addition, IFN-γ treatment increased the level of apoptotic DCCs from BALB-neuT mice which was further increased when IFN-γ was combined with anti-HER2 antibodies (FIGS. 7G and 22E). The apoptosis inducing potential of IFN-γ and its synergism with anti-HER2 antibodies was observed, in tumor cells of BALB-neuT mice mammary lesion and TUBO cells (FIGS. 22F and 22G). The study was further extended to investigate the direct efficacy of CD4Th1 cytokine IFN-γ in BM DCCs of BC patients. DCCs of HER2+ BC patients were able to form mammospheres (64±10.69) (FIG. 7H). However, CD4Th1 cytokine IFN-γ inhibited the self-renewing capability of DCCs from HER2+ BC patients, supported by a significant reduction in mammosphere numbers (12.3±2.3; p=0.0046) (FIG. 7H). This result was further confirmed in the JIMT-1 cells (759±60.28), where reduced numbers of mammospheres were observed after IFN-γ treatment (58.33±3.84; p=0.0003) (FIG. 7H). Western blot results revealed reduced expression and activation of HER2, Wnt4 and NR2F1 in IFN-γ treated DCCs when compared to untreated control DCCs of HER2+ BC patients (FIG. 7I). Subsequently, RNA sequencing identified the downregulation of various gene signatures related to progesterone signaling and cell adhesion in IFN-γ treated DCCs of HER2+ BC patients (FIGS. 7J and 7K). The effect of IFN-γ was observed on the senescence and apoptosis inducing potential in DCCs from HER2+ BC patients. As shown in FIGS. 7L and 7M, IFN-γ was able to significantly increase the level of senescence (56.42±3.08; p<0.0001 compared to untreated control; 24.12±2.04) and apoptosis (16.26±1.43; p=0.0151 compared to untreated control; 8.51±1.86) in DCCs of HER2+ BC patients. Consistent with the preceding experiments, it was also found that IFN-γ treatment upregulated various genes involved in apoptosis (FIG. 22H). Subcutaneous implantation of DCCs from HER2+ BC patients into NSG mice effectively developed tumors (FIG. 7N). Interestingly, IFN-γ treated DCCs of HER2+ BC patients failed to develop tumors in NSG mice (up to 120 days) (FIG. 7N), supporting the ability of CD4Th1 cytokine IFN-γ to inhibit DCCs tumorigenesis.

### 8. CDTh1 Cytokine IFN-γ Induced Transcriptomic Alterations in DCCs.

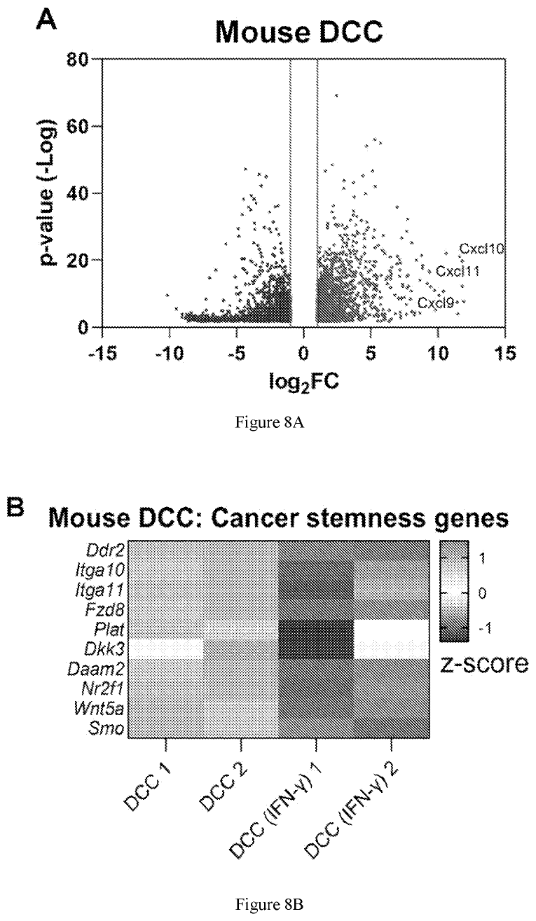

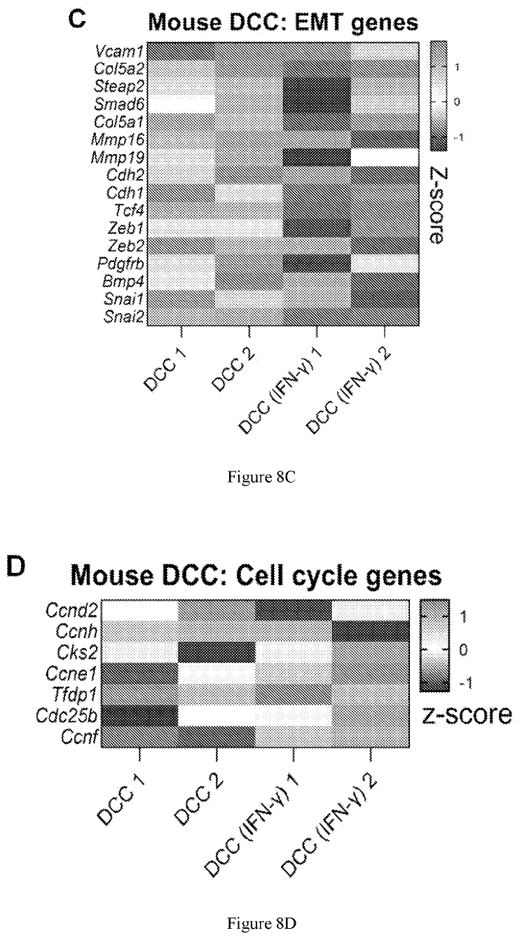

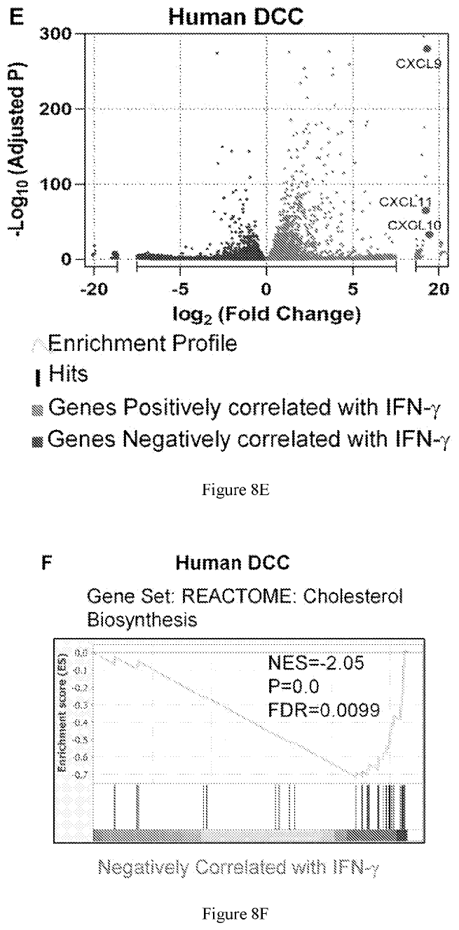

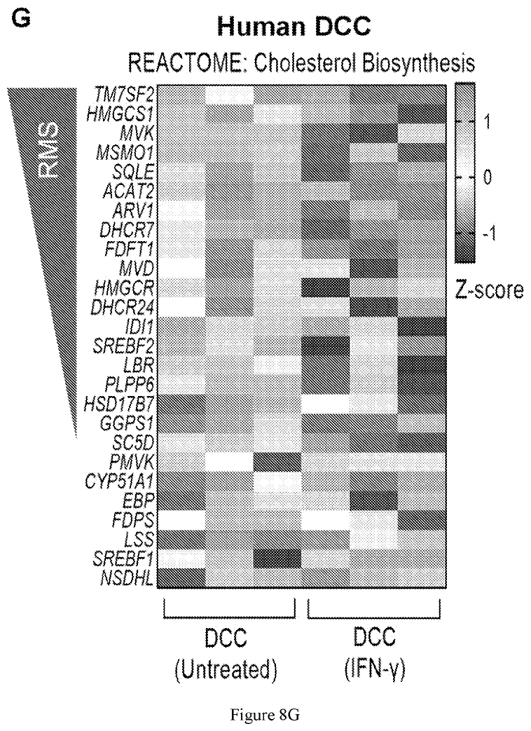

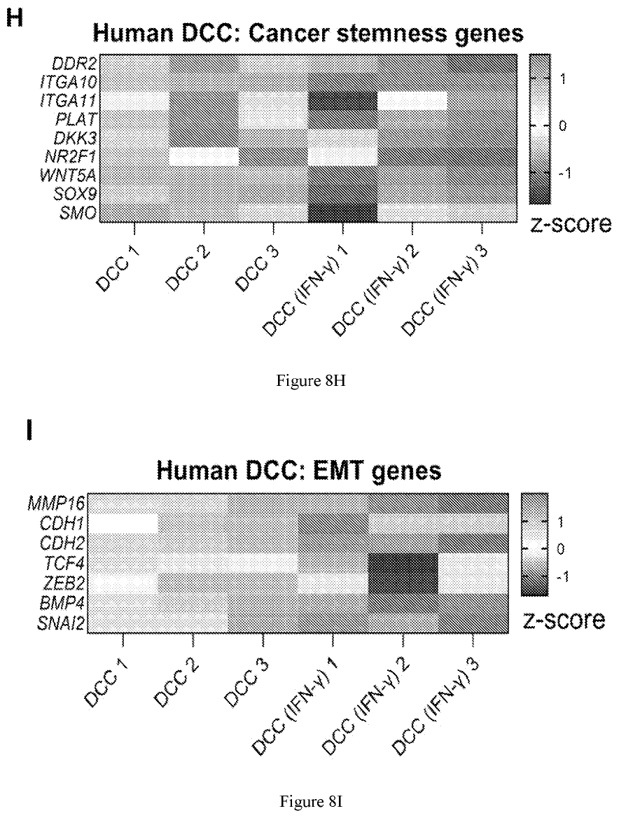

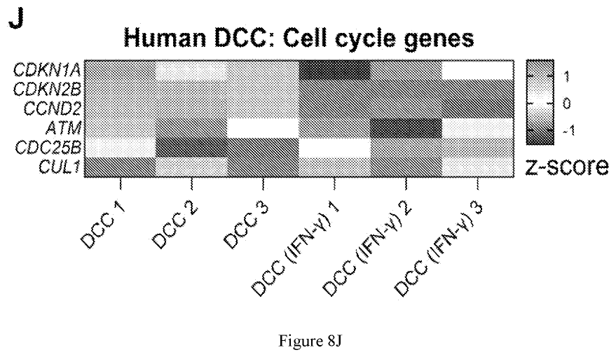

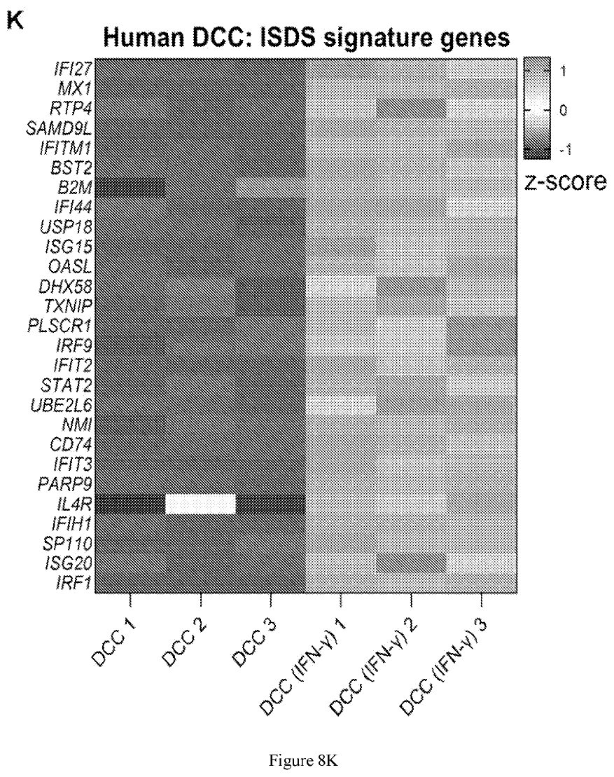

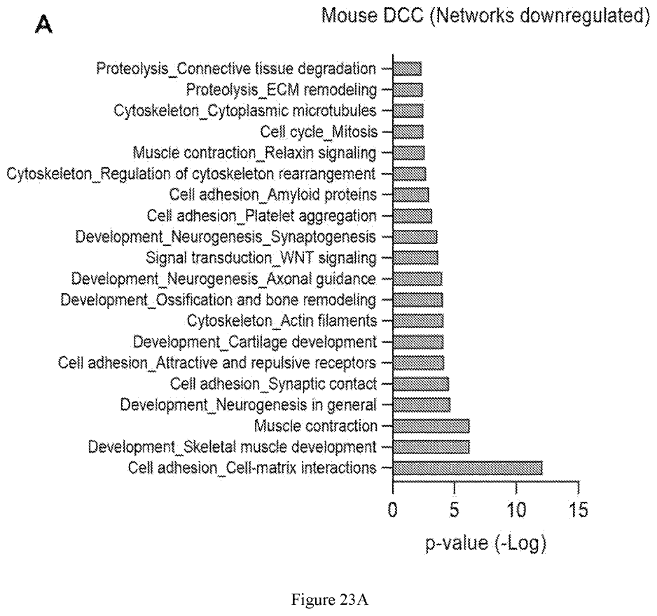

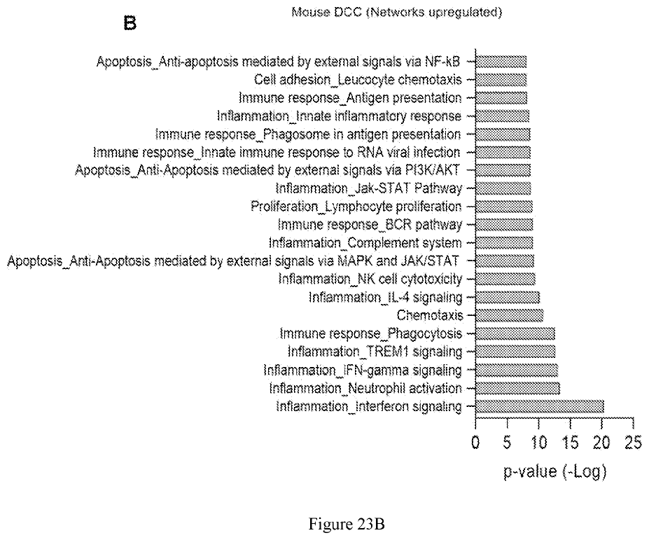

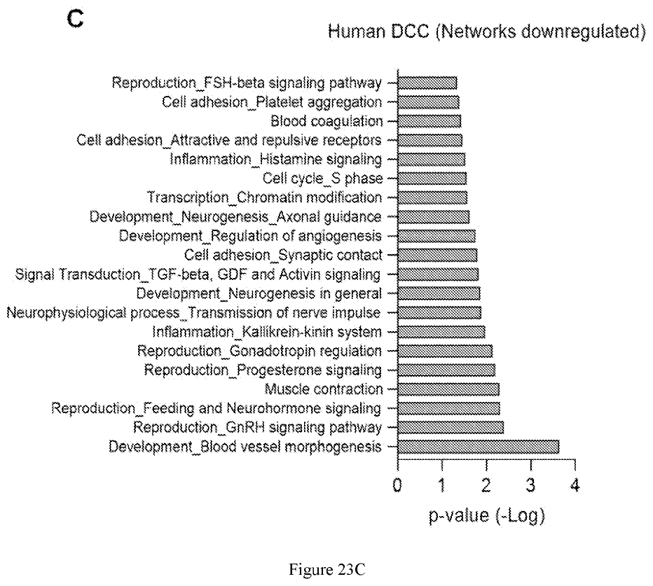

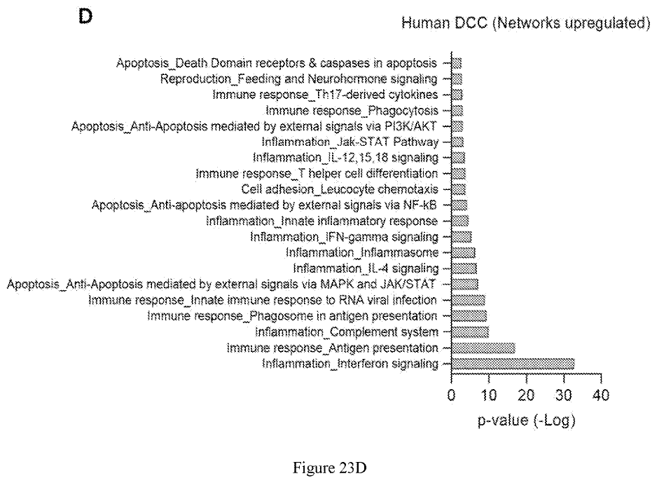

To further characterize the gene profiles of control DCCs of BALB-neuT mice and IFN-γ treated DCCs of BALB-neuT mice, RNA sequencing was performed. The differential gene expression analysis revealed that CD4Th1 cytokine IFN-γ treatment regulated various gene expression in DCCs compared to untreated control DCCs from BALB-neuT mice (FIG. 8A). Next, pathway enrichment analysis for upregulated and downregulated datasets was performed to uncover the protein response networks. As shown in FIG. 23A, the downregulated networks were mainly related to cell adhesion, Wnt signaling, cell cycle, cytoplasmic microtubules and extracellular matrix (ECM) remodeling in IFN-γ treated DCCs of BALB-neuT mice. Networks corresponding to inflammation and immune system were highly enriched in the upregulated data sets including interferon signaling, chemotaxis, antigen presentation, NK cell cytotoxicity, lymphocyte proliferation and apoptosis in IFN-γ treated DCCs of BALB-neuT mice (FIG. 23B). Next, IFN-γ treatment downregulated various cancer stemness genes and EMT genes and regulated cell cycle genes in DCCs from BALB-neuT mice when compared to untreated control DCCs of BALB-neuT mice (FIGS. 8B-8D).

The differential gene expression and pathway enrichment analyses for the RNA sequencing data of patients DCCs showed downregulation for various gene networks that were mainly associated with progesterone signaling, TGF-beta, GDF/activin signaling, cell cycle and cell adhesion in IFN-γ treated DCCs of HER2+ BC patients compared to untreated controls (FIGS. 8E and 23C). The majority of upregulated gene networks in IFN-γ treated DCCs of HER2+ BC patients were involved in interferon signaling, inflammation, antigen presentation, chemotaxis and apoptosis as compared with controls (FIGS. 8E and 23D). Strikingly, the GSEA analysis curating REACTOME database for downregulated genes revealed that cholesterol biosynthesis was the highest ranked gene set negatively correlated with IFN-γ treatment in DCCs of HER2+ BC patients (FIGS. 8F and 8G). Consistent with the modulatory effects of IFN-γ in DCCs of BALB-neuT mice, downregulation of specific cancer stemness genes, EMT genes, and cell cycle regulatory genes was also observed after IFN-γ treatment in DCCs of HER2+ BC patients (FIGS. 8H-8J). Collectively, these data provide transcriptomic evidence that anti-tumor CD4Th1 response regulates cancer stemness, EMT, cell cycle and cholesterol biosynthesis to restrain DCCs tumorigenesis.

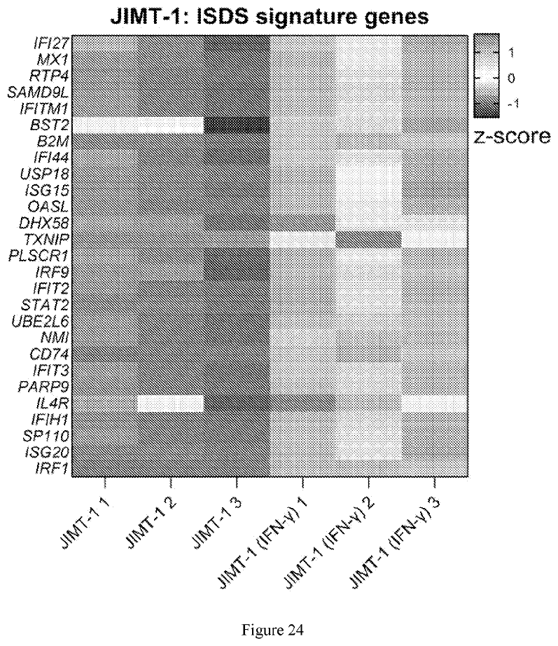

The IFN-stem cell-down signature (ISDS) gene expression has been associated with good prognosis for relapse free survival, distant metastasis free survival and overall survival in BC patients. DCCs of HER2+ BC patients have downregulated expression of ISDS gene signature (FIG. 8K). Remarkably, IFN-γ treatment upregulated the expression of 27 genes of ISDS in DCCs from the BM of HER2+ BC patients (FIG. 8K). A similar effect on the upregulation of the ISDS gene signature was observed in JIMT-1 cells after IFN-γ treatment (FIG. 24).

### a) Discussion

Detection and persistence of DCCs in the BM of BC patients have been identified as a primary source for late recurrence and distant metastasis in multiple organs. Selective targeting and eradication of DCCs in BM before succumbing to metastases can be a significant advantage for these patients. Unfortunately, no such targeted therapy presently exists, instead, intense chemotherapy in the adjuvant or neoadjuvant setting are given over a period of time with the goal to prevent recurrence in invasive BC patients. Clinical studies confirmed that these DCCs have not been effectively eradicated by chemotherapies. This study provides evidence that intratumoral priming of tumor antigen specific CD4Th1 cells enhances their migration to the BM to selectively target DCCs burden and eradicate DCC metastasis in distant organs in multiple BC spontaneous metastasis models. Interestingly, anti-tumor CD4Th1 cells mediated eradication of lung metastasis in a melanoma spontaneous metastasis model further confirmed this finding. The selective targeting of DCCs was not as extensive when CD4 T cells were absent, even in the presence of CD8 T cells, suggesting a prominent role for anti-tumor CD4Th1 cells in targeting DCCs but they may require other infiltrating immune effector cells such as NKT, NK and B cells.

Studies have shown that HER2/PR/Wnt4/RANKL signaling and HER2/Wnt4/NR2F1 signaling are involved in cancer cell dissemination in HER2+ early BC. This study demonstrates that intratumoral priming of anti-tumor CD4Th1 cells inhibited these dissemination mechanisms and restored the loss of NR2F1 in cancer cells, reducing their dissemination from the HER2+ mammary tumor lesions in HER2+ BC spontaneous metastasis model. Strikingly, theses modulatory effects were not dependent on the CD8 T cells. This study provides first clinical evidence that intratumoral priming of anti-tumor CD4Th1 cells inhibited disseminating cancer cell phenotypes HER2+PR- and HER2+PR-WNT4+RANKL+ in stage I-III invasive carcinoma of HER2+ BC patients. Previous studies have suggested that cancer cells are able to express increased Wnt4, RANKL and EMT markers, Snail and Slug, to promote EMT transition, migration, invasive potential and dissemination into distant organs in TNBC models. This study highlights that intratumoral priming of anti-tumor CD4Th1 cells decreases these migratory signatures in cancer cells and inhibits their dissemination and seeding into distant organs in a TNBC model. This may also have implications for reducing dissemination in those undergoing late cancer cell dissemination. Previous reports have suggested that NR2F1, HER2, Wnt4 and Twist can regulate dormancy, stemness and EMT transition in DCCs which render them more susceptible to a reversible programing switch from dormancy to proliferative growth, leading to disease relapse in HER2+ BC patient. Strikingly, this study demonstrates that the anti-tumor CD4Th1 response was highly effective in reducing NR2F1, HER2, Wnt4 and Twist in DCCs. Although radiation and chemotherapy can induce senescence in cancer cells and inhibit tumorigenesis, the senescence-associated secretory phenotype and reversable senescence leave them more vulnerable to contribute to a disease relapse in patients and mouse models. This study discovered an important insight that anti-tumor CD4Th1 response rendered irreversible senescence in DCCs. These senescent DCCs completely failed to grow into primary tumors and overt metastasis in distant organs in an immunocompromised mouse model.

This study provides evidence that anti-tumor CD4Th1 cells mediated targeting of DCCs and prevention of metastasis in distant organs was primary driven by IFN-γ. The precise dynamics of targeted effects of the CD4Th1 immune response on DCCs was established in the present study and it was observed that the CD4Th1 cytokine, IFN-γ, inhibited stemness and self-renewing potential and induced apoptosis in DCCs. Increased cholesterol biosynthesis has been linked to the propagation of cancer cell sternness and self-renewal and correlated with reduced recurrence free survival in BC patients. A recent study has shown that cholesterol alterations contribute to the invasion and metastasis and decreased patient survival in melanoma. Interestingly, the higher steady-state expression of cholesterol biosynthesis signatures was observed in DCCs. The CD4Th1 cytokine IFN-γ downregulated cholesterol biosynthesis, sternness and EMT signatures in DCCs and may provide new molecular evidence for the CD4Th1 response in the context of inhibiting sternness and metastasis of DCCs. IFN-γ has been shown to synergize with cholesterol inhibiting statins to effectively reduce the growth of HER2 mammary carcinoma. Further studies are warranted for anti-tumor CD4Th1 cells on the regulation of cholesterol biosynthesis in DCCs.

Loss of MHC class I, MHC class II and CD1d molecules expression has been found in various solid tumors. This study observed their downregulation of expression in DCCs, which suggests a defect in their antigen presentation machinery and a subsequent escape mechanism from T and NKT-cell mediated elimination. Interestingly, the CD4Th1 cytokine IFN-γ increased MHC class I and II expressions in DCCs. This study exhibited for the first time that the CD4Th1 cytokine IFN-γ increased CD1d expression in DCCs, supporting a novel role for the CD4Th1 response to drive NKT cell infiltration and NKT cell-mediated clearance of DCCs in distant organs. Intratumoral expression of anti-tumor immunostimulatory chemokines CXCL9, CXCL10, CXCL11, CXCL16, CX3CL1 and CCL7 can attract CD4Th1, CD8 T, B and NK cells into the TME. These immune effector cells may play a critical role in driving a stronger anti-DCC response. B cells express MHC class II and can present tumor antigens to CD4 Th1 cells. NK cells recognize cancer cells independent of MHC class I and can produce IFN-γ. NKT cells may recognize cancer cells or DCCs expressing CD1d and produce IFN-γ. Various findings have shown the beneficial therapeutic efficacy and clinical outcomes of triggering intratumoral chemokines by chemotherapy, radiation therapy and anti-PD1 immunotherapy in solid tumors. In this study, IFN-γ treated DCCs revealed an enrichment of these immunostimulatory chemokine signatures and antigen presentation associated signatures. Consistent with this assessment, the intratumoral priming of anti-tumor CD4Th1 cells also triggered infiltration of B and NK cells into the BM of BC spontaneous metastasis model.

Collectively, these observations demonstrate for the first time a clear role for the anti-tumor CD4Th1 immune response in targeting and inducing immune recognition of DCCs and generating an amplification loop for accumulation of CD4Th1, NKT, NK and B cells in the BM to eliminate DCCs and prevent overt metastasis. These data provide an important clinical implication and suggests that early cancer therapies driving anti-tumor CD4Th1 immunity may have a great potential in preventing metastasis in a wide array of cancers.

### b) Materials and Methods

a. Reagents and Antibodies

### (a) STAR Methods

### 1. Mouse Models

The BALB-HER2/neu transgenic (BALB-neuT) mice strain was a kind gift from Dr. Shari Pilon-Thomas, Moffitt Cancer Center. The breeding pair of BALB-neuT mice was obtained from Dr. Federica Cavallo, University of Torino. Mice at the age of 3-4 weeks were screened for hemizygosity (neuT⁺/neuT⁻) and only the positive littermates were used for all the experiments. Female BALB/c mice and C57BL/6 mice at 6-8 weeks of age were purchased from Charles River Laboratories. NOD/SCID-γ^{-/-} (NSG) immunodeficient mice strain was a kind gift from Dr. Shari Pilon-Thomas, Moffitt Cancer Center. B6.129S7-Ifngtm1 Ts/J IFN-γ knockout mice was purchased from Jackson laboratory. All mice were housed at the Animal Research Facility of the Moffitt Cancer Center. All animal studies were reviewed and approved by Institutional Animal Care and Use Committee (IACUC) at the University of South Florida (protocol ID: A4100-01). All experiments were conducted in accordance with the National Institute of Health (NIH) guidelines and recommendations for the care and use of laboratory animals.

### 2. HER2-DC1 Intramammary Gland Delivery in BALB-neuT Mice

BALB-neuT mice were monitored for spontaneous micro-invasion in their mammary glands at 8 weeks of age by magnetic resonance imaging (MRI). The MHC class II HER2 peptides pulsed type 1 polarized dendritic cells (HER2-DC1, 1×10⁶ cells/mouse) were injected into the intramammary gland facilitated by ultrasound guidance. BALB-neuT mice received HER2-DC1 intramammary gland injection into their fifth mammary gland once weekly for six weeks. Another group of BALB-neuT mice received subcutaneous delivery of HER2-DC1 once weekly for six weeks. DC1 processing, pulsing with MHC class II recognizing multi-epitope peptides p5 (ELAAWCRWGFLLALLPPGIAG), p435 (IRGRILHDGAYSLTLQGLGIH) and p1209 (SPPHPSPAFSPAFDNLYYWDQ) from rat HER2/neu oncogene and vaccine preparation was carried out as previously described. Untreated control BALB-neuT mice were administered sterile PBS injected into the mammary gland. The spontaneous tumor growth development was continuously monitored by MRI at various intervals until 24 weeks of age.

### 3. MRI Imaging

MRI imaging was performed using a 7-Tesla horizontal MRI scanner (Bruker BioSpin, Ettlingen, Germany) with a 35 mm Litzcage coil (Doty Scientific). In preparation for imaging, experimental BALB-neuT mice were anesthetized using 2% isoflurane supplied in an induction chamber along with 1.5-liter/min oxygen delivery and transferred for scanning. BALB-neuT mice were secured in a mouse coil chamber and placed in the scanner maintaining the same ventilation supply through a nose cone. The mice respiration rate was maintained between 40-60 breaths per min while scanning. The body core temperature was maintained at 37° C. with a MR-compatible Small Rodent Heater System (SAII®, SA Instruments, Stony Brook, NY) and monitored over time. Anatomical T2-weighted coronal images were acquired for each mouse with a TurboRARE sequence. Typical acquisition parameters were as follows—1.2 mm slice thickness, 75×35 mm² field of view (FOV), 512×256 matrix size and 19 slices using echo time/repetition time (TR/TE)=4513/38 ms.

### 4. CD4 and CD8 T Cells Depletion Experiments in BALB-neuT Mice

CD4 and CD8 T cells depletion experiments were performed using anti-mouse CD4 (Cat. No. BP0003-1, clone GK1.5, BioXCell, West Lebanon, NH) and anti-mouse CD8a (Cat. No. BE0061, clone 2.43, BioXCell) antibodies respectively. BALB-neuT mice at 7 weeks of age were monitored for spontaneous mammary carcinoma by MRI and confirmed for no evidence of microinvasion in their mammary glands. These mice were then injected with 300 μg of anti-CD4 or anti-CD8 intraperitoneally (i.p.) twice weekly, until the experimental end point. HER2-DC1 delivery was carried out as described above. Untreated control BALB-neuT mice were injected with rat IgG2b isotype (Cat. No. BP0090, clone LTF-2, BioXCell) intraperitoneally. Experimental BALB-neuT mice were examined for mammary carcinoma development by MRI periodically until 24 weeks of age.

### 5. HER3-DC1 Intratumoral Delivery in 4T1 Spontaneous Metastasis Model

BALB/c mice were orthotopically implanted with 4T1 cells (50,000 cells) into the mammary fat pad (MFP) and microscopic tumor growth was verified on day 3 by ultrasound. Mice bearing microscopic tumors in the MFP were administered intratumoral HER3-DC1 twice a week for three weeks. Intratumoral HER3-DC1 injection was performed as previously described. HER3-DC1 was prepared by pulsing DC1 with MHC class IerI recognizing HER3 multi-epitope peptides as follows—extra cellular domain (ECD) p12 (CEVVMGNLEIVLTGH), ECD p81 (SWPPHMHNFSVFSNL), ECD p84 (TTIGGRSLYNRGFSL), ECD p91 (AGRIYISANRQLCYH) and intra cellular domain (ICD) p38 (VADFGVADLLPPDDK), ICD p41 (QLLYSEAKTPIKWMA), ICD p52 (VPDLLEKGERLAQPQ), p86 ICD (GCLASESSEGHVTGS), and ICD p89 (EAELQEKVSMCRSRS). The mice tumors were measured twice weekly using digital calipers. At the endpoint, the experimental mice were sacrificed, the primary tumors and distant organs lung and liver were collected for molecular analysis and spontaneous metastases detection. In another experiment, 4T1 tumor bearing mice were treated with intratumoral HER3-DC1, subcutaneous HER3-DC1, intratumoral immature HER3-iDC, unpulsed DC1 and intratumoral HER2-DC1 (DC1 targeting irrelevant tumor antigen). These mice were examined for primary tumor growth and spontaneous metastasis.

For the CD4 and CD8 T cell depletion experiment, mice were injected with anti-CD4 or 10 anti-CD8 i.p. twice a week beginning three days prior to 4T1 cell inoculation in the MFP and continued until the end point. The rat IgG2b isotype control antibody was injected into non-depleted mice. For in vivo IFN-γ neutralization, mice were injected i.p. with 200 g of anti-mouse IFN-γ antibody (Cat. No. BE0055, clone XMG1.2, BioXCell) or rat IgG1 isotype control, anti-horseradish peroxidase (Cat. No. EB0088, clone HRPN, BioXCell) every third day until the endpoint. The HER3-DC1 treatment was performed as detailed above.

### 6. HER3-DC1 Intratumoral Delivery in a B16F10 Melanoma Spontaneous Metastasis Model

C57BL/6 mice or IFN-γ knockout mice were subcutaneously implanted with B16F10 cells (1×10⁶ cells) in the left flank. On day 3, mice bearing microscopic tumors were administrated intratumoral HER3-DC1 and these injections continued twice a week for three weeks. Mice were monitored and tumors were measured periodically. Lungs were collected at the endpoint for spontaneous metastases detection.

### 7. Adoptive Transfer of Anti-Tumor CD4Th1 Cells in a HER2+ Breast Cancer Model

CD4 T cells were isolated from intratumoral HER2-DC1 treated HER2+ BC bearing mice or native BALB/c mice using EasySep mouse CD4+ T cell isolation kit (Cat. No: 19852, Stemcell Technologies) following manufacturer's instructions. Anti-HER2 CD4Th1 cells were then expanded by co-culturing with HER2-DC1 in the presence of IL-2 (Cat. No: 162-8221, Prometheus Laboratories, San Diego, CA) and IL-7 (Cat. No: 217-17, Peprotech, Cranbury, NJ) cytokines. BALB/c mice were orthotopically implanted with HER2+ TUBO cells (30,000 cells) into the MFP. Mice bearing orthotopic palpable tumors were treated with intratumoral anti-HER2 CDTh1 cells (1×10⁶ cells), intratumoral non-specific CD4Th1 cells (1×10⁶ cells), intratumoral HER2-DC1 or combination therapy weekly for six weeks. In another experiment, BALB/c mice were subcutaneously implanted with HER2+ TUBO cells (2.5×10⁵ cells) into the left flank. After tumors were palpable, mice were administered intratumoral HER2-DC1 with or without i.v. injection of anti-HER2 CD4Th1 cells. Mice were monitored for tumor growth and tumors were measured twice weekly using digital calipers.

### 8. Immune Cell Phenotyping by Flow Cytometry

Experimental BALB-neuT mice at week 16 were sacrificed by euthanasia. The mammary glands positive or negative for tumors were collected separately and processed into single cell suspensions as previously described. 1×10⁶ cells were stained with Live/Dead Zombie near IR (Cat. No. 423106, BioLegend, San Diego, CA) for 30 minutes in the dark at room temperature. Cells were then surface stained with lymphoid immune cell phenotyping 10 antibodies: anti-CD45 BUV 395 (Cat. No. 564279, Clone 30-F11, BD Biosciences, San Jose, CA), anti-CD3 APC (Cat. No. 553066, Clone 145-2C11, BD Biosciences), anti-CD4 PerCP-Cy5.5 (Cat. No. 550954, Clone RM4-5, BD Biosciences), anti-CD8 Pac Blue (Cat. No. 558106, Clone 53-6.7, BD Biosciences), anti-CD62L BUV737 (Cat. No. 612833, Clone MEL-14, BD Biosciences), anti-CD44 FITC (Cat. No. 553133, Clone IM7, BD Biosciences), anti-CD49b or DX-5 pan NK PE (Cat. No. 108908, Clone DX5, BioLegend) and anti-CD19 PE/Cyanine7 (Cat. No. 115520, Clone 6D5, BioLegend) for 30 minutes on ice in the dark and washed with FACS buffer. Data were acquired on an LSRII flow cytometer (BD Biosciences) using FACS Diva software ((BD Biosciences). FACS data were analyzed with FlowJo software. Gating strategy for identifying various immune effector cells and immune profiling was performed as previously described.

### 9. Intracellular IFN-γ Staining by Flow Cytometry.

CD4 T cells were isolated from tumors and BM of control and intratumoral HER3-DC1 treated 4T1 tumor bearing mice. Isolated CD4 T cells were then co-cultured with HER3-DC1 (DC1 targeting TNBC tumor antigen) or HER2-DC1 (DC1 targeting irrelevant tumor antigen) (10:1 ratio) over night. eBioscience Protein Transport Inhibitor Cocktail containing Brefeldin A and Monensin (Cat. No: 00498003, ThermoFisher scientific) was added to inhibit intracellular protein transport for 6 hours. Cells were harvested and surface stained for anti-CD3, anti-CD4 and anti-CD8 as described above. Intracellular IFN-γ staining was performed using the eBioscience Intracellular Fixation & Permeabilization Buffer kit (Cat. No: 88882400, ThermoFisher scientific) following the manufacturer's protocol and stained for anti IFN-γ (Cat. No: 554412, Clone XMG1.2, BD Biosciences). Data acquisition was performed as described above.

### 10. IFN-γ Quantification by ELLA Automated ELISA Instrument

The HER2-DC1 mediated anti-HER2 Th1 response in BALB-neuT mice was examined using the Simple Plex mouse IFN-γ assay kit (Cat. No. SPCKB-MP-001945, Bio-Techne, Minneapolis, MN). Spleens and tumor draining lymph nodes (TDLNs) were collected from untreated and HER2-DC1 treated BALB-neuT mice at week 16 and single cell suspensions were prepared as described previously. Splenocytes were stimulated with or without MHC class II recognizing rat HER2/neu peptides p5, p435 and p1209 individually for 72 hours. TDLNs were co-cultured with or without HER2-DC1 (DC1 individually pulsed with p5, p435 and p1209) at a 1:10 ratio for 72 hours. Following incubation, culture supernatants were centrifuged at 1000 rpm for 5 minutes. Supernatants were collected and measured for IFN-γ using a Simple Plex mouse IFN-γ assay kit and run on an ELLA automated ELISA instrument (Bio-Techne, Minneapolis, MN).

### 11. Transwell Migration Assay

The transwell migration assay was performed using Corning 12 mm Transwell with 3-μm pore permeable polycarbonate membrane inserts (Cat. no. 07-200-157, Fisher Scientific). Aanti-HER2 CD4Th1 cells were co-cultured with HER2-DC1 or unpulsed DC1 in the presence or absence of IFN-γ neutralizing antibody or isotype control antibody in the top chamber of the transwell plate. The DCCs from control BALB-neuT mice or HER2+ TUBO cells were seeded in the bottom chamber of the transwell plate in the presence or absence of IFN-γ neutralizing antibody or isotype control antibody. After 72 hours of co-culture, the top chamber was removed and the bottom chamber containing DCCs or HER2+ TUBO cells were assessed for senescence-associated β-galactosidase (SA-β-gal) activity using cellular senescence assay kit (Cat. No. KAA002, Fisher Scientific) per the manufacturer's instructions.

### 12. Histopathology and Immunohistochemistry

The collected tumor tissue, lung, liver and brain were fixed with 10% formalin for 48 to 72 hours then transferred to 70% ethanol. Tissue paraffin embedding, slide sectioning and hematoxylin-eosin (H&E) staining were performed as previously described. Immunohistochemistry (IHC) staining was done with the following antibodies: anti-HER2 (Cat. No. 4290, Cell Signaling Technology), anti-Wnt4 (Cat. No. bs-6134R, BIOSS, Woburn, MA), 30 anti-RANKL (Cat. No. ab45039, ab216484, Abcam, Cambridge, MA), anti-NR2F1 (Cat. No. ab224272, Abcam), anti-Snail+Slug (Cat. No. ab180714, Abcam) using the Ventana Discovery XT automated system (Ventana Medical Systems, Tucson, AZ) following manufacturer's instructions. IHC for anti-PR (Cat. No. ab131486, Abcam) was performed using a Leica Bond RX automated system (Leica Biosystems, Buffalo Grove, IL) following manufacturer's protocol. The IHC double staining for anti-HER2 (Cat. No. 4290, Cell Signaling Technology) and anti-cytokeratin 8/18 (Cat. No. MA5-32118, Thermo Scientific) or anti-HER3 (Cat. No. 12708, Cell Signaling Technology) and anti-Pan cytokeratin (Cat. No. NB600-579, Novus Biologicals, Littleton, CO) was performed using Ventana Discovery XT automated system (Ventana Medical Systems, Tucson, AZ) as per the manufacturer's instructions. All stained slides were scanned and analyzed using Leica Aperio™ AT2 scanner (Leica Biosystems, Vista, CA) at the Microscopy Core Facility of the Moffitt Cancer Center.

### 13. DCCs Detection by Immunofluorescence and Flow Cytometry

Bone marrow was collected from femurs and tibia of experimental BALB-neuT mice at week 16 or experimental BALB/c mice bearing 4T1 tumors or naïve BALB/c mice as described previously. Briefly, the bone marrow was flushed with a PBS using a 26-G needle, then the red blood cells were lysed with ACK lysis buffer and re-suspended in RPMI 1640. Cells were placed on poly-D-lysine (P6407, Sigma-Aldrich) coated cell culture chamber (Cat. No: 80841, Ibidi) and incubated for 2 hours at 37° C. in CO₂ incubator. The attached cells were fixed using ice-cold 4% paraformaldehyde and permeabilized with 0.2% Triton X. Slides were blocked with 5% bovine serum albumin (BSA) to reduce background staining and then incubated with anti-HER2 (Cat. No: BE0277, clone: 7.16.4, BioXCell), anti-cytokeratin 8/18 (Cat. No: ab53280, Abcam) and anti-Ki-67 (Cat. No: AF7649, R&D Systems). Goat anti-mouse FITC (Cat. No: NC9352374, Fisher Scientific), goat anti-rabbit AF594 (Cat. No: 8889S, Cell Signaling Technology) and donkey anti-sheep AF647 (Cat. No: ab150179, Abcam) were used as secondary antibodies. Stained slides were mounted in VECTASHIELD antifade mounting medium with DAPI (Cat. No: H-1200-10, Vector Laboratories) and imaged with Zeiss Apotome.2 fluorescence microscope (Zeiss, Thornwood, NY). For flow cytometry, single cell suspensions of bone marrow or lungs (1×10⁶ cells) were incubated with Live/Dead Zombie near IR as specified above. Cells were stained with anti-CD45 BUV395 (Cat. No. 564279, Clone 30-F11, BD Biosciences), anti-HER2 APC (Cat. No: FAB6744A-100UG, R&D Systems) or anti-EpCAM PE (Cat. No: 118206, Biolegend), anti-cytokeratin 8/18 FITC (Cat. No: ab52459, Abcam) and anti-Ki-67 BV786 (Cat. No. 563756, BD Biosciences) or anti-Ki-67 PE (Cat. No: NB110-89717PE, Fisher Scientific) for 30 minutes in 4° C. Cells were run on an LSRII flow cytometer, and the data was analyzed with FlowJo software. In addition, indirect flow cytometry staining on DCCs was also performed using primary and secondary antibodies mentioned above.

14. DCCs Isolation from BALB-neuT Mice and Culture

Bone marrow of experimental BALB-neuT mice was collected and single cell suspension was prepared as specified above. CD45⁺ immune cells were depleted using EasySep mouse CD45 positive selection kit (Cat. No: 18945, Stemcell Technologies) following manufacturer's instructions. Ep-CAM positive DCCs were then isolated from the CD45⁻ cells using the MojoSort mouse CD326 (Ep-CAM) selection Kit (Cat. No: 480142, Biolegend) following manufacturer's instructions. The isolated DCCs were cultured in the presence of 30 ng/ml EGF (Cat. no. 315-09, PeproTech, Cranbury, NJ) and 20 ng/ml FGF-basic (Cat. no. 450-33, PeproTech) at 37° C. in a CO₂ incubator.

### 15. Tumor Cell Lines, Culture and Treatments

The mouse TUBO cell line (a kind gift from Dr. Wei Zen Wei, Wayne State University) was cloned from a spontaneous primary mammary carcinoma of BALB-neuT mice. JIMT-1, a human breast cancer cell line was obtained from the DSMZ (Cat. no: ACC589). Human breast cancer cell lines BT474 (Cat. No: HTB20) and HCC1954 (Cat. No: CRL-2338) were purchased from ATCC. The mouse B16F10 melanoma cell line was a kind gift from Dr. Dr. Shari Pilon-Thomas, Moffitt Cancer Center. Mammary glands with early tumor lesion were collected from BALB-neuT mice between 14-16 weeks and single cell suspensions were prepared as previously described. These early mammary tumor lesion cells were also used for in vitro experiments. Cells were cultured as previously described. Cells were treated with IFN-γ (10 ng/ml) or TNF-α 20 (1 ng/ml), anti-HER2 antibodies 7.16.4 (5 ug/ml) and 7.9.5 (5 ug/ml) or in combination for 24-72 hours. The 7.16.4 clone that mimics trastuzumab was purchased from BioXCell (Cat. No: BE0277) and the 7.9.5 clone that mimics pertuzumab was a kind gift from Dr. Mark I. Greene, University of Pennsylvania.

### 16. Experimental Primary and Metastasis Assays Using NSG Mice Model

Bone marrow pool (5×10⁶) or equal number of isolated DCCs (2.5×10⁵) from control and HER2-DC1 treated BALB-neuT mice between 16-18 weeks, or in vitro expanded DCCs (2.5×10⁵) from control BALB-neuT mice, or TUBO cells (2.5×10⁵) were re-suspended in PBS, plus an equal volume of CultrexBME (Cat. no: 3432-005-01, R&D Systems, Minneapolis, MN) and mixed with EGF (5 ug) and bFGF (5 ug). Cells were then injected subcutaneously in the NSG mice as previously described. Upon appearance of palpable tumors, the tumor growth in NSG mice was measured once in a week with vernier caliper. After monitoring tumor growth for three months, mice were euthanized and lung, liver and brain were collected to examine micrometastases.

### 17. Western Blot

Cell lysates or tissue lysates were prepared using RIPA buffer (Cat. No. 20-188, Millipore) containing a protease inhibitor cocktail (Cat. No. P8340-1ML, Sigma-Aldrich) and phosphatase inhibitors (Cat. No. A32957, Pierce). The protein concentration was determined using the Bradford protein assay (Cat. No. 5000006, Bio-Rad, Hercules, CA). 20-40 ug of total protein were resolved in 4-12% SDS-PAGE gels and transferred onto PVDF membrane (Cat. No. IPVH00010, Millipore) using eBlot® L1 wet transfer system (GenScript, Piscataway, NJ). Subsequently, the membranes were blocked with 5% BSA in TBST, and immunoblotted with primary and secondary antibodies. The membranes were visualized using Pierce ECL Western Blotting Substrate (Cat. No. 32106, ThermoFisher Scientific). Bands were visualized and imaged using Odyssey Fc imaging system (LI-COR Biosciences, Lincoln, NE).

The primary antibodies used were anti-HER2 (Cat. No. 2165S, Cell Signaling Technologies or ab131490, Abcam), anti-pHER2 Tyr1221/1222 (Cat. No. 2243S, Cell Signaling Technologies), anti-PR (ab2765, Abcam or 8757S, Cell Signaling Technologies), anti-Wnt4 (ab91226, Abcam), anti-RANKL (ab45039, Abcam), anti-NR2F1/COUP-TFI (Cat. no. 6364S, Cell Signaling Technologies), anti-Twist (ab50887, Abcam), anti-cleaved caspase 3 (Asp175; Cat. No. 9661S, Cell Signaling Technologies) and anti-β actin (Cat. No. 4967S, Cell Signaling Technologies).

### 18. Mammosphere Formation Assay

A mammosphere formation assay was performed as previously described. DCCs, TUBO cells, or JIMT-1 cells were passed through a 25-G needle 10 times and filtered through a 70-μm cell strainer. The enriched single cells were plated in Nunclon Sphera 6-Well Plates (Cat. no. 174932, Thermo Fisher Scientific) or Corning costar ultra-low attachment plates (Cat. no. 7200601, Fisher Scientific). Cells were cultured in PromoCell 3D Tumorsphere medium XF (Cat. no. C-28070, Sigma Aldrich) and supplemented with 1:50 B27 Plus Supplement (Cat. no. A3582801, Thermo Fisher Scientific) and 20 ng/ml EGF. Cells were then treated with various treatment conditions as specified above. Cells were incubated for 14 days and mammospheres were counted.

### 19. Transwell Invasion Assay

A transwell invasion assay was performed using Corning 6.5 mm Transwell with 8-μm pore permeable polycarbonate membrane inserts (Cat. no. 07-200-150, Fisher Scientific) as previously described. Inserts of the Transwell were coated with 30% CultrexBME. Freshly isolated early mammary tumor lesion cells (4×10⁵) from BALB-neuT mice or TUBO cells (4×10⁴) were resuspended in 200 μl of RPMI-1640 free of FBS and seeded on the coated CultrexBME layer in top chamber. RPMI-1640 containing FBS was then added in the lower chamber. Cells in the top chamber were stimulated with, or without, 10 nM progesterone (Cat. no. P6149, Sigma Aldrich). Cells were treated with various treatment conditions for 72 hours as specified above. Lower chambers containing culture medium were also supplemented with similar treatment conditions in the absence of progesterone stimulation. After incubation, inserts were removed from the Transwell plate and cells were fixed with methanol for 10 min at −20° C. Inserts were stained with crystal violet and non-invasive cells were removed. Inserts were then visualized under the light microscope at 4× magnification and invasive cells were counted from 3 different observed fields.

### 20. Cellular Senescence Assay

DCCs from bone marrow of experimental BALB-neuT mice or in vitro expanded DCCs treated with various treatment conditions were assessed for SA-3-gal activity using cellular senescence assay kit (Cat. no. KAA002, Fisher Scientific) per the manufacturer's instructions. Cells were visualized and imaged using a bright-field microscope. The SA-R-gal positive (blue) cells or SA-β-gal negative cells were counted, and the percentage of SA-β-gal-positive cells were determined.

21. Aldefluor Assay and CD44⁺CD24⁺ Cancer Stem Cell Staining by Flow Cytometry

DCCs from the bone marrow of BALB-neuT mice were treated with various treatment conditions for 48 hours as detailed above. Cells were collected using StemPro Accutase Cell Dissociation Reagent (Sigma Aldrich), resuspended in PBS and filtered through a 70-μm cell strainer. An Aldefluor assay was performed using the Aldefluor kit (Cat. no. 01700, Stemcell Technologies) following manufacturer's instructions. For CD44+CD24+ cancer stem cell staining, cells were incubated with Live/Dead Zombie near IR and stained with anti-CD44 Pacific Blue (103020, Biolegend) and anti-CD24 PE (138504, Biolegend). Stained cells were acquired using LSRII flow cytometer (BD Biosciences) and FACS data was analyzed using FlowJo software (Tree Star).

### 22. Apoptosis Assay

The detection of apoptosis in DCCs (from the bone marrow of BALB-neuT mice), early mammary tumor lesion cells (from BALB-neuT mice) and TUBO cells after various treatments was performed using the APC Annexin V apoptosis detection kit with 7-AAD (Cat. no. 640930, Biolegend) following manufacturer's protocol. Stained cells were acquired using LSRII flow cytometer (BD Biosciences) and FACS data was analyzed using FlowJo software (Tree Star).

### 23. RNA Sequencing

Total RNA was extracted using the RNeasy Mini Kit (Cat. no. 74104, Qiagen, Germantown, MD) following manufacturer's instructions. RNA was quantitated with the Qubit Fluorometer (ThermoFisher Scientific, Waltham, MA) and screened for quality on the Agilent TapeStation 4200 (Agilent Technologies, Santa Clara, CA). The samples were then processed for RNA sequencing using the NuGEN Universal RNA-Seq Library Preparation Kit with NuQuant (Tecan Genomics, Redwood City, CA). 100 ng of RNA was used to generate cDNA and a strand-specific library following the manufacturer's protocol. Quality control steps were performed including TapeStation size assessment and quantification using the Kapa Library Quantification Kit (Roche, Wilmington, MA). The final libraries were normalized, denatured, and sequenced on the Illumina NextSeq 2000 sequencer with the P3-200 cycle reagent kit to generate approximately 100M million 105-base read pairs per sample (Illumina, Inc., San Diego, CA).

After the initial quality assessment and adaptor trimming with cutadapt v1.8.1, the RNA sequencing reads from mice were subjected to various pre- and post-alignment QC measures before being mapped against the reference genome mm10 using Tophat 2.0.13. Gene-level quantification was performed using HTSeq 0.6.1 by summing the raw counts of reads aligned to each gene's region. For human RNA sequencing data, the reads were aligned against the human genome HG19 using STAR-2.5.3a. Gene expression was evaluated by calculating the read count at the gene level with RSEM and Gencode gene model v30. Normalized gene expression was then calculated using the R/Bioconductor package DESeq2 v1.6.3.

### 24. Analysis of RNA Sequencing Data

Enrichment analyses on expression datasets were performed using gene set enrichment analysis (GSEA) and MetaCore (Clarivate Analytics, Philadelphia, PA, USA). GSEA was performed on ranked gene lists based on their differential gene expression and adjusted P-values via REACTOME database. To examine the enrichment of specific gene networks, MetaCore-specific GO analysis was performed on curated gene lists of control and treatment samples for the following cutoffs: 2-fold change, and adjusted p-value<0.05.

25. DCCs Isolation from Bone Marrow Aspirates of Breast Cancer Patients

Newly diagnosed HER2-+ ductal carcinoma in situ (DCIS) and early invasive BC patients (prior to any treatment) with a high risk for residual disease or recurrence tested by DCISionRT (Preludedx) were recruited to participate in this feasibility study at the H. Lee Moffitt Cancer Center and Research Institute. Informed written consent was obtained from all participants (6 patients). The study protocol was reviewed and approved by the institutional review board (IRB) at the H. Lee Moffitt Cancer Center and Research Institute (protocol ID: MCC21045). Bone marrow aspirates were collected from the anterior or posterior superior iliac crest at the time of surgery (prior to any treatment) while patients were under anesthesia and transferred into EDTA containing collection tubes. DCCs isolation was performed within 24 hours after bone marrow aspiration. Red blood cells were lysed with ACK lysis buffer prior to DCC isolation. EasySep Direct Human CTC Enrichment Kit (Cat. No: 19657, Stemcell Technologies) was used to deplete all immune cells, hematopoietic cells, platelets and fibroblasts in the bone marrow. The enriched cells were then processed to isolate EpCAM positive DCCs using a EasySep Human EpCAM Positive Selection Kit II (Cat. no. 17846, Stemicell Technologies) following manufacturer's instructions. Immunofluorescence was then performed using anti-HER2 (Cat. No: BE0277, clone: 7.16.4, BioXCell) and anti-cytokeratin 8/18 (Cat. No: 4546S, Cell Signaling Technologies; Cat. No: ab53280, Abcam) primary antibodies. Goat anti-mouse FITC (Cat. No: NC9352374, Fisher Scientific) and goat anti-rabbit AF594 (Cat. No: 8889S, Cell Signaling Technology) were used as secondary antibodies.

### 26. Patient DCCs Culture, Treatment and Various Analyses

The isolated patient DCCs were cultured and expanded in RPMI-1640 supplemented with 10% FBS, 30 ng/ml rhEGF (Cat. no. 236-EG-200, R&D Systems) and 20 ng/ml rhFGF-basic (Cat. no. 233-FB-025, R&D Systems) with 5% CO₂ supply. DCCs were treated with or without IFN-γ (20 ng/ml) for 24-72 hours. For the tumorigenicity assay, patient DCCs (30,000 cells; treated with or without IFN-γ) or JMIT-1 (3×10⁶) were implanted in NSG mice as detailed above and were monitored for tumor growth. In addition, a mammosphere formation assay, cellular senescence assay and western blot for HER2, phaspho-HER2, NR2F1 and Wnt4 were performed as mentioned above. For apoptosis detection, cells were stained with Apotracker green (Cat. no. 427402, Biolegend) following manufacturer's instructions. RNA sequencing was performed as detailed above.

### 27. HER2-DC1 Intratumoral Delivery in HER2+ Breast Cancer Patients

Patients with HER2+, stage I-III invasive carcinoma of the breast were enrolled following informed consent on a Phase II neoadjuvant study at Moffitt Cancer Center (NCT05325632). Dendritic cells were generated as previously described and injected intratumorally via ultrasound guidance once weekly for 6 weeks. During this period, patients also received 2 infusions of trastuzumab and pertuzumab. Patients then went on to receive 12 weeks of paclitaxel and additional trastuzumab and pertuzumab according to standard of care guidelines, followed by surgery. Tumor biopsies were done at baseline and prior to chemotherapy. The trial was approved by the Moffitt Cancer Center institutional review board (protocol ID: MCC20915) and conducted in accordance with the Declaration of Helsinki and Good Clinical Practice Guidelines.

### 28. Multiplex Immunofluorescence and Quantitative Image Analysis

FFPE breast cancer biopsies were immunostained on the BOND RX autostainer (Leica Biosystems, Vista, CA) using the PerkinElmer OPAL™ 7-Color Automation IHC kit (Waltham, MA). The following antibodies were used for multiplex immunofluorescence (mIF) staining: anti-HER2 (Cat. No: 42905, Cell signaling technology), anti-PR (Cat. No: 8757S, Cell signaling technology), anti-Wnt4 (Cat. No: MAB4751, R&D Systems), anti-RANKL (Cat. No: ab45039, Abcam), anti-phospho-p38 (Cat. No: 4631S, Cell signaling technology), and anti-NR2F1 (Cat. No: ab224272, Abcam). Cancer cells were stained with anti-pan cytokeratin (Cat. No: M3515, Agilent Dako, Santa Clara, CA) and nuclei were counterstained with DAPI. Tumor tissue sections were heated for 2 hours at 65° C. and transferred to the BOND RX (Leica Biosystems). Next, deparaffinization and antigen retrieval was performed using the automated OPAL IHC procedure (PerkinElmer). All immunostained slides were then scanned and imaged using the PerkinElmer Vectra®3 Automated Quantitative Pathology Imaging System. Autofluorescence slides were included for a negative control. The multi-layer TIFF images were accessed and exported using the InForm software (PerkinElmer), loaded into HALO (Indica Labs, New Mexico) and quantitative image analysis was performed. Published cytoplasmic or nuclear staining patterns were utilized to determine the positivity thresholds for each marker.

### 29. Statistical Analysis

All data are presented as the mean±SEM. Statistical analysis was done using GraphPad Prism 8 software and Microsoft Excel. The unpaired or paired two-tailed Student's t test or one way ANOVA with Tukey's multiple comparisons test was used to calculate the p values. A p value less than 0.05 was considered statistically significant.

SEQUENCES

SEQ ID NO: 1 HER3 ECD P12 (aa 57-71)
EVVMGNLEIVLTGHN
SEQ ID NO: 2 HER3 ECD P81 (aa 401-415)
SWPPHMHNFSVFSNL
SEQ ID NO: 3 HER3 ECD P84 (aa 416-430)
TTIGGRSLYNRGFSL
SEQ ID NO: 4 HER3 ECD P91 (aa 450-464)
SAGRIYISANRQLCY
SEQ ID NO: 5 HER3 ICD P38 (aa 850-864)
VADFGVADLLPPDDK
SEQ ID NO: 6 HER3 ICD P41 (aa 865-879)
QLLYSEAKTPIKWMA
SEQ ID NO: 7 HER3 ICD P52 (aa 920-934)
VPDLLEKGERLAQPQ
SEQ ID NO: 8 HER3 ECD P12 (aa 56-70)
CEVVMGNLEIVLTGH
SEQ ID NO: 9 HER3 ECD P91 (aa 450-465)
SAGRIYISANRQLCYH
SEQ ID NO: 10 HER3 ICD P86 (aa 1090-1114)
GCLASESSEGHVTGS
SEQ ID NO: 11 HER3 ICD P89 (aa 1115-1129)
EAELQEKVSMCRSRS
SEQ ID NO: 12 HER2 MHC Class II P42-56
HLDMLRHLYQGCQVV
SEQ ID NO: 13 HER2 MHC Class II P98-114
RLRIVRGTQLFEDNYAL
SEQ ID NO: 14 HER2 MHC Class II P328-345
TQRCEKCSKPCARVCYGL
SEQ ID NO: 15 HER2 MHC Class II P776-790
GVGSPYVSRLLGICL
SEQ ID NO: 16 HER2 MHC Class II P927-941
PAREIPDLLEKGERL
SEQ ID NO: 17 HER2 MHC Class II P1166-1180
TLERPKTLSPGKNGV
SEQ ID NO: 18 HLA A2 restricted HER2 peptide (cross reacts with HER3) P369-377
KIFGSLAFL
SEQ ID NO: 19 HLA A2 restricted HER2 peptide (cross reacts with HER3) P698-697
RLLQETELV
SEQ ID NO: 20 MHC class II recognizing multi-epitope peptides p5
ELAAWCRWGFLLALLPPGIAG
SEQ ID NO: 21 MHC class II recognizing multi-epitope peptides p435
IRGRILHDGAYSLTLQGLGIH
SEQ ID NO: 22 MHC class II recognizing multi-epitope peptides p1209
SPPHPSPAFSPAFDNLYYWDQ
SEQ ID NO: 23 HER3 ECD P12
CEVVMGNLEIVLTGH
SEQ ID NO: 24 HER3 ECD P91
AGRIYISANRQLCYH

### REFERENCES

Anders, S., Pyl, P. T., and Huber, W. (2015). HTSeq—a Python framework to work with high-throughput sequencing data. Bioinformatics 31, 166-169. 10.1093/bioinformatics/btu638.
Artzy-Randrup, Y., Epstein, T., Brown, J. S., Costa, R. L. B., Czerniecki, B. J., and Gatenby, R. A. (2021). Novel evolutionary dynamics of small populations in breast cancer adjuvant and neoadjuvant therapy. NPJ Breast Cancer 7, 26. 10.1038/s41523-021-00230-y.
Axelrod, M. L., Cook, R. S., Johnson, D. B., and Balko, J. M. (2019). Biological Consequences of MHC-II Expression by Tumor Cells in Cancer. Clin Cancer Res 25, 2392-2402. 10.1158/1078-0432.CCR-18-3200.
Basu, A., Albert, G. K., Awshah, S., Datta, J., Kodumudi, K. N., Gallen, C., Beyer, A., Smalley, K. S. M., Rodriguez, P. C., Duckett, D. R., et al. (2022). Identification of Immunogenic MHC Class II Human HER3 Peptides that Mediate Anti-HER3 CD4(+) Th1 Responses and Potential Use as a Cancer Vaccine. Cancer Immunol Res 10, 108-125. 10.1158/2326-6066.CIR-21-0454.
Basu, A., Ramamoorthi, G., Albert, G., Gallen, C., Beyer, A., Snyder, C., Koski, G., Disis, M. L., Czerniecki, B. J., and Kodumudi, K. (2021). Differentiation and Regulation of T(H) Cells: A Balancing Act for Cancer Immunotherapy. Front Immunol 12, 669474. 10.3389/fimmu.2021.669474.
Ben-Porath, I., Thomson, M. W., Carey, V. J., Ge, R., Bell, G. W., Regev, A., and Weinberg, R. A. (2008). An embryonic stem cell-like gene expression signature in poorly differentiated aggressive human tumors. Nat Genet 40, 499-507. 10.1038/ng.127.
Blasco, M. T., Espuny, I., and Gomis, R. R. (2022). Ecology and evolution of dormant metastasis. Trends Cancer 8, 570-582. 10.1016/j.trecan.2022.03.002.
Borgen, E., Rypdal, M. C., Sosa, M. S., Renolen, A., Schlichting, E., Lonning, P. E., Synnestvedt, M., Aguirre-Ghiso, J. A., and Naume, B. (2018). NR2F1 stratifies dormant disseminated tumor cells in breast cancer patients. Breast Cancer Res 20, 120. 10.1186/s13058-018-1049-0.
Brastianos, P. K., Carter, S. L., Santagata, S., Cahill, D. P., Taylor-Weiner, A., Jones, R. T., Van Allen, E. M., Lawrence, M. S., Horowitz, P. M., Cibulskis, K., et al. (2015). Genomic Characterization of Brain Metastases Reveals Branched Evolution and Potential Therapeutic Targets. Cancer Discov 5, 1164-1177. 10.1158/2159-8290.CD-15-0369.
Braun, S., Vogl, F. D., Naume, B., Janni, W., Osborne, M. P., Coombes, R. C., Schlimok, G., Diel, I. J., Gerber, B., Gebauer, G., et al. (2005). A pooled analysis of bone marrow micrometastasis in breast cancer. N Engl J Med 353, 793-802. 10.1056/NEJMoa050434.
Celia-Terrassa, T., Liu, D. D., Choudhury, A., Hang, X., Wei, Y., Zamalloa, J., Alfaro-Aco, R., Chakrabarti, R., Jiang, Y. Z., Koh, B. I., et al. (2017). Normal and cancerous mammary stem cells evade interferon-induced constraint through the miR-199a-LCOR axis. Nat Cell Biol 19, 711-723. 10.1038/ncb3533.
Demel, U. M., Boger, M., Yousefian, S., Grunert, C., Zhang, L., Hotz, P. W., Gottschlich, A., Kose, H., Isaakidis, K., Vonficht, D., et al. (2022). Activated SUMOylation restricts MHC class 1 antigen presentation to confer immune evasion in cancer. J Clin Invest 132. 10.1172/JCI152383.
Di Pilato, M., Kfuri-Rubens, R., Pruessmann, J. N., Ozga, A. J., Messemaker, M., Cadilha, B. L., Sivakumar, R., Cianciaruso, C., Warner, R. D., Marangoni, F., et al. (2021). CXCR6 positions cytotoxic T cells to receive critical survival signals in the tumor microenvironment. Cell 184, 4512-4530 e4522. 10.1016/j.cell.2021.07.015.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21. 10.1093/bioinformatics/bts635.
Dockry, E., O'Leary, S., Gleeson, L. E., Lyons, J., Keane, J., Gray, S. G., and Doherty, D. G. (2018). Epigenetic induction of CD1d expression primes lung cancer cells for killing by invariant natural killer T cells. Oncoimmunology 7, e1428156. 10.1080/2162402×.2018.1428156.
Ehmsen, S., Pedersen, M. H., Wang, G., Terp, M. G., Arslanagic, A., Hood, B. L., Conrads, T. P., Leth-Larsen, R., and Ditzel, H. J. (2019). Increased Cholesterol Biosynthesis Is a Key Characteristic of Breast Cancer Stem Cells Influencing Patient Outcome. Cell Rep 27, 3927-3938 e3926. 10.1016/j.celrep.2019.05.104.
Eyre, R., Alferez, D. G., Santiago-Gomez, A., Spence, K., McConnell, J. C., Hart, C., Simoes, B. M., Lefley, D., Tulotta, C., Storer, J., et al. (2019). Microenvironmental IL1beta promotes breast cancer metastatic colonisation in the bone via activation of Wnt signalling. Nat Commun 10, 5016. 10.1038/s41467-019-12807-0.
Ferris, S. T., Durai, V., Wu, R., Theisen, D. J., Ward, J. P., Bern, M. D., Davidson, J. T. t., Bagadia, P., Liu, T., Briseno, C. G., et al. (2020). cDC1 prime and are licensed by CD4(+) T cells to induce anti-tumour immunity. Nature 584, 624-629. 10.1038/s41586-020-2611-3.
Fridman, R., Benton, G., Aranoutova, I., Kleinman, H. K., and Bonfil, R. D. (2012). Increased initiation and growth of tumor cell lines, cancer stem cells and biopsy material in mice using basement membrane matrix protein (Cultrex or Matrigel) co-injection. Nat Protoc 7, 1138-1144. 10.1038/nprot.2012.053.
Gatenby, R. A., and Brown, J. S. (2020). Integrating evolutionary dynamics into cancer therapy. Nat Rev Clin Oncol 17, 675-686. 10.1038/s41571-020-0411-1.
Harper, K. L., Sosa, M. S., Entenberg, D., Hosseini, H., Cheung, J. F., Nobre, R., Avivar-Valderas, A., Nagi, C., Girnius, N., Davis, R. J., et al. (2016). Mechanism of early dissemination and metastasis in Her2(+) mammary cancer. Nature 540, 588-592. 10.1038/nature20609.
Hartkopf, A. D., Brucker, S. Y., Taran, F. A., Harbeck, N., von Au, A., Naume, B., Pierga, J. Y., Hoffmann, O., Beckmann, M. W., Ryden, L., et al. (2021). Disseminated tumour cells from the bone marrow of early breast cancer patients: Results from an international pooled analysis. Eur J Cancer 154, 128-137. 10.1016/j.ejca.2021.06.028.
Hartkopf, A. D., Wallwiener, M., Fehm, T. N., Hahn, M., Walter, C. B., Gruber, I., Brucker, S. Y., and Taran, F. A. (2015). Disseminated tumor cells from the bone marrow of patients with nonmetastatic primary breast cancer are predictive of locoregional relapse. Ann Oncol 26, 1155-1160. 10.1093/annonc/mdv148.
Hong, S., Zhang, Z., Liu, H., Tian, M., Zhu, X., Zhang, Z., Wang, W., Zhou, X., Zhang, F., Ge, Q., et al. (2018). B Cells Are the Dominant Antigen-Presenting Cells that Activate Naive CD4(+) T Cells upon Immunization with a Virus-Derived Nanoparticle Antigen. Immunity 49, 695-708 e694. 10.1016/j.immuni.2018.08.012.
Hosseini, H., Obradovic, M. M. S., Hoffmann, M., Harper, K. L., Sosa, M. S., Werner-Klein, M., Nanduri, L. K., Werno, C., Ehrl, C., Maneck, M., et al. (2016). Early dissemination seeds metastasis in breast cancer. Nature 540, 552-558. 10.1038/nature20785.
Huang, B., Song, B. L., and Xu, C. (2020). Cholesterol metabolism in cancer: mechanisms and therapeutic opportunities. Nat Metab 2, 132-141. 10.1038/s42255-020-0174-0.
Huang, Y., Wang, H., Yue, X., and Li, X. (2023). Bone serves as a transfer station for secondary dissemination of breast cancer. Bone Res 11, 21. 10.1038/s41413-023-00260-1.
Husemann, Y., Geigl, J. B., Schubert, F., Musiani, P., Meyer, M., Burghart, E., Forni, G., Eils, R., Fehm, T., Riethmuller, G., and Klein, C. A. (2008). Systemic spread is an early step in breast cancer. Cancer Cell 13, 58-68. 10.1016/j.ccr.2007.12.003.
Jassal, B., Matthews, L., Viteri, G., Gong, C., Lorente, P., Fabregat, A. Sidiropoulos, K., Cook, J., Gillespie, M., Haw, R., et al. (2020). The reactome pathway knowledgebase. Nucleic Acids Res 48, D498-D503. 10.1093/nar/gk
Jerusalem, G., Park, Y. H., Yamashita, T., Hurvitz, S. A., Modi, S., Andre, F., Krop, I. E., Gonzalez Farre, X., You, B., Saura, C., et al. (2022). Trastuzumab Deruxtecan in HER2-Positive Metastatic Breast Cancer Patients with Brain Metastases: A DESTINY-BreastOl Subgroup Analysis. Cancer Discov 12, 2754-2762. 10.1158/2159-8290.CD-22-0837.
Jones, D. H., Nakashima, T., Sanchez, O. H., Kozieradzki, I., Komarova, S. V., Sarosi, I., Morony, S., Rubin, E., Sarao, R., Hojilla, C. V., et al. (2006). Regulation of cancer cell migration and bone metastasis by RANKL. Nature 440, 692-696. 10.1038/nature04524.
Kang, Y., and Pantel, K. (2013). Tumor cell dissemination: emerging biological insights from animal models and cancer patients. Cancer Cell 23, 573-581. 10.1016/j.ccr.2013.04.017.
Kodumudi, K. N., Ramamoorthi, G., Snyder, C., Basu, A., Jia, Y., Awshah, S., Beyer, A. P., Wiener, D., Lam, L., Zhang, H., et al. (2019). Sequential Anti-PD1 Therapy Following Dendritic Cell Vaccination Improves Survival in a HER2 Mammary Carcinoma Model and Identifies a Critical Role for CD4 T Cells in Mediating the Response. Front Immunol 10, 1939. 10.3389/fimmu.2019.01939.
Lameris, R., Shahine, A., Pellicci, D. G., Uldrich, A. P., Gras, S., Le Nours, J., Groen, R. W. J., Vree, J., Reddiex, S. J. J., Quinones-Parra, S. M., et al. (2020). A single-domain bispecific antibody targeting CDId and the NKT T-cell receptor induces a potent antitumor response. Nat Cancer 1, 1054-1065. 10.1038/s43018-020-00111-6.
Linde, N., Casanova-Acebes, M., Sosa, M. S., Mortha, A., Rahman, A., Farias, E., Harper, K., Tardio, E., Reyes Torres, I., Jones, J., et al. (2018). Macrophages orchestrate breast cancer early dissemination and metastasis. Nat Commun 9, 21. 10.1038/s41467-017-02481-5.
Liu, S., Galat, V., Galat, Y., Lee, Y. K. A., Wainwright, D., and Wu, J. (2021). NK cell-based cancer immunotherapy: from basic biology to clinical development. J Hematol Oncol 14, 7. 10.1186/s13045-020-01014-w.
Liu, S. S., Qi, J., Teng, Z. D., Tian, F. T., Lv, X. X., Li, K., Song, Y. J., Xie, W. D., Hu, Z. W., and Li, X. (2020). Resistomycin attenuates triple-negative breast cancer progression by inhibiting E3 ligase Pellino-1 and inducing SNAIL/SLUG degradation. Signal Transduct Target Ther 5, 133. 10.1038/s41392-020-00255-y.
Liu, Y., Gu, Y., Han, Y., Zhang, Q., Jiang, Z., Zhang, X., Huang, B., Xu, X., Zheng, J., and Cao, X. (2016). Tumor Exosomal RNAs Promote Lung Pre-metastatic Niche Formation by Activating Alveolar Epithelial TLR3 to Recruit Neutrophils. Cancer Cell 30, 243-256. 10.1016/j.ccell.2016.06.021.
Liu, Y., Zhang, P., Wu, Q., Fang, H., Wang, Y., Xiao, Y., Cong, M., Wang, T., He, Y., Ma, C., et al. (2021). Long non-coding RNA NR2F1-AS1 induces breast cancer lung metastatic dormancy by regulating NR2F1 and DeltaNp63. Nat Commun 12, 5232. 10.1038/s41467-021-25552-0.
Love, M. I., Huber, W., and Anders, S. (2014). Moderated estimation of fold change and dispersion for RNA-seq data with DESeq2. Genome Biol 15, 550. 10.1186/s13059-014-0550-8.
Lowenfeld, L., Mick, R., Datta, J., Xu, S., Fitzpatrick, E., Fisher, C. S., Fox, K. R., DeMichele, A., Zhang, P. J., Weinstein, S. P., et al. (2017). Dendritic Cell Vaccination Enhances Immune Responses and Induces Regression of HER2(pos) DCIS Independent of Route: Results of Randomized Selection Design Trial. Clin Cancer Res 23, 2961-2971. 10.1158/1078-0432.CCR-16-1924.
Magbanua, M. J. M., Rugo, H. S., Hauranieh, L., Roy, R., Scott, J. H., Lee, J. C., Hsiao, F., Sosa, E. V., Van′t Veer, L., Esserman, L. J., and Park, J. W. (2018). Genomic and expression profiling reveal molecular heterogeneity of disseminated tumor cells in bone marrow of early breast cancer. NPJ Breast Cancer 4, 31. 10.1038/s41523-018-0083-5.
Malta, T. M., Sokolov, A., Gentles, A. J., Burzykowski, T., Poisson, L., Weinstein, J. N., Kaminska, B., Huelsken, J., Omberg, L., Gevaert, O., et al. (2018). Machine Learning Identifies Sternness Features Associated with Oncogenic Dedifferentiation. Cell 173, 338-354 e315. 10.1016/j.cell.2018.03.034.
Martin, M. (2011). Cutadapt removes adapter sequences from high-throughput sequencing reads. EMBnet. journal 17, 10-12.
Naume, B., Synnestvedt, M., Falk, R. S., Wiedswang, G., Weyde, K., Risberg, T., Kersten, C., Mjaaland, I., Vindi, L., Sommer, H. H., et al. (2014). Clinical outcome with correlation to disseminated tumor cell (DTC) status after DTC-guided secondary adjuvant treatment with docetaxel in early breast cancer. J Clin Oncol 32, 3848-3857. 10.1200/JCO.2014.56.9327.
Oechsle, C. M., Showalter, L. E., Novak, C. M., Czerniecki, B. J., and Koski, G. K. (2020). Statin Drugs Plus Th1 Cytokines Potentiate Apoptosis and Ras Delocalization in Human Breast Cancer Lines and Combine with Dendritic Cell-Based Immunotherapy to Suppress Tumor Growth in a Mouse Model of HER-2(pos) Disease. Vaccines (Basel) 8. 10.3390/vaccines8010072.
Ouzounova, M., Lee, E., Piranlioglu, R., El Andaloussi, A., Kolhe, R., Demirci, M. F., Marasco, D., Asm, I., Chadli, A., Hassan, K. A., et al. (2017). Monocytic and granulocytic myeloid derived suppressor cells differentially regulate spatiotemporal tumour plasticity during metastatic cascade. Nat Commun 8, 14979. 10.1038/ncomms14979.
Ozga, A. J., Chow, M. T., and Luster, A. D. (2021). Chemokines and the immune response to cancer. Immunity 54, 859-874. 10.1016/j.immuni.2021.01.012.
Piranlioglu, R., Lee, E., Ouzounova, M., Bollag, R. J., Vinyard, A. H., Arbab, A. S., Marasco, D., Guzel, M., Cowell, J. K., Thangaraju, M., et al. (2019). Primary tumor-induced immunity eradicates disseminated tumor cells in syngeneic mouse model. Nat Commun 10, 1430. 10.1038/s41467-019-09015-1.
Rajapakse, V. N., Luna, A., Yamade, M., Loman, L., Varma, S., Sunshine, M., Iorio, F., Sousa, F. G., Elloumi, F., Aladjem, M. I., et al. (2018). CellMinerCDB for Integrative Cross-Database Genomics and Pharmacogenomics Analyses of Cancer Cell Lines. iScience 10, 247-264. 10.1016/j.isci.2018.11.029.
Ramamoorthi, G., Kodumudi, K., Gallen, C., Zachariah, N. N., Basu, A., Albert, G., Beyer, A., Snyder, C., Wiener, D., Costa, R. L. B., and Czerniecki, B. J. (2022). Disseminated cancer cells in breast cancer: Mechanism of dissemination and dormancy and emerging insights on therapeutic opportunities. Semin Cancer Biol 78, 78-89. 10.1016/j.semcancer.2021.02.004.
Ramamoorthi, G., Kodumudi, K., Snyder, C., Grover, P., Zhang, H., Greene, M. I., Basu, A., Gallen, C., Wiener, D., Costa, R. L. B., et al. (2022). Intratumoral delivery of dendritic cells plus anti-HER2 therapy triggers both robust systemic antitumor immunity and complete regression in HER2 mammary carcinoma. J Immunother Cancer 10. 10.1136/jitc-2022-004841.
Rhim, A. D., Mirek, E. T., Aiello, N. M., Maitra, A., Bailey, J. M., McAllister, F., Reichert, M., Beatty, G. L., Rustgi, A. K., Vonderheide, R. H., et al. (2012). EMT and dissemination precede pancreatic tumor formation. Cell 148, 349-361. 10.1016/j.cell.2011.11.025.
Rocken, M. (2010). Early tumor dissemination, but late metastasis: insights into tumor dormancy. J Clin Invest 120, 1800-1803. 10.1172/JCI43424.
Rodriguez-Tirado, C., Kale, N., Carlini, M. J., Shrivastava, N., Rodrigues, A. A., Khalil, B. D., Bravo-Cordero, J. J., Hong, Y., Alexander, M., Ji, J., et al. (2022). NR2F1 Is a Barrier to Dissemination of Early-Stage Breast Cancer Cells. Cancer Res 82, 2313-2326. 10.1158/0008-5472.CAN-21-4145.
Schmitt, C. A., Wang, B., and Demaria, M. (2022). Senescence and cancer—role and therapeutic opportunities. Nat Rev Clin Oncol 19, 619-636. 10.1038/s41571-022-00668-4.
Siddiqui, I., Erreni, M., van Brakel, M., Debets, R., and Allavena, P. (2016). Enhanced recruitment of genetically modified CX3CR1-positive human T cells into Fractalkine/CX3CL1 expressing tumors: importance of the chemokine gradient. J Immunother Cancer 4, 21. 10.1186/s40425-016-0125-1.
Spanoudakis, E., Hu, M., Naresh, K., Terpos, E., Melo, V., Reid, A., Kotsianidis, I., Abdalla, S., Rahemtulla, A., and Karadimitris, A. (2009). Regulation of multiple myeloma survival and progression by CD1d. Blood 113, 2498-2507. 10.1182/blood-2008-06-161281.
Spiegel, A., Brooks, M. W., Houshyar, S., Reinhardt, F., Ardolino, M., Fessler, E., Chen, M. B., Krall, J. A., DeCock, J., Zervantonakis, I. K., et al. (2016). Neutrophils Suppress Intraluminal NK Cell-Mediated Tumor Cell Clearance and Enhance Extravasation of Disseminated Carcinoma Cells. Cancer Discov 6, 630-649. 10.1158/2159-8290.CD-15-1157.
Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. Proc Natl Acad Sci USA 102, 15545-15550. 10.1073/pnas.0506580102.
Suresh, S., Rabbie, R., Garg, M., Lumaquin, D., Huang, T. H., Montal, E., Ma, Y., Cruz, N. M., Tang, X., Nsengimana, J., et al. (2023). Identifying the Transcriptional Drivers of Metastasis Embedded within Localized Melanoma. Cancer Discov 13, 194-215. 10.1158/2159-8290.CD-22-0427.
Tran, H. D., Luitel, K., Kim, M., Zhang, K., Longmore, G. D., and Tran, D. D. (2014). Transient SNAIL1 expression is necessary for metastatic competence in breast cancer. Cancer Res 74, 6330-6340. 10.1158/0008-5472.CAN-14-0923.
Trapnell, C., Pachter, L., and Salzberg, S. L. (2009). TopHat: discovering splice junctions with RNA-Seq. Bioinformatics 25, 1105-1111. 10.1093/bioinformatics/btp120.
Vargas, G., Bouchet, M., Bouazza, L., Reboul, P., Boyault, C., Gervais, M., Kan, C., Benetollo, C., Brevet, M., Croset, M., et al. (2019). ERRalpha promotes breast cancer cell dissemination to bone by increasing RANK expression in primary breast tumors. Oncogene 38, 950-964. 10.1038/s41388-018-0579-3.
Volmer, L., Koch, A., Matovina, S., Dannehl, D., Weiss, M., Welker, G., Hahn, M., Engler, T., Wallwiener, M., Walter, C. B., et al. (2022). Neoadjuvant Chemotherapy of Patients with Early Breast Cancer Is Associated with Increased Detection of Disseminated Tumor Cells in the Bone Marrow. Cancers (Basel) 14. 10.3390/cancers14030635.
Wendel, M., Galani, I. E., Suri-Payer, E., and Cerwenka, A. (2008). Natural killer cell accumulation in tumors is dependent on IFN-gamma and CXCR3 ligands. Cancer Res 68, 8437-8445. 10.1158/0008-5472.CAN-08-1440.
Wimberger, P., Blohmer, J. U., Krabisch, P., Link, T., Just, M., Sinn, B. V., Simon, E., Solbach, C., Fehm, T., Denkert, C., et al. (2023). The effect of denosumab on disseminated tumor cells (DTCs) of breast cancer patients with neoadjuvant treatment: a GeparX translational substudy. Breast Cancer Res 25, 32. 10.1186/s13058-023-01619-2.
Yuseff, M. I., Pierobon, P., Reversat, A., and Lennon-Dumenil, A. M. (2013). How B cells capture, process and present antigens: a crucial role for cell polarity. Nat Rev Immunol 13, 475-486. 10.1038/nri3469.

1. A method of reducing cancer cell dissemination and/or metastasis in a subject with a cancer comprising administering to the subject tumor antigen-pulsed Type 1 conventional dendritic cells (cDC1).

2. The method of claim 1, wherein the cancer is a breast cancer.

3. The method of claim 1, wherein the cancer is a melanoma.

4. The method of claim 1, wherein the method further reduces a second cancer at a site distant from a tumor microenvironment of the cancer being targeted for treatment.

5. The method of claim 1, wherein the tumor antigen-pulsed Type 1 cDC1 are administered intratumorally.

6. The method of claim 1, wherein the tumor antigen comprises a CD4 T cell epitope from an oncodriver.

7. The method of claim 6, wherein the oncodriver comprises human epidermal growth factor receptor (HER) 1 (HER1), HER2, HER3, epidermal growth factor receptor (EGFR), c-Mesenchymal to Epithelial Transition (c-MET), B-Rapidly Accelerated Fibrosarcoma (BRAF), KIT, Androgen Receptor (AR), Estrogen Receptor (ER), Kirsten rat sarcoma (KRAS), TP53, or APC.

8. The method of claim 6, wherein the oncodriver comprises class II peptides which do not require an HLA match.

9. A method of reducing cancer cell dissemination and/or metastasis in a subject with a cancer comprising:
(i) obtaining CD4Th1 T cells;
(ii) contacting the CD4 T cells with tumor antigen pulsed Type 1 conventional dendritic cells thereby creating DC1 primed CD4 T cells; and
(iii) administering to the subject the DC1 primed CD4 T cells.

10. The method of claim 9, wherein the cancer is a breast cancer.

11. The method of claim 9, wherein the cancer is a melanoma.

12. The method of claim 9, wherein the method further reduces a second cancer at a site distant from a tumor microenvironment of the cancer being targeted for treatment.

13. The method of claim 9, wherein the DC1 primed CD4 T cells are administered intratumorally.

14. The method of claim 9, wherein the tumor antigen comprises a CD4 T cell epitope from an oncodriver.

15. The method of claim 14, wherein the oncodriver comprises HER1, HER2, HER3, EGFR, c-MET c, BRAF, KIT, AR, ER, KRAS, TP53, or APC

16. The method of claim 14, wherein the oncodriver comprises class II peptides which do not require an HLA match.

17. The method of claim 14, wherein the tumor antigen comprising the CD4 T cell epitope from an oncodriver induces the increased expression of Interferon-γ (IFN-γ).

18. The method of claim 1, further comprising obtaining a tissue sample from the subject, and performing Prelude DCIS identification assay to detect the presence of disseminated cancer cells (DCC) in the subject.

19. The method of claim 18, wherein the tissue sample comprises blood, bone marrow, or tissue biopsy.

20. A method of treating cancer metastasis in a subject, wherein the method comprises;
(i) obtaining a tissue sample from the subject;
(ii) performing imaging or a proteomic assay of the tissue sample to detect the presence of DCC in the subject; and
(iii) if the presence of DCC in the subject is seen, then treating the subject with a a treatment selected from hormone therapy, immunotherapy, targeted therapy, chemotherapy, or combinations thereof.

21. The method of claim 20, wherein the tissue sample comprises blood, bone marrow, or tissue biopsy.
